(19) United States
(12) Patent Application Publication
(10) Pub. No.: US 2010/0035862 A1
(43) Pub. Date: Feb. 11, 2010

(54) NOVEL AZA-CYCLIC INDOLE-2-CARBOXAMIDES AND METHODS OF USE THEREOF

(75) Inventors: Murali Gopalakrishnan, Libertyville, IL (US); Jianguo Ji, Libertyville, IL (US); Chih-Hung Lee, Vernon Hills, IL (US); Tao Li, Grayslake, IL (US); Kevin B. Sippy, Antioch, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, Abbott Park, IL (US)

(21) Appl. No.: 12/490,569

(22) Filed: Jun. 24, 2009

Related U.S. Application Data
(60) Provisional application No. 61/075,593, filed on Jun. 25, 2008.

(51) Int. Cl. A61K 31/55; C07D 403/02; A61K 31/403; C07D 401/02; A61K 31/454

(52) U.S. Cl. 514/217.08; 548/465; 514/414; 546/201; 514/323; 540/602

(57) ABSTRACT

The invention relates to aza-cyclic-indole-2-carboxamide derivatives, compositions comprising such compounds, and methods of preventing or treating conditions and disorders using such compounds and compositions.

This application claims priority to U.S. patent application Ser. No. 61/075,593 filed on Jun. 25, 2008 and is incorporated herein by reference.

### TECHNICAL FIELD

The invention relates to novel aza-cyclic indole-2-carboxamide derivatives, compositions comprising such compounds, and methods of preventing or treating conditions and disorders using such compounds and compositions.

### DESCRIPTION OF RELATED TECHNOLOGY

The endogenous cholinergic neurotransmitter, acetylcholine (ACh), exerts its biological effect via two types of cholinergic receptors, the muscarinic acetylcholine receptors (mAChR) and the nicotinic acetylcholine receptors (nAChR). nAChRs are pentameric assemblies of subunits surrounding a central pore that gates the flux of Na⁺, K⁺ and Ca²⁺ ions. At least 16 subunit proteins, i.e. α2-α10, β1-β10, γ, δ and ε, have been identified in neuronal tissues. These subunits provide for a great variety of homomeric and heteromeric combinations that account for the diverse receptor subtypes. For example, functional neuronal nAChR or neuronal nicotinic receptor (NNR) assemblies can be homomeric, comprising α7, α8 or α9 subunits, or heteromeric, usually with at least one subunit from the α group (α2, α3, α4 and α6) and the remainder from the β group (β2 and β4). In the central nervous system, α4β2-containing NNR and α7-containing NNR subtypes are the most widespread and mediate synaptic and, possibly, paracrine functions. These NNRs are expressed at high levels in areas involved with learning and memory, and play key roles in modulating neurotransmission in these regions. Reduced cholinergic activity and dysregulation of NNRs have been correlated with disease states involving cognitive deficits, progressive dementia, and epilepsy. Accordingly, these NNRs are implicated in a range of physiological and patho-physiological functions related to cognitive function, learning and memory, reward, motor control, arousal and analgesia (reviewed in Gopalakrishnan, M. et al., Ion channels—Ligand-gated. Comprehensive Medicinal Chemistry II, Edited by Triggle D. J., et al., Major Reference Works, Elsevier. Unit 2.22, pp 877-918, 2006).

Discovery of the important roles played by NNRs in several CNS disorders has called attention to these membrane proteins and to ligands, or compounds, that are able to modulate, i.e. modify, the function of such membrane proteins. The prototypical NNR agonist, nicotine, has itself been shown to improve attention and cognitive performance, reduce anxiety, normalize sensory gating, and effect neuroprotection. However, nicotine is not sufficiently selective among NNRs and its utility is limited by side effects including seizures, irregular heartbeat, hypertension, and gastrointestinal effects. Accordingly, identification of compounds, agonists or allostertc modulators, that target distinct subtypes to retain the beneficial effects, while eliminating or decreasing adverse effects, continues to be an active area of research.

NNRs, especially α4β2 NNRs, have been targeted for pain, cognitive disorders and various central nervous system diseases. Gene knockout, antisense and pharmacological studies have shown that α4 and β2 NNRs are responsible for mediating nicotinic analgesia at supraspinal responses and spinal sites (Decker, M. W., et al., Curr. Top. Med. Chem., 4: 369-384, 2004). Ligands targeting α4β2 NNRs have shown improvement in cognitive and attentive function in preclinical models and, more recently, in human disease states such as attention deficit hyperactivity disorder (ADHD) (Wilens, T. E., et al., Biol. Psychiatry, 59: 1065, 2006) and age-associated memory impairment (Dunbar, G. C., et al., Psychopharmacol., 21: 171, 2007). A key goal in the discovery of novel NNR compounds is to avoid ganglioinic α3* NNRs, as the dose-limiting emetic liability of nonselective compounds may be attributed to activation of α3 containing NNRs. α3* NNRs in the dorsal motor nucleus of the vagus and in nucleus tractus solitarius have been implicated in gastric and blood pressure responses to nicotine injected locally (Ferreira, M., et al., J. Pharmacol. Exp. Ther. 294:230-238, 2000).

Compounds with varying degrees of selectivity for α4β2 NNRs over other nicotinic subtypes (α3, α7, α1-containing) have been discovered over the years for the treatment of pain and a range of psychiatric and neurological disorders especially involving cognitive deficits in attention, alertness and memory. These may include those conditions that may benefit from selective enhancement of cholinergic transmission such as attention deficit, psychotic disorders, selected pain syndromes smoking cessation and those thought to involve reduced cholinergic function such as neurodegenerative disorders, central inflammatory or autoimmune disorders, brain trauma and cerebrovascular disease. Modulation of α4β2 NNRs is expected to be beneficial in a number of diseases including Alzheimer's disease, mild cognitive impairment and related syndromes, Lewy body dementia, vascular dementia, attention deficit/attention deficit-hyperactivity disorder, schizophrenia, bipolar and mood disorders, schizoaffective disorders, Tourette's syndrome, brain trauma, vascular dementia, Parkinson's disease, Huntington's disease and conditions of substance abuse including alcohol abuse and smoking cessation. Selected pain syndromes includes chronic pain that can be nociceptive, neuropathic, or both and originating from cancer, injury, surgery, or chronic conditions such as arthritis or nerve injury/disease. Neuropathic pain can be peripheral (painful peripheral mononeuropathy and polyneuropathy) or central (post stroke, following spinal cord injury) and can originate from nerve injury following a wide-array of conditions/events such as direct trauma to nerves, inflammation/neuritis/nerve compression, metabolic diseases (diabetes), infections (herpes zoster, HIV), tumors, toxins (chemotherapy), and primary neurological diseases.

Treatment with NNR agonists, which act at the same site, as the endogenous transmitter ACh, may be problematic because ACh not only activates, but also inhibits receptor activity through processes that include desensitization. Further, prolonged receptor activation may cause long-lasting inactivation. Thus, uncertainty exists whether chronic treatment with agonists in humans-might provide suboptimal benefit due to sustained receptor activation and desensitization of the NNRs. An alternate approach to target α4β2 NNR function is by enhancing effects of the endogenous neurotransmitter acetylcholine via positive allostertc modulation. This approach provides an opportunity to (i) reinforce the endogenous cholinergic neurotransmission without directly activating the receptor like classical agonists, (ii) prevent receptor desensitization (iii) possibly resensitize inactivated receptors. Thus, the spatial and temporal characteristics of endogenous α4β2 receptor activation are preserved unlike agonists that will tonically activate all receptors, leading to a non-physiological pattern of receptor activation.

In light of the evidence supporting the various therapeutic uses of NNRs, it would be, beneficial to discover novel allostertc modulators that could provide therapeutic benefits.

### SUMMARY OF THE INVENTION

The invention relates to novel aza-cyclic indole-2-carboxamide compounds, compositions comprising such compounds, and method of using such compounds and compositions.

In one aspect, the invention is compounds having the formula (I)

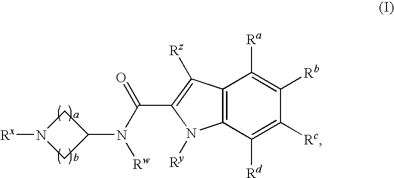

wherein a and b are independently 1, 2, 3, or 4; R^{a}, R^{b}, R^{c} and R^{d} are independently hydrogen, alkyl, aryl, cyano, halogen, haloalkyl, heteroaryl, NR¹R², nitro, OR³, SR¹, or SO₂R¹; or R^{a}, R^{b}, and the carbon atoms to which they are attached taken together form a monocyclic aryl or monocyclic heteroaryl; R¹ and R² are independently hydrogen, alkyl, arylalkyl, or cycloalkyl; R³ is hydrogen, alkyl, arylalkyl, cycloalkyl, or haloalkyl; R^{w} is hydrogen or alkyl; R^{x} and R^{y} are independently hydrogen, alkyl, or cycloalkyl; and R^{z} is hydrogen, alkyl, aryl, or heteroaryl.

Another aspect of the invention relates to pharmaceutical compositions comprising compounds of formula (I). Such compositions can be administered typically as part of a therapeutic regimen for treatment, or prevention of conditions and disorders related to NNR activity.

Yet another aspect of the invention relates to a method of modulating α4β2 NNR activity. The method is useful for treating, preventing or both treating and preventing conditions and disorders related to α4β2 NNR activity, particularly in mammals. Such method is useful for treating, preventing or both treating and preventing conditions and disorders related to α4β2 NNR activity in mammals.

A further aspect of the invention relates to a method of selectively modulating NNR activity, for example α4β2 NNR positive allostertc modulator (PAM) activity, in combination with a nicotinic agonist or partial agonist to improve the tolerability of therapy using such nicotinic agonist or partial agonist.

Yet another aspect of the invention relates to a method for treating, preventing or both treating and preventing pain.

### DETAILED DESCRIPTION OF THE INVENTION

### Definition of Terms

As used throughout this specification and the appended claims, the following terms have the following meanings:

The term “acetyl” means a —C(O)CH₃ group.

The term “alkenyl” means a straight or branched chain hydrocarbon containing from 2 to 10 carbons and containing at least one carbon-carbon double bond formed by the removal of two hydrogens. Representative examples of alkenyl include, but are not limited to, ethenyl and various isomers of propenyl, butenyl, pentenyl, hexenyl, heptenyl, octenyl, nonenyl, and decenyl, such as 2-propenyl, 2-methyl-2-propenyl, 3-butenyl, 4-pentenyl, 5-hexenyl, 2-heptenyl, 2-methyl-1-heptenyl, and 3-decenyl.

The term “alkoxy” means an alkyl group appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxyalkoxy” means an alkoxy group appended to the parent molecular moiety through another alkoxy group, as defined herein. Representative examples of alkoxyalkoxy include, but are not limited to, tert-butoxymethoxy, 2-ethoxyethoxy, 2-methoxyethoxy, and methoxymethoxy.

The term “alkoxyalkoxyalkyl” means an alkoxyalkoxy group appended to the parent molecular moiety through an alkyl group, as defined herein. Representative examples of alkoxyalkoxyalkyl include, but are not limited to, tert-butoxymethoxymethyl, ethoxymethoxymethyl, (2-methoxyethoxy)methyl, and 2-(2-methoxyethoxy)ethyl.

The term “alkoxyalkyl” means an alkoxy group appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkoxycarbonyl” means an alkoxy group appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkoxycarbonylalkyl” means an alkoxycarbonyl group appended to the parent molecular moiety through an alkyl group, as defined herein. Representative examples of alkoxycarbonylalkyl include, but are not limited to, 3-methoxycarbonylpropyl, 4-ethoxycarbonylbutyl, and 2-tert-butoxycarbonylethyl.

The term “alkoxysulfonyl” means an alkoxy group appended to the parent molecular moiety through a sulfonyl group, as defined herein. Representative examples of alkoxysulfonyl include, but are not limited to, methoxysulfonyl, ethoxysulfonyl and propoxysulfonyl.

The term “alkyl” means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylamino” means an alkyl group appended to the parent molecular moiety through an amino group, as defined herein. Representative examples of alkylamino include, but are not limited to, methylamino, ethylamino, and sec-butylamino.

The term “alkylcarbonyl” means an alkyl group appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonylalkyl” means an alkylcarbonyl group appended to the parent molecular moiety through an alkyl group, as defined herein. Representative examples of alkylcarbonylalkyl include, but are not limited to, 2-oxopropyl, 3,3-dimethyl-2-oxopropyl, 3-oxobutyl, and 3-oxopentyl.

The term “alkylcarbonyloxy” means an alkylcarbonyl group appended to the parent molecular moiety through an oxygen atom. Representative examples of alkylcarbonyloxy include, but are not limited to, acetyloxy, ethylcarbonyloxy, and tert-butylcarbonyloxy.

The term “alkylcarbonyloxylalkyl” means an alkylcarbonyloxy group appended to the parent molecular moiety through an alkyl group.

The term “alkylene” means a divalent group derived from a straight or branched chain hydrocarbon of from 1 to 10 carbon atoms. Representative examples of alkylene include, but are not limited to, —CH₂—, —CH(CH₃)—, —C(CH₃)₂—, —CH₂CH₂—, —CH₂CH₂CH₂—, —CH₂CH₂CH₂CH₂—, and —CH₂CH(CH₃)CH₂—.

The term “alkylsulfinyl” means an alkyl group appended to the parent molecular moiety through a sulfinyl group, as defined herein. Representative examples of alkylsulfinyl include, but are not limited to, methylsulfinyl and ethylsulfinyl.

The term “alkylsulfinylalkyl” means an alkylsulfinyl group appended to the parent molecular moiety through an alkyl group, as defined herein. Representative examples of alkylsulfinylalkyl include, but are not limited to, methylsulfinylmethyl and ethylsulfinylmethyl.

The term “alkylsulfonyl” means an alkyl group appended to the parent molecular moiety through a sulfonyl group, as defined herein. Representative examples of alkylsulfonyl include, but are not limited to, methylsulfonyl and ethylsulfonyl.

The term “alkylsulfonylalkyl” means an alkylsulfonyl group appended to the parent molecular moiety through an alkyl group, as defined herein. Representative examples of alkylsulfonylalkyl include, but are not limited to, methylsulfonylmethyl and ethylsulfonylmethyl.

The term “alkylthio” means an alkyl group appended to the parent molecular moiety through a sulfur atom. Representative examples of alkylthio include, but are not limited, methylthio, ethylthio, tert-butylthio, and hexylthio.

The term “alkylthioalkyl” means an alkylthio group appended to the parent molecular moiety through an alkyl group, as defined herein. Representative examples of alkylthioalkyl include, but are not limited, methylthiomethyl and 2-(ethylthio)ethyl.

The term “alkynyl” means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “amino” means —NH₂, —NZ¹Z² or —NZ³Z⁴ group.

The term “aryl” means phenyl, a bicyclic aryl or a tricyclic aryl. The bicyclic aryl is naphthyl, a phenyl fused to a cycloalkyl, or a phenyl fused to a cycloalkenyl. Representative examples of the bicyclic aryl include, but are not limited to dihydroindenyl, indenyl, naphthyl, dihydronaphthalenyl, and tetrahydronaphthalenyl. The tricyclic aryl is anthracene or phenanthrene, or a bicyclic aryl fused to a cycloalkyl, or a bicyclic aryl fused to a cycloalkenyl, or a bicyclic aryl fused to a phenyl.

The aryl groups of this invention can be substituted with 0, 1, 2, 3, 4 or 5 substituents independently selected from alkenyl, alkoxy, alkoxyalkoxy, alkoxyalkoxyalkyl, alkoxyalkyl, alkoxycarbonyl, alkoxycarbonylalkyl, alkyl, alkylcarbonyl, alkylcarbonylalkyl, alkylcarbonyloxy, alkylcarbonyloxyalkyl, alkylsulfinyl, alkylsulfinylalkyl, alkylsulfonyl, alkylsulfonylalkyl, alkylthio, alkylthioalkyl, alkynyl, arylalkyl, arylalkoxy, aryloxy, carboxy, carboxyalkyl, cyano, cyanoalkyl, formyl, formylalkyl, halogen, haloalkyl, haloalkoxy, hydroxy, hydroxyalkyl, mercapto, nitro, —NZ¹Z², and (NZ³Z⁴)carbonyl.

The term “arylalkoxy” means an aryl group appended to the parent molecular moiety through an alkoxy group, as defined herein. Representative examples of arylalkoxy include, but are not limited to, 2-phenylethoxy, 3-naphth-2-ylpropoxy, and 5-phenylpentyloxy.

The term “arylalkyl” means an aryl group appended to the parent molecular-moiety through an alkyl group, as defined herein. Representative examples of arylalkyl include, but are not limited to, benzyl, 2-phenylethyl, 3-phenylpropyl, and 2-naphth-2-ylethyl.

The term “aryloxy” means an aryl group appended to the parent molecular moiety through an oxygen atom. Representative examples of aryloxy include, but are not limited to, phenoxy, naphthyloxy, 3-bromophenoxy, 4-chlorophenoxy, 4-methylphenoxy, and 3,5-dimethoxyphenoxy.

The term “aza-cycle” or “aza-cyclic” means a 3-, 4-, 5-, 6-, or 7-membered monocyclic heterocycle containing 1 nitrogen atom. Representative examples of aza-cyclic include, but are not limited to aziridinyl, azetidinyl, pyrrolidinyl, piperidinyl, and azapanyl.

The term “aza-cyclic amine” means an amino group appended to the aza-cycle, as defined herein. Representative examples of aza-cyclic amine include, but are not limited to azetidin-3-aminopyrrolidin-3-amine, piperidin-3-amine, and azepan-3-amine.

The term “carbonyl” means a —C(O)— group.

The term “carboxy” means a —CO₂H group.

The term “carboxyalkyl” means a carboxy group appended to the parent molecular moiety through an alkyl group, as defined herein. Representative examples of carboxyalkyl include, but are not limited to, carboxymethyl, 2-carboxyethyl, and 3-carboxypropyl.

The terms “comprise”, “comprises” and “comprising” are transitional terms, which are synonymous with “including,” “containing,” or “characterized by,” and are inclusive or open-ended and do not exclude additional, unrecited elements or method steps.

The term, “concurrently administering” or “concurrent administration” refers to administering, or the administration of, respectively, an α4β2 receptor ligand to a patient, who has been prescribed (or has consumed) at least one α4β2 PAM, at an appropriate time so that the patient's symptoms may subside. This may mean simultaneous administration of an α4β2 PAM and an α4β2 receptor ligand, or administration of the medications at different, but appropriate times.

The term “cyano” means a —CN group.

The term “cyanoalkyl” means a cyano group appended to the parent molecular moiety through an alkyl group, as defined herein. Representative examples of cyanoalkyl include, but are not limited to, cyanomethyl, 2-cyanoethyl, and 3-cyanopropyl.

The term “cycloalkyl” means a monocyclic, bicyclic, or tricyclic ring system. Monocyclic ring systems are exemplified by a saturated cyclic hydrocarbon group containing from 3 to 8 carbon atoms. Examples of monocyclic ring-systems include cyclopropyl, cyclobutyl, cyclopentyl, cyclohexyl, cycloheptyl, and cyclooctyl. Bicyclic ring systems are exemplified by a bridged monocyclic ring system in which two adjacent or non-adjacent carbon atoms of the monocyclic ring are linked by an alkylene bridge of between one and three additional carbon atoms. Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Tricyclic ring systems are exemplified by a bicyclic ring system in which two non-adjacent carbon atoms of the bicyclic ring are linked by a bond or an alkylene bridge of between one and three carbon atoms. Representative examples of tricyclic-ring systems include, but are not limited to, tricyclo[3.3.1.0^{3,7}]nonane and tricyclo[3.3.1.1^{3,7}]decane (adamantane).

The cycloalkyl groups of the invention are optionally substituted with 1, 2, 3, 4 or 5 substituents selected from the group consisting of alkenyl, alkoxy, alkoxyalkoxy, alkoxyalkyl, alkoxycarbonyl, alkoxysulfonyl, alkyl, alkylcarbonyl, alkylcarbonyloxy, alkylsulfonyl, alkylthio, alkylthioalkyl, alkynyl, carboxy, cyano, formyl, haloalkoxy, haloalkyl, halogen, hydroxy, hydroxyalkyl, mercapto, oxo, —NZ¹Z², and (NZ³Z⁴)carbonyl.

The term “formyl” means a —C(O)H group.

The term “formylalkyl” means a formyl group appended to the parent molecular moiety through an alkyl group, as defined herein. Representative examples of formylalkyl include, but are not limited to, formylmethyl and 2-formylethyl.

The term “halo” or “halogen” means —Cl, —Br, —I or —F.

The term “haloalkoxy” means at least one halogen appended to the parent molecular moiety through an alkoxy group, as defined herein. Representative examples of haloalkoxy include, but are not limited to, chloromethoxy, 2-fluoroethoxy, trifluoromethoxy, and pentafluoroethoxy.

The term “haloalkyl” means at least one halogen appended to the parent molecular moiety through an alkyl group, as defined herein. Representative examples of haloalkyl include, but are not limited to, chloromethyl, dichloromethyl, trichloromethyl, fluoromethyl, difluoromethyl, trifluoromethyl, 2-fluoroethyl, pentafluoroethyl, and 2-chloro-3-fluoropentyl.

The term “heteroaryl” means a monocyclic heteroaryl or a bicyclic heteroaryl. The monocyclic heteroaryl is a 5- or 6-membered ring that contains at least one heteroatom selected from the group consisting of nitrogen, oxygen and sulfur. The 5-membered ring contains two double bonds and the 6-membered ring contains three double bonds. The 5- or 6-membered heteroaryl is connected to the parent molecular moiety through any carbon atom or any substitutable nitrogen atom contained within the heteroaryl, provided that proper valance is maintained. Representative examples of monocyclic heteroaryl include, but are not limited to, furyl, imidazolyl, isoxazolyl, isothiazolyl, oxadiazolyl, oxazolyl, pyridin-3-yl, pyridazinyl, pyrimidinyl, pyrazinyl, pyrazolyl, pyrrolyl, tetrazolyl, thiadiazolyl, thiazolyl, triazolyl, and triazinyl. The bicyclic heteroaryl consists of a monocyclic heteroaryl fused to a phenyl, or a monocyclic heteroaryl fused to a cycloalkyl, or a monocyclic heteroaryl fused to a cycloalkenyl, or a monocyclic heteroaryl fused to a monocyclic heteroaryl. The bicyclic heteroaryl is connected to the parent molecular moiety through any carbon atom or any substitutable nitrogen atom contained within the bicyclic heteroaryl, provided that proper valance is maintained. Representative examples of bicyclic heteroaryl include, but are not limited to, azaindolyl, benzimidazolyl, benzofuranyl, benzoxadiazolyl, benzoisoxazole, benzoisothiazole, benzooxazole, 1,3-benzothiazolyl, benzothiophenyl, cinnolinyl, furopyridine, indolyl, indazolyl, isobenzofuran, isoindolyl, isoquinolinyl, naphthyridinyl, oxazolopyridine, quinolinyl, quinoxalinyl and thienopyridinyl.

The heteroaryl groups of the invention are optionally substituted with 1,2,3 or 4 substituents independently selected from the group consisting of alkenyl, alkoxy, alkoxyalkoxy, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylthio, alkylthioalkyl, alkynyl, carboxy, carboxyalkyl, cyano, cyanoalkyl, formyl, haloalkoxy, haloalkyl, halogen, hydroxy, hydroxyalkyl, mercapto, and nitro. Heteroaryl groups of the invention that are substituted with a hydroxy group may be present as tautomers. The heteroaryl groups of the invention encompass all tautomers including non-aromatic tautomers.

The term “heterocycle” or “heterocyclic” means a monocyclic heterocycle, a bicyclic heterocycle or a tricyclic heterocycle. The monocyclic heterocycle is a 3-, 4-, 5-, 6- or 7-membered ring containing at least one heteroatom independently selected from the group consisting of O, N, and S. The 3- or 4-membered ring contains 1 heteroatom selected from the group consisting of O, N and S. The 5-membered ring contains zero or one double bond and one, two or three heteroatoms selected from the group consisting of O, N and S. The 6- or 7-membered ring contains zero, one or two double bonds and one, two, or three heteroatoms selected from the group consisting of O, N and S. The monocyclic heterocycle is connected to the parent molecular moiety through any carbon atom or any nitrogen atom contained within the monocyclic heterocycle. Representative examples of monocyclic heterocycle include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazohinyl, thiazolidinyl, thiomorpholinyl, 1,1-dioxidothiomorpholinyl (thiomorpholine sulfone), thiopyranyl, and trithianyl. The bicyclic heterocycle is a 5- or 6-membered monocyclic heterocycle fused to a phenyl group, or a 5- or 6-membered monocyclic heterocycle fused to a cycloalkyl, or a 5- or 6-membered monocyclic heterocycle fused to a cycloalkenyl, or a 5- or 6-membered monocyclic heterocycle fused to a monocyclic heterocycle. The bicyclic heterocycle is connected to the parent molecular moiety through any carbon atom or any nitrogen atom contained within the bicyclic heterocycle. Representative examples of bicyclic heterocycle include, but are not limited to, 1,3-benzodioxolyl, 1,3-benzodithiolyl, 2,3-dihydro-1,4-benzodioxinyl, benzodioxolyl, 2,3-dihydro-1-benzofuranyl, 2,3-dihydro-1-benzothienyl, chromenyl and 1,2,3,4-tetrahydroquinolinyl. The tricyclic heterocycle is a bicyclic heterocycle fused to a phenyl, or a bicyclic heterocycle fused to a cycloalkyl, or a bicyclic heterocycle fused to a cycloalkenyl, or a bicyclic heterocycle fused to a monocyclic heterocycle. The tricyclic heterocycle is connected to the parent molecular moiety through any carbon atom or any nitrogen atom contained within the tricyclic heterocycle. Representative examples of tricyclic heterocycle include, but are not limited to, 2,3,4,4a,9,9a-hexahydro-1H-carbazolyl, 5a,6,7,8,9,9a-hexahydrodibenzo[b,d]furanyl, and 5a,6,7,8,9,9a-hexahydrodibenzo[b,d]thienyl.

The heterocycles of this invention are optionally substituted with 1, 2, 3 or 4 substituents independently selected from the group consisting of alkenyl, alkoxy, alkoxyalkoxy, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylthio, alkylthioalkyl, alkynyl, carboxy, carboxyalkyl, cyano, cyanoalkyl, formyl, haloalkoxy, haloalkyl, halogen, hydroxy, hydroxyalkyl, nitro, mercapto, oxo, —NZ¹Z² and (NZ³Z⁴)carbonyl.

The term “hydroxy” or “hydroxyl” means an —OH group.

The term “hydroxyalkyl” means at least one hydroxy group is appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “mammal” includes humans and animals, such as cats, dogs, swine, cattle, horses, and the like.

The term “mercapto” means a —SH group.

The term “nitro” means a —NO₂ group.

The term “NZ¹Z²” means two groups, Z¹ and Z², which are appended to the parent molecular moiety through a nitrogen atom. Z¹ and Z² are each independently selected from the group consisting of hydrogen, alkyl, alkylcarbonyl, alkoxycarbonyl, aryl, arylalkyl, and formyl. In certain instances within the invention, Z¹ and Z² taken together with the nitrogen atom to which they are attached form a heterocyclic ring. Representative examples of NZ₁Z₂ include, but are not limited to, amino, methylamino, acetylamino, acetylmethylamino, phenylamino, benzylamino, azetidinyl, pyrrolidinyl and piperidinyl.

The term “NZ³Z⁴” means two groups, Z³ and Z⁴, which are appended to the parent molecular moiety through a nitrogen atom. Z³ and Z⁴ are each independently selected from the group consisting of hydrogen, alkyl, aryl and arylalkyl. Representative examples of NZ³Z⁴ include, but are not limited to, amino, methylamino, phenylamino and benzylamino.

The term “(NZ³Z⁴)carbonyl” means a NZ³Z⁴ group appended to the parent molecular moiety through a carbonyl group, as defined herein. Representative examples of (NZ³Z⁴)carbonyl include, but are not limited to, aminocarbonyl, (methylamino)carbonyl, (dimethylamino)carbonyl, and (ethylmethylamino)carbonyl.

The term “oxo” means a ═O moiety.

The term “parenterally” refers to modes of administration, including intravenous, intramuscular, intraperitoneal, intrasternal, subcutaneous, intraarticular injection, and infusion.

The term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol; and phosphate buffer solutions; as well as other non-toxic compatible lubricants such as sodium lauryl sulfate and magnesium stearate; as well as coloring agents, releasing agents, coating agents, sweetening, flavoring and perfuming agents, preservatives and antioxidants can also be present in the composition, according to the judgment of one skilled in the art of formulations.

The term “pharmaceutically acceptable ester” or “ester” refers to esters of compounds of the invention which hydrolyze in vivo and include those that break down readily in the human body to leave the parent compound or a salt thereof. Examples of pharmaceutically acceptable, non-toxic esters of the invention include, but are not limited to, C₁-to-C₆ alkyl esters and C₅-to-C₇ cycloalkyl esters. Esters of the compounds of formula (I) can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” or “amide” refers to non-toxic amides of the invention derived from ammonia, primary C₁-to-C₃ alkyl amines, primary C₄-to-C₆ alkyl amines, secondary C₁-to-C₂ dialkyl amines and secondary C₃-to-C₆ dialkyl amines. In the case of secondary amines, the amine can also be in the form of a 5- or 6-membered heterocycle containing one nitrogen atom. Amides of the compounds of formula (I) can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the invention in the form of a pharmaceutically acceptable prodrug.

The term “pharmaceutically acceptable prodrug” or “prodrug” represents those prodrugs of the compounds of the invention which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the invention can be rapidly transformed in vivo to a parent compound of formula (I), for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

The term “pharmaceutically acceptable salts” include salts and zwitterions of compounds of formula (I) which are, within the scope of sound medical judgment, suitable for use in contact with the tissues of humans and lower animals without undue toxicity, irritation, allergic response, and the like, are commensurate with a reasonable benefit/risk ratio, and are effective for their intended use. Pharmaceutically acceptable salts are well-known in the art. The salts can be prepared in situ during the final isolation and purification of the compounds of the invention, or separately by reacting a free base function with a suitable organic acid.

Representative acid addition salts include, but are not limited to acetate, adipate, alginate, citrate, aspartate, benzoate, benzenesulfonate, bisulfate, butyrate, camphorate, camphorsulfonate, citrate, digluconate, ethanesulfonate, glycerophosphate, heptanoate, hexanoate, fumarate, hydrochloride, hydrobromide, hydroiodide, hydroxybutyrate, 2-hydroxyethanesulfonate (isethionate), lactate, malate, maleate, methanesulfonate, nicotinate, 2-naphthalenesulfonate, oxalate, pamoate, pectinate, persulfate, phenylacetate, 3-phenylpropionate, picrate, pivalate, propionate, salicylate, succinate, sulfate, tartrate, thiocyanate, phosphate, glutamate, carbonate, p-toluenesulfonate, and undecanoate.

Also, the basic nitrogen-containing groups can be quaternized with agents as alkyl halides such as methyl, ethyl, propyl, butyl, decyl, lauryl, myristyl and stearyl chlorides, bromides and iodides; dialkyl sulfates such as dimethyl, diethyl, dibutyl and diamyl sulfates; arylalkyl halides such as benzyl and phenethyl bromides and others. Water or oil-soluble or dispersible products are thereby obtained.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium, and aluminum salts, and the like, and nontoxic quaternary ammonia and amine cations including ammonium, tetramethylammonium, tetraethylammonium, methylamine, dimethylamine, trimethylamine, triethylamine, diethylamine, ethylamine and the such. Other representative organic amines useful for the formation of base addition salts include ethylenediamine, ethanolamine, diethanolamine, piperidine, and piperazine.

The term “positive allostertc modulator” or PAM means a compound that enhances activity of an endogenous, or naturally occurring, ligand, such as but not limited to ACh, or an exogenously administered agonist. Although typically it may be recognized that an asterisk is used to indicate that the exact subunit composition of a receptor is uncertain, for example α4β2* indicates a receptor that contains the α4 and β2 subunits proteins in combination with other subunits.

The term “sulfinyl” means a —S(O)— group.

The term “sulfonyl” means a —SO₂— group.

The term “tautomer” means a proton shift from one atom of a compound to another atom of the same compound wherein two or more structurally distinct compounds are in equilibrium with each other.

The phrase “therapeutically effective amount” of the compound of the invention means a sufficient amount of the compound to treat disorders, at a reasonable benefit/risk ratio applicable to any medical treatment.

### COMPOUNDS OF THE INVENTION

Various embodiments of the invention comprise compounds of formula (I), novel intermediates thereof, and pharmaceutically acceptable salts, amides, esters, isomers, and prodrugs thereof.

An embodiment of the invention is compounds having the formula (I)

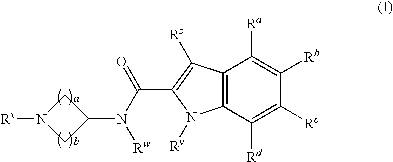

wherein a and b are independently 1, 2, 3, or 4; R^{a}, R^{b}, R^{c} and R^{d} are independently hydrogen, alkyl, aryl, cyano, halogen, haloalkyl, heteroaryl, NR¹R², nitro, OR³; SR¹, or SO₂R¹; or R^{a}, R^{b}, and the carbon atoms to which they are attached taken together form a monocyclic aryl or monocyclic heteroaryl; R¹ and R² are independently hydrogen, alkyl, arylalkyl, or cycloalkyl; R³ is hydrogen, alkyl, arylalkyl, cycloalkyl, or haloalkyl; R^{w} is hydrogen or alkyl; R^{x} and R^{y} are independently hydrogen, alkyl, or cycloalkyl; and R^{z} is hydrogen, alkyl, aryl, or heteroaryl.

In another embodiment of the invention, a is 1, 2, 3, or 4, and b is 1.

In one embodiment of the invention, a and b are each 1.

In another embodiment of the invention, a and b are each 2.

In an additional embodiment of the invention, a is 2 and b is 1.

In another embodiment of the invention, a is 3 and b is 1.

In a further embodiment of the invention, a is 4 and b is 1.

In another embodiment of the invention, R^{w}, R^{x}, and R^{y} are independently hydrogen or alkyl; and R^{z} is hydrogen, alkyl, aryl or heteroaryl.

In another embodiment of the invention, R^{w} is hydrogen, R^{x} is alkyl, R^{y} is hydrogen, and R^{z} is alkyl.

In another embodiment of the invention, R^{w} is hydrogen, R^{x} is alkyl, R^{y} is hydrogen, and R^{z} is hydrogen.

In another embodiment of the invention, R^{w}, R^{x}, R^{y}, and R^{z} are each hydrogen.

In another embodiment of the invention, R^{w}, R^{x} and R^{y} are hydrogen, and R^{z} is alkyl.

In another embodiment of the invention, R^{w}, R^{x} and R^{y} are hydrogen, and R^{z} is aryl.

In another embodiment of the invention, R^{w} is alkyl, R^{x} is alkyl, R^{y} is hydrogen, and R^{z} is alkyl.

In another embodiment of the invention, R^{w} is alkyl, R^{x} is hydrogen, R^{y} is hydrogen, and R^{z} is alkyl.

In a further embodiment of the invention, R^{x} is hydrogen or methyl, and R^{y} is hydrogen.

In another embodiment of the invention, one of R^{a}, R^{b}, R^{c} and R^{d} are each hydrogen.

In another embodiment of the invention, one of R^{a}, R^{b}, R^{c} and R^{d} is alkyl aryl, OR³, halogen, SR¹, NR¹R², nitro, or SO₂R¹ and the others are hydrogen.

In another embodiment of the invention, any two of R^{a}, R^{b}, R^{c} and R^{d} are alkyl and the others are hydrogen.

In another embodiment of the invention, any two of R^{a}, R^{b}, R^{c} and R^{d} are halogen and the others are hydrogen.

In another embodiment of the invention, any two of R^{a}, R^{b}, R^{c} and R^{d} are OR³ and the others are hydrogen.

In another embodiment of the invention, one of R^{a}, R^{b}, R^{c} and R^{d} is halogen; another of R^{a}, R^{b}, R^{c} and R^{d} is alkyl; and the other two are hydrogen.

In another embodiment of the invention, any three of R^{a}, R^{b}, R^{c} and R^{d} are alkyl and the other is hydrogen.

In a further embodiment of the invention, R^{a} and R^{b} taken together with the carbon atoms to which they are attached form a fused phenyl ring and R^{c} and R^{d} are hydrogen.

Another embodiments of the invention is a compound of formula (I), selected form the group of compounds exemplified in examples 1-160 below.

Another embodiment of the invention is a compound of formula (I), or a pharmaceutically acceptable salt thereof, selected from the group of compounds exemplified in Examples 1-4, 7, 10, 13, 14, 19, 20, 22, 24-103, 106, 110, 111, 158, and 159 below.

Another embodiment of the invention is a compound of formula (I) selected from the group of compounds exemplified in Examples 5, 6, 7, 8, 11, 12, 15, 18, 21, 23, 104, 105, 107-109, 112-153, 156, 157, and 160 below.

Another embodiment of the invention is a compound of formula (I), selected from the group of compounds exemplified in Examples 16 and 17 below.

Another embodiment of the invention is (R)-3,4,7-trimethyl-N-(pyrrolidin-3-yl)-1H-indole-2-carboxamide.

Another embodiment of the invention is (S)-3,4,7-trimethyl-N-(pyrrolidin-3-yl)-1H-indole-2-carboxamide.

Another embodiment of the invention is (S)-4,6-dichloro-N-(pyrrolidin-3-yl)-1H-indole-2-carboxamide.

Another embodiment of the invention is (R)-4,6-dimethyl-N-(pyrrolidin-3-yl)-1H-indole-2-carboxamide.

Another embodiment of the invention is (R)-4,6-dimethyl-N-(piperidin-3-yl)-1H-indole-2-carboxamide.

Another embodiment of the invention is (S)-3,4,7-trimethyl-N-(piperidin-3-yl)-1H-indole-2-carboxamide.

Another embodiment of the invention is (R)-4,6-dimethyl-N-(1-methylpyrrolidin-3-yl)-1H-indole-2-carboxamide.

Another embodiment of the invention is (R)-4,6-dimethyl-N-(1-methylpiperidin-3-yl)-1H-indole-2-carboxamide.

Another embodiment of the invention is (S)-3,4,7-trimethyl-N-(1-methylpiperidin-3-yl)-1H-indole-2-carboxamide.

Another embodiment of the invention is (R)-6-tert-butyl-N-(pyrrolidin-3-yl)-1H-indole-2-carboxamide.

Another embodiment of the invention is (R)-6-tert-butyl-N-(piperidin-3-yl)-1H-indole-2-carboxamide.

Another embodiment of the invention is (R)-6-phenyl-N-(piperidin-3-yl)-1H-indole-2-carboxamide.

Another embodiment of the invention is (R)-6-phenyl-N-(pyrrolidin-3-yl)-1H-indole-2-carboxamide.

Another embodiment of the invention is (R)-4,5-dimethyl-N-(pyrrolidin-3-yl)-1H-indole-2-carboxamide.

Another embodiment of the invention is (R)-4,5-dimethyl-N-(piperidin-3-yl)-1H-indole-2-carboxamide.

Another embodiment of the invention is N-(azepan-3-yl)-4,6-dichloro-1H-indole-2-carboxamide.

Another embodiment of the invention is N-(azepan-3-yl)-3,4,7-trimethyl-1H-indole-2-carboxamide.

Another embodiment of the invention is (R)-6-methoxy-N-(piperidin-3-yl)-1H-indole-2-carboxamide.

Another embodiment of the invention is (R)-6-methoxy-N-(pyrrolidin-3-yl)-1H-indole-2-carboxamide.

Another embodiment of the invention is (R)-6-chloro-7-methyl-N-(pyrrolidin-3-yl)-1H-indole-2-carboxamide.

Another embodiment of the invention is (R)-6-chloro-7-methyl-N-(piperidin-3-yl)-1H-indole-2-carboxamide.

Another embodiment of the invention is (S)-6-chloro-7-methyl-N-(pyrrolidin-3-yl)-1H-indole-2-carboxamide.

Another embodiment of the invention is (S)-6-chloro-7-methyl-N-(piperidin-3-yl)-1H-indole-2-carboxamide.

Another embodiment of the invention is (R)-5-bromo-N-(pyrrolidin-3-yl)-1H-indole-2-carboxamide.

Another embodiment of the invention is (R)-1-methyl-N-(pyrrolidin-3-yl)-1H-indole-2-carboxamide.

Another embodiment of the invention is (R)-5-bromo-N-(1-methylpyrrolidin-3-yl)-1H-indole-2-carboxamide.

Another embodiment of the invention is (R)-4,6-dichloro-N-(pyrrolidin-3-yl)-1H-1-indole-2-carboxamide.

Another embodiment of the invention is (R)-4-methoxy-N-(pyrrolidin-3-yl)-1H-indole-2-carboxamide.

Another embodiment of the invention is (R)-5-chloro-N-(pyrrolidin-3-yl)-1H-indole-2-carboxamide.

Another embodiment of the invention is (R)-4,6-dichloro-N-(1-methylpyrrolidin-3-yl)-1H-indole-2-carboxamide.

Another embodiment of the invention is (R)-5-chloro-N-(1-methylpyrrolidin-3-yl)-1H-indole-2-carboxamide.

Another embodiment of the invention is (R)-4-methoxy-N-(1-methylpyrrolidin-3-yl)-1H-indole-2-carboxamide.

Another embodiment of the invention is (R)-N,3,4,7-tetramethyl-N-(pyrrolidin-3-yl)-1H-indole-2-carboxamide.

Another embodiment of the invention is (R)-N,3,4,7-tetramethyl-N-(1-methylpyrrolidin-3-yl)-1H-indole-2-carboxamide.

Another embodiment of the invention is (S)-5-fluoro-N-(pyrrolidin-3-yl)-1H-indole-2-carboxamide.

Another embodiment of the invention is (S)-5-methoxy-N-(pyrrolidin-3-yl)-1H-indole-2-carboxamide.

Another embodiment of the invention is (S)-5-fluoro-N-(1-methylpyrrolidin-3-yl)-1H-indole-2-carboxamide.

Another embodiment of the invention is (S)-5-methoxy-N-(1-methylpyrrolidin-3-yl)-1H-indole-2-carboxamide.

Another embodiment of the invention is (S)-5,6-dimethoxy-N-pyrrolidin-3-yl)-1H-indole-2-carboxamide.

Another embodiment of the invention is (S)-4,6-dichloro-N-(1-methylpyrrolidin-3-yl)-1H-indole-2-carboxamide.

Another embodiment of the invention is (R)-N-(pyrrolidin-3-yl)-3H-benzo[e]indole-2-carboxamide.

Another embodiment of the invention is (S)-N-(pyrrolidin-3-yl)-3H-benzo[e]indole-2-carboxamide.

Another embodiment of the invention is (R)-4-(difluoromethoxy)-N-(pyrrolidin-3-yl)-1H-indole-2-carboxamide.

Another embodiment of the invention is (S)-4-(difluoromethoxy)-N-(pyrrolidin-3-yl)-1H-indole-2-carboxamide.

Another embodiment of the invention is (S)-7-fluoro-N-(pyrrolidin-3-yl)-1H-indole-2-carboxamide.

Another embodiment of the invention is (S)-6-tert-butyl-N-(pyrrolidin-3-yl)-1H-indole-2-carboxamide.

Another embodiment of the invention is (R)-4-fluoro-N-(pyrrolidin-3-yl)-1H-indole-2-carboxamide.

Another embodiment of the invention is (S)-4-fluoro-N-(pyrrolidin-3-yl)-1H-indole-2-carboxamide.

Another embodiment of the invention is (R)-6-(methylthio)-N-(pyrrolidin-3-yl)-1H-indole-2-carboxamide.

Another embodiment of the invention is (S)-6-(methylthio)-N-(pyrrolidin-3-yl)-1H-indole-2-carboxamide:

Another embodiment of the invention is (R)-3,5-dimethyl-N-(pyrrolidin-3-yl)-1H-indole-2-carboxamide.

Another embodiment of the invention is (S)-3,5-dimethyl-N-(pyrrolidin-3-yl)-1H-indole-2-carboxamide.

Another embodiment of the invention is (R)-4-chloro-N-(pyrrolidin-3-yl)-1H-indole-2-carboxamide.

Another embodiment of the invention is (S)-4-chloro-N-(pyrrolidin-3-yl)-1H-indole-2-carboxamide.

Another embodiment of the invention is (R)-4-methyl-N-(pyrrolidin-3-yl)-1H-indole-2-carboxamide.

Another embodiment of the invention is (S)-4-methyl-N-pyrrolidin-3-yl)-1H-indole-2-carboxamide.

Another embodiment of the invention is (R)-3-methyl-N-(pyrrolidin-3-yl)-1H-indole-2-carboxamide.

Another embodiment of the invention is (S)-3-methyl-N-(pyrrolidin-3-yl)-1-indole-2-carboxamide.

Another embodiment of the invention is (R)-N-(1-methylpyrrolidin-3-yl)-3H-benzo[e]indole-2-carboxamide.

Another embodiment of the invention is (R)-6-chloro-N-(pyrrolidin-3-yl)-1H-indole-2-carboxamide.

Another embodiment of the invention is (S)-6-chloro-N-(pyrrolidin-3-yl)-1H-indole-2-carboxamide.

Another embodiment of the invention is (S)-N-(1-methylpyrrolidin-3-yl)-3H-benzo[e]indole-2-carboxamide.

Another embodiment of the invention is (R)-6-bromo-N-(pyrrolidin-3-yl)-1H-indole-2-carboxamide.

Another embodiment of the invention is (S)-6-bromo-N-(pyrrolidin-3-yl)-1H-indole-2-carboxamide.

Another embodiment of the invention is (S)-7-methyl-N-(pyrrolidin-3-yl)-1H-indole-2-carboxamide.

Another embodiment of the invention is (R)-7-methyl-N-(pyrrolidin-3-yl)-1H-indole-2-carboxamide.

Another embodiment of the invention is (R)-4-(difluoromethoxy)-N-(1-methylpyrrolidin-3-yl)-1H-indole-2-carboxamide.

Another embodiment of the invention is (S)-4-(difluoromethoxy)-N-(1-methylpyrrolidin-3-yl)-1H-indole-2-carboxamide.

Another embodiment of the invention is (S)-5-chloro-N-(pyrrolidin-3-yl)-1H-indole-2-carboxamide.

Another embodiment of the invention is (R)-3-methyl-N-(1-methylpyrrolidin-3-yl)-1H-indole-2-carboxamide.

Another embodiment of the invention is (S)-3-methyl-N-(1-methylpyrrolidin-3-yl)-1H-indole-2-carboxamide.

Another embodiment of the invention is (R)-4-chloro-N-(1-methylpyrrolidin-3-yl)-1H-indole-2-carboxamide.

Another embodiment of the invention is (S)-4-chloro-N-(1-methylpyrrolidin-3-yl)-1H-indole-2-carboxamide.

Another embodiment of the invention is (R)-6-chloro-N-(1-methylpyrrolidin-3-yl)-1H-indole-2-carboxamide.

Another embodiment of the invention is (R)-5-methyl-N-(pyrrolidin-3-yl)-1H-indole-2-carboxamide.

Another embodiment of the invention is (S)-5-bromo-N-(pyrrolidin-3-yl)-1H-indole-2-carboxamide.

Another embodiment of the invention is (R)-4,6,7-trimethyl-N-(pyrrolidin-3-yl)-1H-indole-2-carboxamide.

Another embodiment of the invention is (S)-4,6,7-trimethyl-N-(pyrrolidin-3-yl)-1H-indole-2-carboxamide.

Another embodiment of the invention is (R)-4,7-dimethoxy-N-(pyrrolidin-3-yl)-1H-indole-2-carboxamide.

Another embodiment of the invention is (S)-6-chloro-N-(1-methylpyrrolidin-3-yl)-1H-indole-2-carboxamide.

Another embodiment of the invention is (S)-4,7-dimethoxy-N-(pyrrolidin-3-yl)-1H-indole-2-carboxamide.

Another embodiment of the invention is (R)-6-bromo-N-(1-methylpyrrolidin-3-yl)-1H-indole-2-carboxamide.

Another embodiment of the invention is (R)-7-methyl-N-(1-methylpyrrolidin-3-yl)-1H-indole-2-carboxamide.

Another embodiment of the invention is (S)-7-methyl-N-(1-methylpyrrolidin-3-yl)-1H-indole-2-carboxamide.

Another embodiment of the invention is (R)-5-methyl-N-(1-methylpyrrolidin-3-yl)-1H-indole-2-carboxamide.

Another embodiment of the invention is (S)-5-bromo-N-(1-methylpyrrolidin-3-yl)-1H-indole-2-carboxamide.

Another embodiment of the invention is (R)-4,6,7-trimethyl-N-(1-methylpyrrolidin-3-yl)-1H-indole-2-carboxamide.

Another embodiment of the invention is (S)-4,6,7-trimethyl-N-(1-methylpyrrolidin-3-yl)-1H-indole-2-carboxamide.

Another embodiment of the invention is (R)-4,7-dimethoxy-N-(1-methylpyrrolidin-3-yl)-1H-indole-2-carboxamide.

Another embodiment of the invention is (S)-4,7-dimethoxy-N-(1-methylpyrrolidin-3-yl)-1H-indole-2-carboxamide.

Another embodiment of the invention is (R)-4,7-dimethyl-N-(pyrrolidin-3-yl)-1H-indole-2-carboxamide.

Another embodiment of the invention is (R)-6-bromo-4-fluoro-N-(pyrrolidin-3-yl)-1H-indole-2-carboxamide.

Another embodiment of the invention is (R)-6-fluoro-7-methyl-N-(pyrrolidin-3-yl)-1H-indole-2-carboxamide.

Another embodiment of the invention is (R)-4,7-dimethyl-N-(1-methylpyrrolidin-3-yl)-1H-indole-2-carboxamide.

Another embodiment of the invention is (R)-6-bromo-4-fluoro-N-(1-methylpyrrolidin-3-yl)-1H-indole-2-carboxamide.

Another embodiment of the invention is (R)-5-trifluoromethoxy-N-(pyrrolidin-3-yl)-1H-indole-2-carboxamide.

Another embodiment of the invention is (R)-5,7-dimethoxy-N-(pyrrolidin-3-yl)-1H-indole-2-carboxamide.

Another embodiment of the invention is (R)-6-(dimethylamino)-N-(pyrrolidin-3-yl)-1H-indole-2-carboxamide.

Another embodiment of the invention is (R)-5,6-dimethoxy-N-(pyrrolidin-3-yl)-1H-indole-2-carboxamide.

Another embodiment of the invention is (R)-N-(pyrrolidin-3-yl)-1H-indole-2-carboxamide.

Another embodiment of the invention is (R)-5-chloro-3-methyl-N-(pyrrolidin-3-yl)-1H-indole-2-carboxamide.

Another embodiment of the invention is (R)-6-(dimethylamino)-N-(1-methylpyrrolidin-3-yl)-1H-indole-2-carboxamide.

Another embodiment of the invention is (R)-5,6-dimethoxy-N-(1-methylpyrrolidin-3-yl)-1H-indole-2-carboxamide.

Another embodiment of the invention is (R)-7-nitro-N-(piperidin-3-yl)-1H-indole-2-carboxamide.

Another embodiment of the invention is (R)-6-(dimethylamino)-N-(piperidin-3-yl)-1H-indole-2-carboxamide.

Another embodiment of the invention is (R)-4,6-dimethoxy-N-(pyrrolidin-3-yl)-1H-indole-2-carboxamide.

Another embodiment of the invention is (R)-4,6-dimethoxy-N-(piperidin-3-yl)-1H-indole-2-carboxamide.

Another embodiment of the invention is (R)-3,4,7-trimethyl-N-piperidin-3-yl)-1H-indole-2-carboxamide.

Another embodiment of the invention is (R)-3,4,7-trimethyl-N-(1-methylpiperidin-3-yl)-1H-indole-2-carboxamide.

Another embodiment of the invention is (S)-3,4,7-trimethyl-N-(1-methylpyrrolidin-3-yl)-1H-indole-2-carboxamide.

Another embodiment of the invention is (R)-3,4,7-trimethyl-N-(1-methylpyrrolidin-3-yl)-1H-indole-2-carboxamide.

Another embodiment of the invention is (R)-N-(piperidin-3-yl)-1H-indole-2-carboxamide.

Another embodiment of the invention is (R)-N-(1-methylpiperidin-3-yl)-1H-indole-2-carboxamide.

Another embodiment of the invention is (R)-4,6-dichloro-N-(piperidin-3-yl)-1H-indole-2-carboxamide.

Another embodiment of the invention is (R)-4,6-dichloro-N-(1-methylpiperidin-3-yl)-1H-indole-2-carboxamide.

Another embodiment of the invention is (R)-4,6-dichloro-N-(piperidin-3-yl)-1H-indole-2-carboxamide.

Another embodiment of the invention is (R)-N-(piperidin-3-yl)-5-(trifluoromethoxy)-1H-indole-2-carboxamide.

Another embodiment of the invention is (R)-5-(benzyloxy)-N-(piperidin-3-yl)-1H-indole-2-carboxamide.

Another embodiment of the invention is (S)-4,6-dichloro-N-(piperidin-3-yl)-1H-indole-2-carboxamide.

Another embodiment of the invention is (S)-4,6-dichloro-N-(1-methylpiperidin-3-yl)-1H-indole-2-carboxamide.

Another embodiment of the invention is (R)-3-methyl-N-(piperidin-3-yl)-1H-indole-2-carboxamide.

Another embodiment of the invention is (R)-3,5-dimethyl-N-(piperidin-3-yl)-1H-indole-2-carboxamide.

Another embodiment of the invention is (S)-3-methyl-N-(piperidin-3-yl)-1H-indole-2-carboxamide.

Another embodiment of the invention is (S)-3,5-dimethyl-N-(piperidin-3-yl)-1H-indole-2-carboxamide.

Another embodiment of the invention is (R)-3-methyl-N-(1-methylpiperidin-3-yl)-1H-indole-2-carboxamide.

Another embodiment of the invention is (R)-3,5-dimethyl-N-(1-methylpiperidin-3-yl)-1H-indole-2-carboxamide.

Another embodiment of the invention is (S)-3-methyl-N-(1-methylpiperidin-3-yl)-1H-indole-2-carboxamide.

Another embodiment of the invention is (S)-3,5-dimethyl-N-(1-methylpiperidin-3-yl)-1H-indole-2-carboxamide.

Another embodiment of the invention is (S)-6-tert-butyl-N-(piperidin-3-yl)-1H-indole-2-carboxamide.

Another embodiment of the invention is (R)-4,6,7-trimethyl-N-(piperidin-3-yl)-1H-indole-2-carboxamide.

Another embodiment of the invention is (R)-5-chloro-3-methyl-N-(piperidin-3-yl)-1H-indole-2-carboxamide.

Another embodiment of the invention is (R)-4,6,7-trimethyl-N-(1-methylpiperidin-3-yl)-1H-indole-2-carboxamide.

Another embodiment of the invention is (R)-4,7-dimethyl-N-(piperidin-3-yl)-1H-indole-2-carboxamide.

Another embodiment of the invention is (R)-7-fluoro-N-(piperidin-3-yl)-1H-indole-2-carboxamide.

Another embodiment of the invention is (R)-5-chloro-3-phenyl-N-(piperidin-3-yl)-1H-indole-2-carboxamide.

Another embodiment of the invention is (R)-5,7-dimethoxy-N-(piperidin-3-yl)-1H-indole-2-carboxamide.

Another embodiment of the invention is (R)-4-(difluoromethoxy)-N-(piperidin-3-yl)-1H-indole-2-carboxamide.

Another embodiment of the invention is (R)-4-fluoro-N-(piperidin-3-yl)-1H-indole-2-carboxamide.

Another embodiment of the invention is (R)-7-methyl-3-phenyl-N-(piperidin-3-yl)-1H-indole-2-carboxamide.

Another embodiment of the invention is (R)-7-fluoro-N-(1-methylpiperidin-3-yl)-1H-indole-2-carboxamide.

Another embodiment of the invention is (R)-6-bromo-4-fluoro-N-(piperidin-3-yl)-1H-indole-2-carboxamide.

Another embodiment of the invention is (R)-4-methyl-A-(piperidin-3-yl)-1H-indole-2-carboxamide.

Another embodiment of the invention is (R)-4-chloro-N-(piperidin-3-yl)-1H-indole-2-carboxamide.

Another embodiment of the invention is (R)-6-bromo-N-(piperidin-3-yl)-1H-indole-2-carboxamide.

Another embodiment of the invention is (R)-6-chloro-N-(piperidin-3-yl)-1H-indole-2-carboxamide.

Another embodiment of the invention is (R)-6-ethyl-N-(piperidin-3-yl)-1H-indole-2-carboxamide.

Another embodiment of the invention is (R)-5,7-dimethyl-N-(piperidin-3-yl)-1H-indole-2-carboxamide.

Another embodiment of the invention is (R)-5-fluoro-7-(methylsulfonyl)-N-(piperidin-3-yl)-1H-indole-2-carboxamide.

Another embodiment of the invention is (R)-4,7-dimethoxy-N-(piperidin-3-yl)-1H-indole-2-carboxamide.

Another embodiment of the invention is (R)-6-(methylthio)-N-(piperidin-3-yl)-1H-indole-2-carboxamide.

Another embodiment of the invention is (S)-4,6,7-trimethyl-N-(piperidin-3-yl)-1H-indole-2-carboxamide.

Another embodiment of the invention is (S)-N-(piperidin-3-yl)-1H-indole-2-carboxamide.

Another embodiment of the invention is (S)-7-methyl-N-(piperidin-3-yl)-1H-indole-2-carboxamide.

Another embodiment of the invention is 4,6-dichloro-N-(1-methylazetidin-3-yl)-1H-indole-2-carboxamide.

Another embodiment of the invention is 4,6-dichloro-N-(piperidin-4-yl)-1H-indole-2-carboxamide.

Another embodiment of the invention is (R)-N-(1-methylpiperidin-3-yl)-7-nitro-1H-indole-2-carboxamide.

Another embodiment of the invention is (R)-4,6-dimethoxy-N-(1-methylpiperidin-3-yl)-1H-indole-2-carboxamide.

Another embodiment of the invention is (R)-4,6-dimethoxy-N-(1-methylpyrrolidin-3-yl)-1H-indole-2-carboxamide.

Another embodiment of the invention is (R)-6-fluoro-7-methyl-N-(1-methylpyrrolidin-3-yl)-1H-indole-2-carboxamide.

Another embodiment of the invention is (R)-6-(dimethylamino)-N-(1-methylpiperidin-3-yl)-1H-indole-2-carboxamide.

Compounds of the present invention may exist as stereoisomers wherein, asymmetric or chiral centers are present. These stereoisomers are “R” or “S” depending on the configuration of substituents around the chiral carbon atom. The terms “R” and “S” used herein are configurations as defined in IUPAC 1974 Recommendations for Section E, Fundamental Stereochemistry, Pure Appl. Chem. 1976, 45: 13-30. The present invention contemplates various stereoisomers and mixtures thereof and these are specifically included within the scope of this invention. Stereoisomers include enantiomers and diastereomers, and mixtures of enantiomers or diastereomers. Individual stereoisomers of compounds of the present invention may be prepared synthetically from commercially available starting materials that contain asymmetric or chiral centers or by preparation of racemic mixtures followed by resolution which is well known to those of ordinary skill in the art. These methods of resolution are exemplified by (1) attachment of a mixture of enantiomers to a chiral auxiliary, separation of the resulting mixture of diastercomers by recrystallization or chromatography and liberation of the optically pure product from the auxiliary or (2) direct separation of the mixture of optical enantiomers on chiral chromatographic columns.

Compounds of the invention can exist in radiolabeled or isotope labeled form containing one or more atoms having an atomic mass or mass number different from the atomic mass or mass number most abundantly found in nature. Isotopes of atoms such as hydrogen, carbon, phosphorous, sulfur, fluorine, chlorine, and iodine include, but are not limited to, ²H, ³H, ¹⁴C, ³²P, ³⁵S, ¹⁸F, ³⁶Cl, and ¹²⁵I. Compounds that contain other radioisotopes of these and/or other atoms are within the scope of this invention. In an embodiment of the invention, the isotope-labeled compounds contain deuterium (²H), tritium (³H) or ¹⁴C radioisotopes. Isotope and radiolabeled compounds of this invention can be prepared by the general methods well known to persons having ordinary skill in the art. Such isotope and radiolabeled compounds can be conveniently prepared by carrying out the procedures disclosed in the following Examples and Schemes by substituting a readily available isotope or radiolabeled reagent for a non-labeled reagent. The isotope and radiolabeled compounds of the invention may be used as standards to determine the effectiveness of α4β2 NNR ligands or modulators in the binding assays.

Geometric isomers can exist in the present compounds. The invention contemplates the various geometric isomers and mixtures thereof resulting from the disposition of substituents around a carbon-carbon double bond, a carbon nitrogen double bond, a cycloalkyl group, or a heterocycloalkyl group. Substituents around a carbon-carbon or carbon-nitrogen double bond are designated as being of Z or E configuration and substituents around a cycloalkyl or heterocycloalkyl are designated as being of cis or trans configuration.

Within the present invention it is to be understood that compounds disclosed herein can exhibit the phenomenon of tautomerism. Thus, when the formulae drawings within this specification represent one of the possible tautomeric or stereoisomeric forms, it is to be understood that the invention encompasses any tautomeric or stereoisomeric form, and mixtures thereof, and is not to be limited merely to any one tautomeric or stereoisomeric form utilized within the naming of the compounds or formulae drawings.

### Amides, Esters and Prodrugs

Prodrugs are pharmacologically inactive derivatives of an active drug designed to ameliorate some identified, undesirable physical or biological property. The physical properties are usually solubility (too much or not enough lipid or aqueous solubility) or stability related, while problematic biological properties include too rapid metabolism or poor bioavailability which itself may be related to a physicochemical property.

Prodrugs are usually prepared by: a) formation of ester, hemi esters, carbonate esters, nitrate esters, amides, hydroxamic acids, carbamates, imines, Mannich bases, and enamines of the active drug, b) functionalizing the drug with azo, glycoside, peptide, and ether functional groups, c) use of polymers, salts, complexes, phosphoramides, acetals, hemiacetals, and ketal forms of the drug. For example, see Andrejus Korolkovas's, “Essentials of Medicinal Chemistry”. John Wiley-Interscience Publications, John Wiley and Sons, New York (1988), pp. 97-118, which is incorporated in its entirety by reference herein.

Esters can be prepared from substrates of formula (I) containing either a hydroxyl group or a carboxy group by general methods known to persons skilled in the art. The typical reactions of these compounds are substitutions replacing one of the heteroatoms by another atom, for example:

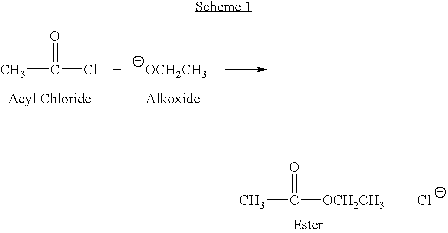

Amides can be prepared from substrates of formula (I) containing either an amino group or a carboxy group in similar fashion. Esters can also react with amines or ammonia to form amides.

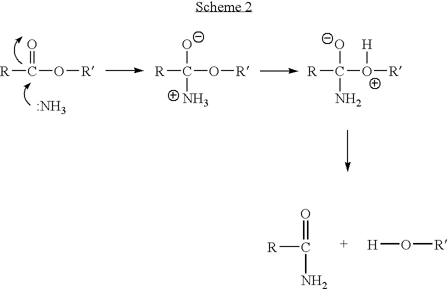

Another way to make amides from compounds of formula (I) is to heat carboxylic acids and amines together.

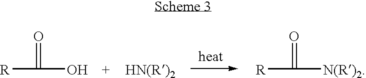

In Schemes 2 and 3, R and R′ are independently substrates of formula (I), alkyl or hydrogen. Various embodiments of the invention of formula (I) that are substrates for prodrugs, and amides include, but are not limited to, Examples 1-6, 10-25, 27-29, 33-36, 39, 41-58, 60-61, 63-66, 69, 75-79, 81, 91-93, 96-101, 104-108, 112, 114, 116-119, 121-124, 129-131, 133-139, 141-153, and 155.

### METHODS OF PREPARING COMPOUNDS OF THE INVENTION

The compounds of the invention can be better understood in connection with the following synthetic schemes and methods which illustrate a means by which the compounds can be prepared.

Abbreviations which have been used in the descriptions of the schemes and the examples that follow are: O-(7-azabenzotriazol-1-yl)-N,N,N′,N′-tetramethyluronium hexafluorophosphate (HATU), t-butoxycarbonyl (Boc), 1-(3-dimethylaminopropyl)-3-ethylcarbodiimide hydrochloride (EDC or EDCI), dimethyl sulfoxide (DMSO), high-pressure liquid chromatography (HPLC), 1-hydroxybenzotriazole hydrate (HOBt), methanol (MeOH), Dulbecco's Modified Eagle's Medium (DMEM), fetal bovine serum (FBS), N-methyl-D-glucamine (NMDG), and 4-(2-hydroxyethyl)-1-piperazineethanesulfonic acid (HEPES).

The compounds of this invention can be prepared according to the synthetic Scheme and/or Examples described. Certain groups can be substituted as described within the scope of this invention as would be known to one skilled in the art. Representative procedures are shown in, but are not limited to, Scheme 4.

As outlined in the Scheme 4, compounds of formula (2), wherein R^{a}, R^{b}, R^{c}, R^{d}, and R^{y} are as defined in formula (I), can react with compounds of formula (1), wherein R^{m} is an alkyl such as but not limited to methyl or ethyl, in a solvent such as tetrahydrofuran or methanol in the presence of a base such as but not limited to sodium methoxide or sodium ethoxide at −10° C. to 0° C. for 1-12 hours to provide compounds of formula (3). Then compounds of formula (3) can be treated with a rhodium catalyst, such as but not limited to rhodium(II) acetate dimer, rhodium(II) trifluoroacetate dimer or rhodium(II) heptafluorobutyrate dimer, in a solvent such as toluene at 30-100° C. for 4-40 hours to give compounds of formula (4) as described by Driver, T. G., et al., J. Am. Chem. Soc., 129: 7500-7501, 2007. Compounds of formula (4) can be hydrolyzed by a base such as sodium hydroxide or potassium hydroxide and then acidified with an acid such as hydrochloric acid to give compounds of formula (5). Some examples of compounds of formula (5) are commercially available. Compounds of formula (5) can be coupled with an aza-cyclic amine of formula (6), wherein a and b are as defined in formula (I) in the presence of a coupling agent, such as but not limited to O-(7-azabenzotriazol-1-yl)-N,N,N′,N′-tetramethyluronium hexafluorophosphate (HATU) or 1-(3-dimethylaminopropyl)-3-ethylcarbodiimide hydrochloride (EDC)/1-hydroxybenzotriazole hydrate (HOBt), in a solvent such as tetrahydrofuran, N,N-dimethylformamide or dichloromethane, to give compounds of formula (7) that can be treated with an acid, such as but not limited to trifluoroacetic acid, hydrochloric acid or p-toluenesulfonic acid, at room temperature to 100° C. to give compounds of formula (8). Compounds of formula (8) can be reacted with an aldehyde of formula (9), wherein R^{x} is defined as in formula (I), in the presence of a reducing agent such as sodium triacetoxyborohydride or sodium cyanoborohydride to give compound of formula (11).

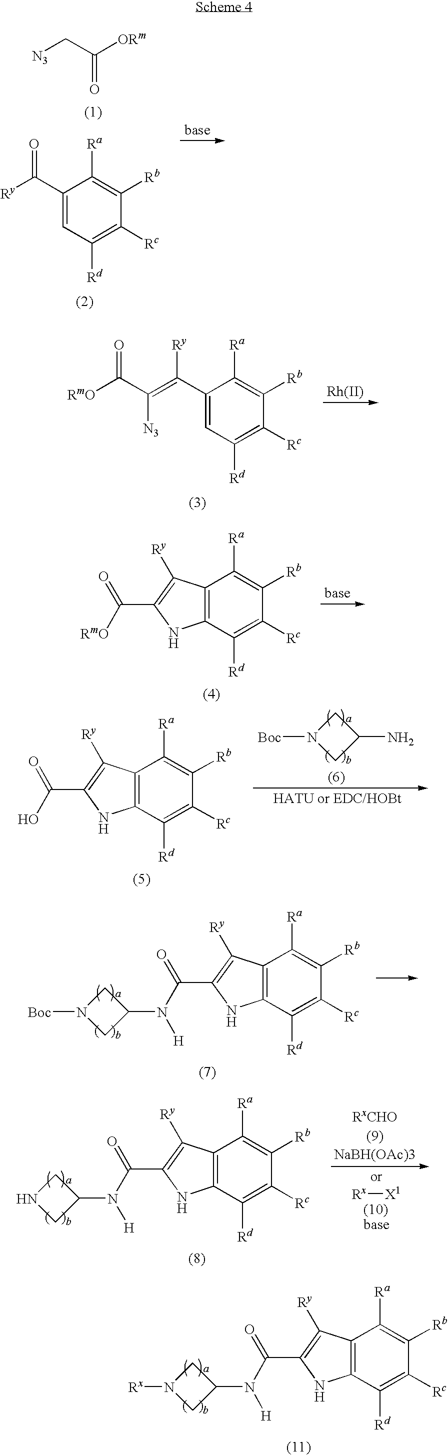

Alternatively, compounds of formula (11) can also be prepared by the reaction of compounds of formula (8) with compounds of formula (10), wherein R^{x} is as defined in formula (I) and X¹ is chlorine, bromine, or iodine, in the presence of a base, such as but not limited to sodium carbonate, potassium carbonate or sodium hydride, in a solvent such as tetrahydrofuran, acetonitrile or N,N-dimethylformamide, at 0-80° C. for 2-10 hours.

### Preparation of Aza-Cyclic 1H-Indole-2-carboxamides

Method A: An indole-2-carboxylic acid (1.0 mmol), an aza-cyclic amine (1.0 mmol) and N,N-diisopropylethylamine (3.0 mmol) were combined in tetrahydrofuran (l 0 mL). O-(7-azabenzotriazol-1-yl)-N,N,N′,N′-tetramethyluronium hexafluorophosphate (HATU) (Aldrich, 1.2 mmol) was then added, and the reaction mixture was stirred at ambient temperature for 6-10 hours. It was then quenched with water (5 mL) and extracted with ethyl acetate (3×20 mL). The combined extracts were washed with brine (2×10 mL) and dried over magnesium sulfate. The drying agents were removed by filtration. The organic solution was concentrated. The residue was then treated with an acid in an organic solvent (trifluoroacetic acid/dichloromethane/room temperature or p-toluenesulfonic acid/ethyl acetate/reflux) to remove t-butoxycarbonyl (Boc) protecting groups when present. The final product was purified by chromatography on silica gel or preparative HPLC [Waters, column: Nova-Pak® HR C18 6 μm 60 Å Prep-Pak® (25 mm×100 min), solvent: acetonitrile/water (v/v 0.1% trifluoroacetic acid), 5/95 to 95/5, flow rate of 40 mL/min. Fractions were collected based upon UV signal threshold and selected fractions were subsequently analyzed by flow injection analysis mass spectrometry using positive APCI ionization on a Finnigan LCQ using 70:30 methanol:10 mM aqueous ammonium hydroxide at a flow rate of 0.8 mL/min.; or Waters, column: Xbridge™ Prep C18 5□m, OBD™ 30×100 mm, solvent: acetonitrile/water (pH=10, prepared with ammonium bicarbonate/ammonium hydroxide) or acetonitrile/water (v/v 0.1% trifluoroacetic acid), 5/95 to 95/5, flow rate of 40 mL/min. Fractions were collected based upon UV signal threshold.]. In some cases, the free base of final product was stirred with an acid, such as hydrochloric acid or p-toluenesulfonic acid, in ethyl acetate at ambient temperature for 4-10 hours to give a salt.

Method B: To a solution of an indole-2-carboxylic acid (1.0 mmol) in N,N-dimethylformamide or tetrahydrofuran (anhydrous, 5 mL) was added N-(3-dimethylaminopropyl)-N′-ethylcarbodiimide hydrochloride (EDC) (Aldrich, 192 mg, 1.00 mmol) and 1-hydroxybenzotriazole (HOBt) hydrate (Fluka, 153 mg, 1.00 mmol). The mixture was stirred at ambient temperature for 10 minutes. An aza-cyclic amine (1.0 mmol) was added and the mixture was stirred at ambient temperature for 6-10 hours. It was then quenched with water (5 mL) and extracted with ethyl acetate (3×20 mL). The combined extracts were washed with brine (2×10 mL) and dried over magnesium sulfate. The drying agents were removed by filtration. The organic solution was concentrated. The residue was then treated with an acid in an organic solvent (trifluoroacetic acid/dichloromethane/room temperature or p-toluenesulfonic acid/ethyl acetate/reflux) to remove 1-butoxycarbonyl (Boc) protecting groups when present. The final product was purified by chromatography on silica gel or preparative HPLC [Waters, column: Nova-Pak® HR C18 6 μm 60 Å Prep-Pak® (25 mm×100 mm), solvent: acetonitrile/water (v/v 0.1% trifluoroacetic acid), 5/95 to 95/5, flow rate of 40 mL/min. Fractions were collected based upon UV signal threshold, and selected fractions were subsequently analyzed by flow injection analysis mass spectrometry using positive APCI ionization on a Finnigan LCQ using 70:30 methanol: 0 mM aqueous ammonium hydroxide at a flow rate of 0.8 mL/min.; or Waters, column: Xbridge™ Prep C18 5□m, OBD™ 30×100 mm, solvent: acetonitrile/water (pH=10 prepared with ammonium bicarbonate/ammonium hydroxide) or acetonitrile/water (v/v 0.1% trifluoroacetic acid), 5/95 to 95/5, flow rate of 40 ml/min. Fractions were collected based upon UV signal threshold.]. In some cases, the free base of final product was stirred with an acid, such as hydrochloric acid or p-toluenesulfonic acid, in ethyl acetate at ambient temperature for 4-10 hours to give a salt.

### Preparation of N-Methyl Aza-Cyclic 1H-Indole-2-carboxamide

Method C: A solution of aza-cyclic 1H-indole-2-carboxamide (0.5 mmol) prepared by either Method A or B and formaldehyde (Aldrich, aqueous, 37%, 1.0-1.5 mmol) in acetonitrile (5 mL) was stirred with sodium triacetoxyborohydride (Aldrich, 1.0 mmol) at ambient temperature for 4-10 hours. It was then quenched with water (5 mL) and extracted with chloroform (3×20 mL). The combined extracts were washed with brine (2×10 mL) and concentrated. The residue was then was purified by chromatography on silica gel (dichloromethane/methanol/ammonium hydroxide=90/10/2) or preparative HPLC [Waters, column: Xbridge™ Prep C18 5 μm, OBD™ 30×100 mm, solvent: acetonitrile/water (pH=10, prepared with ammonium bicarbonate/ammonium hydroxide) or acetonitrile/water (v/v 0.1% trifluoroacetic, acid), 5/95 to 95/5, flow rate of 40 mL/min. Fractions were collected based upon UV signal threshold.]. In some cases, the free base of final product was stirred with an acid, such as hydrochloric acid or p-toluenesulfonic acid, in ethyl acetate at ambient temperature for 4-10 hours to give a salt.

### Example 1

### (R)-3,4,7-trimethyl-N-(pyrrolidin-3-yl)-1H-indole-2-carboxamide

The title compound was prepared according to Method A using (R)-tert-butyl 3-aminopyrrolidine-1-carboxylate (Aldrich) and 3,4,6-trimethyl-1H-indole-2-carboxylic acid (Enamine). ¹H NMR (300 MHz, DMSO-d₆) δ 1.56-1.74 (m, 1H), 1.92-2.12 (m, 1H), 2.42 (s, 3H), 2.61 (s, 3H), 2.64-2.69 (m, 1H), 2.71 (s, 3H), 2.73-2.84 (m, 1H), 2.85-2.97 (m, 1H), 3.02 (dd, J=11.3, 6.5 Hz, 1H), 4.26-4.56 (m, 1H), 6.63 (d, J=7.1 Hz, 1H), 6.82 (d, J=7.5 Hz, 1H), 8.02 (d, J=7.1 Hz, 1H), 10.62 (s, 1H) ppm; MS (DCI/NH₃) m/z 272 (M+H)⁺.

### Example 2

### (S)-3,4,7-trimethyl-N-(pyrrolidin-3-yl)-1H-indole-2-carboxamide

The title compound was prepared according to Method A using (S)-tert-butyl 3-aminopyrrolidine-1-carboxylate (Aldrich) and 3,4,6-trimethyl-1H-indole-2-carboxylic acid (Enamine). ¹H NMR (300 MHz, DMSO-d₆) δ 1.59-1.78 (m, 1H), 1.92-2.13 (m, 1H), 2.42 (s, 3H), 2.61 (s, 3H), 2.65-2.73 (m, 1H), 2.71 (s, 3H), 2.75-2.86 (m, 1H), 2.88-3.00 (m, 1H), 3.04 (dd, J=11.2, 6.4 Hz, 1H), 4.23-4.43 (m, 1H), 6.63 (d, J=7.5 Hz, 1H), 6.82 (d, J=7.8 Hz, 1H), 8.05 (d, J=6.8 Hz, 1H), 10.63 (s, 1H) ppm; MS (DCI/NH₃) m/z 272 (M+H)⁺.

### Example 3

### (S)-4,6-dichloro-N-(pyrrolidin-3-yl)-1H-indole-2-carboxamide trifluoroacetate

The title compound was prepared according to Method A using (S)-tert-butyl 3-aminopyrrolidine-1-carboxylate (Aldrich) and 4,6-dichloro-1H-indole-2-carboxylic acid (Astatech). ¹H NMR (300 MHz, MeOH-d₄) δ 2.11-2.29 (m, 1H), 2.33-2.57 (m, 1H), 3.35-3.47 (m, 2H), 3.50-3.66 (m, 2H), 4.50-4.67 (m, 1H), 7.14 (d, J=1.7 Hz, 1H), 7.24 (d, J=1.10 Hz, 1H), 7.43 (dd, J=1.7, 1.0 Hz, 1H) ppm; MS (DCI/NH₃) m/z 298, 300 (M+H)⁺.

### Example 4

### (R)-4,6-dimethyl-N-(pyrrolidin-3-yl)-1H-indole-2-carboxamide

The title compound was prepared according to Method A using (R)-tert-butyl 3-aminopyrrolidine-1-carboxylate (Aldrich) and 4,6-dimethyl-1H-indole-2-carboxylic acid (Enamine). ¹H NMR (300 MHz, DMSO-d₆) δ 1.56-1.75 (m, 1H), 1.90-2.07 (m, 1H), 2.34 (s, 3H), 2.43 (s, 3H), 2.65 (dd, J=11.2, 4.7 Hz, 1H), 2.70-2.83 (m, 1H), 2.85-3.03 (m, 2H), 4.18-4.48 (m, 1H), 6.66 (s, 1H), 7.02 (s, 1H), 7.13 (s, 1H), 8.23 (d, J=7.5 Hz, 1H), 11.32 (s, 1H) ppm; MS (DCI/NH₃) m/z 258 (M+H)⁺.

### Example 5

### (R)-4,6-dimethyl-N-(piperidin-3-yl)-1H-indole-2-carboxamide

The title compound was prepared according to Method B using (R)-tert-butyl 3-aminopiperidine-1-carboxylate (Fluka) and 4,6-dimethyl-1H-indole-2-carboxylic acid (Enamine). ¹H NMR (300 MHz, DMSO-d₆) δ 1.32-1.55 (m, 2H), 1.57-1.69 (m, 1H), 1.80-1.93 (m, 1H), 2.08-2.26 (m, 1H), 2.33 (s, 3H), 2.35-2.41 (m, 1H), 2.43 (s, 3H), 2.79 (d, J=12.3 Hz, 1H), 2.97 (dd, J=11.9, 3.2 Hz, 1H), 3.63-3.93 (m, 1H), 6.66 (s, 1H), 7.01 (s, 1H), 7.14 (d. J=1.2 Hz, 1H), 8.04 (d, J=8.3 Hz, 1H), 11.33 (s, 1H) ppm; MS (DCI/NH₃) m/z 272 (M+H)⁺.

### Example 6

### (S)-3,4,7-trimethyl-N-(piperidin-3-yl)-1H-indole-2-carboxamide

The title compound was prepared according to Method B using (S)-tert-butyl 3-aminopiperidine-1-carboxylate (Fluka) and 3,4,7-dimethyl-1H-indole-2-carboxylic acid (Oakwood). ¹H NMR (300 MHz, DMSO-d₆) δ 1.37-1.48 (m, 2H), 1.57-1.75 (m, 1H), 1.80-1.96 (m, 1H), 2.08-2.30 (m, 1H), 2.41-2.45 (m, 1H), 2.42 (s, 3H), 2.61 (s, 3H), 2.71 (s, 3H), 2.74-2.87 (m, 1H), 3.02 (dd, J=11.1, 3.2 Hz, 1H), 3.70-3.97 (m, 1H), 6.63 (d, J=7.9 Hz, 1H), 6.82 (d, J=7.1 Hz, 1H), 7.79 (d, J=7.9 Hz, 1H), 10.63 (s, 1H) ppm; MS (DCI/NH₃) m/z 286 (M+H)⁺.

### Example 7

### (R)-4,6-dimethyl-N-(1-methylpyrrolidin-3-1)-1H-indole-2-carboxamide

The title compound was prepared according to Method C using the product of Example 4. ¹H NMR (300 MHz, DMSO-d₆) δ 1.61-1.85 (m, 1H), 2.08-2.24 (m, 1H), 2.26 (s, 3H), 2.33 (s, 3H), 2.34-2.43 (m, 2H), 2.44 (s, 3H), 2.54-2.76 (m, 2H), 4.30-4.52 (m, 1H), 6.66 (s, 1H), 7.01 (s, 1H), 7.19 (s, 1H), 8.37 (d, J=7.1 Hz, 1H), 11.31 (s, 1H) ppm; MS (DCI/NH₃) m/z 272 (M+H)⁺.

### Example 8

### (R)-4,6-dimethyl-N-(1-methylpiperidin-3-yl)-1H-indole-2-carboxamide

The title compound was prepared according to Method C using the product of Example 5. ¹H NMR (300 MHz, DMSO-d₆) δ 1.33 (td, J=11.5, 4.0 Hz, 1H), 1.45-1.60 (m, 1H), 1.63-1.93 (m, 4H), 2.18 (s, 3H), 2.33 (s, 3H), 2.43 (s, 3H), 2.63 (d, J=10.7 Hz, 1H), 2.79 (dd, J=10.5, 3.8 Hz, 1H), 3.75-4.14 (it, 1H), 6.66 (s, 1H), 7.01 (s, 1H), 7.15 (s, 1H), 8.08 (d, J=7.9 Hz, 1H), 11.34 (s, 1H) ppm; MS (DCI/NH₃) m/z 286 (M+H)⁺.

### Example 9

### (S)-3,4,7-trimethyl-N-(1-methylpiperidin-3-yl)-1H-indole-2-carboxamide

The title compound was prepared according to Method C using the product of Example 6. ¹H NMR (300 MHz, DMSO-d₆) δ 1.21-1.41 (m, 1H), 1.44-1.63 (m, 1H), 1.64-2.04 (m, 4H), 2.19 (s, 3H), 2.42 (s, 3H), 2.55-2.63 (m, 1H), 2.61 (s, 3H), 2.71 (s, 3 H), 2.81 (dd, J=10.1, 3.4 Hz, 1H), 3.74-4.55 (m, 1H), 6.63 (d, J=7.5 Hz, 1H), 6.83 (d, J=7.1 Hz, 1H), 7.85 (d, J=7.5 Hz, 1H), 10.65 (s, 1H) ppm; MS (DCI/NH₃) m/z 300 (M+H)⁺.

### Example 10

### (R)-6-tert-butyl-N-(pyrrolidin-3-yl)-1H-indole-2-carboxamide

The title compound was prepared according to Method A using (R)-tert-butyl 3-aminopyrrolidine-1-carboxylate (Aldrich) and 6-tert-butyl-1H-indole-2-carboxylic acid (Enamine). ¹H NMR (300 MHz, MeOH-d₄) δ 1.37 (s, 9H), 1.78-1.94 (m, 1H), 2.13-2.30 (m, 1H), 2.87 (dd, J=11.9, 4.7 Hz, 1H), 2.91-3.03 (m, 1H), 3.06-3.16 (m, 1H), 3.20 (dd, J=11.9, 6.8 Hz, 1H), 4.36-4.58 (m, 1H), 7.04 (d, J=0.7 Hz, 1H), 7.18 (dd, J=8.6, 1.9 Hz, 1H), 7.42-7.44 (m, 1H), 7.51 (d, J=9.2 Hz, 1H) ppm; MS (DCI/NH₁₃) m/z 286 (M+H)⁺.

### Example 11

### (R)-6-tert-butyl-N-(piperidin-3-yl)-1H-indole-2-carboxamide

The title compound was prepared according to Method A using (R)-tert-butyl 3-aminopiperidine-1-carboxylate (Fluka) and 6-tert-butyl-1H-indole-2-carboxylic acid (Enamine). ¹H NMR (300 MHz, MeOH-d₄) δ 1.37 (s, 9H), 1.55-1.67 (m, 2H), 1.72-1.82 (m, 1H), 1.93-2.16 (m, 1H), 2.50-2.64 (m, 2H), 2.85-2.99 (m, 1H), 3.15 (dd, J=12.1, 4.6 Hz, 1H), 3.86-4.12 (m, 1H), 7.05 (d, J=0.8 Hz, 1H), 7.18 (dd, J=8.3, 1.6 Hz, 1H), 7.43 (s, 1H), 7.51 (d, J=8.3 Hz, 1H) ppm; MS (DCI/NH₃) m/z 300 (M+H)⁺.

### Example 12

### (R)-6-phenyl-N-(piperidin-3-yl)-1H-indole-2-carboxamide

The title compound was prepared according to Method B using (R)-tert-butyl 3-aminopiperidine-1 carboxylate (Fluka) and 6-phenyl-1H-indole-2-carboxylic acid (Enamine). ¹H NMR (300 MHz, MeOH-d₄) δ 1.54-1.71 (m, 2H), 1.75-1.89 (m, 1H), 1.98-2.12 (m, 1H), 2.54-2.69 (m, 2H), 2.90-3.01 (m, 1H), 3.19 (dd, J=12.2, 3.7 Hz, 1H), 3.89-4.20 (m, 1H), 7.13 (d, J=0.7 Hz, 1H), 7.26-7.49 (m, 4H), 7.61-7.71 (m, 4H) ppm; MS (DCI/NH₃) m/z 320 (M+H)⁺.

### Example 13

### (R)-6-phenyl-N-(pyrrolidin-3-yl)-1H-indole-2-carboxamide

The title compound was prepared according to Method B using (R)-tert-butyl 3-aminopyrrolidine-1-carboxylate (Aldrich) and 6-phenyl-1H-indole-2-carboxylic acid (Enamine). 1H NMR (300 MHz, MeOH-d₄) δ 1.78-1.96 (m, 1H), 2.13-2.32 (m, 1H), 2.88 (dd, J=11.9, 4.7 Hz, 1H), 2.94-3.04 (m, 1H), 3.06-3.16 (m, 1H), 3.21 (dd, J=11.5, 6.8 Hz, 1H), 4.38-4.59 (m, 1H), 7.12 (d, J=1.0 Hz, 1H), 7.25-7.48 (m, 4H), 7.59-7.70 (m, 4H) ppm; MS (DCI/NH₃) m/z 306 (M+H)⁺.

### Example 14

### (R)-4,5-dimethyl-N-(pyrrolidin-3-yl)-1H-indole-2-carboxamide

### Example 14A

### (Z)-methyl 2-azido-3-(2,3-dimethylphenyl)acrylate

At −20° C., to a solution of sodium methoxide (Aldrich, 0.5 M, 40.0 mL, 20.00 mmol) in methanol, was slowly added a solution of 2,3-dimethylbenzaldehyde (Aldrich, 2.147 g, 16 mmol) and ethyl azidoacetate (Fluka, 25% in toluene, 25.0 g, 48.4 mmol) in tetrahydrofuran (20 mL) to maintain the reaction temperature less than −5° C. After the completion of the addition, the reaction mixture was stirred at −10° C. for 2 hours. Then the reaction was quenched with water (30 mL) and extracted with ether (3×50 mL). The combined extracts were washed with brine (2×20 mL), concentrated, and purified with chromatography on silica (hexanes/ethyl acetate, v/v 4/1, R₁=0.7) to give the title compound. ¹H NMR (300 MHz, CDCl₃) δ 2.24 (s, 3H), 2.30 (s, 3H), 3.92 (s, 3H), 7.09-7.18 (m, 2H), 7.20 (s, 1H), 7.55-7.69 (m, 1H) ppm; MS (DCI/NH₃) m/z 249 (M+NH₄)⁺.

### Example 14B

### methyl 4,5-dimethyl-1H-indole-2-carboxylate

The solution of the product of Example 14A (2.44 g, 10.6 mmol) in toluene (20 mL) was degassed and purged with nitrogen for three times. Rhodium(II) heptafluorobutyrate dimer (Aldrich, 0.21 g, 0.2 mmol) was added under nitrogen. The slightly brown solution was then stirred at 80° C. for 15 hours, cooled to ambient temperature, concentrated under reduced pressure and the residue was purified with chromatography on silica gel (hexane/ethyl acetate, v/v 9/1, R₁ 0.6) to give the title compound. ¹H NMR (300 MHz, DMSO-d₆) δ 2.28 (s, 3H), 2.39 (s, 3H), 3.86 (s, 3H), 7.06 (d, J=7.5 HZ, 1H), 7.12-7.25 (m, 2H) ppm; MS (DCI/NH₃) m/z 204 (M+H)⁺.

### Example 14C

### 4,5-dimethyl-1H-indole-2-carboxylic acid

The solution of the product of Example 14B (1.60 g, 7.4 mmol) in ethanol (50 mL) was stirred with potassium hydroxide (Aldrich, 0.83 g, 14.8 mmol) at reflux for 15 hours. It was then cooled to ambient temperature, acidified with concentrated hydrochloric acid to pH=1 at 15-25° C. The mixture was extracted with ethyl acetate (3×50 mL). The combined extracts were washed with brine (2×10 mL) and dried over magnesium sulfate. The drying agent was removed by filtration. The organic solution was concentrated under reduced pressure to supply the title compound. ¹H NMR (300 MHz, DMSO-d₆) δ 2.28 (s, 3H), 2.38 (s, 3H), 7.03 (d, J=8.4 Hz, 1H), 7.10 (dd, J=2.2, 0.8 Hz, 1H), 7.14 (d, J=8.2 Hz, 1H), 11.57 (s, 1H), 12.82 (s, 1H) ppm; MS (DCI/NH₃) m/z 190 (M+H)⁺.

### Example 14D

### (R)-4,5-dimethyl-N-(pyrrolidin-3-yl)-1H-indole-2-carboxamide

The title compound was prepared according to Method A using (R)-tert-butyl 3-aminopyrrolidine-1-carboxylate (Aldrich) and the product of Example 14C. ¹H NMR (300 MHz, MeOH-d₄) δ 1.71-1.95 (m, 1H), 2.07-2.27 (m, 1H), 2.33 (s, 3H), 2.44 (s, 3H), 2.83 (dd, J=11.9, 4.8 Hz, 1H), 2.87-2.99 (m, 1H), 3.02-3.13 (m, 1H), 3.16 (dd, J=11.9, 6.7 Hz, 1H), 4.37-4.60 (m, 1H), 6.99-7.05 (m, 1H), 7.12-7.19 (m, 2H) ppm; MS (DCI/NH₃) m/z 258 (M+H)⁺.

### Example 15

### (R)-4,5-dimethyl-N-(piperidin-3-yl)-1H-indole-2-carboxamide

The title compound was prepared according to Method A using (R)-tert-butyl 3-aminopiperidine-1-carboxylate (Fluka) and the product of Example 14C. ¹H NMR (300 MHz, MeOH-d₄) δ 1.53-1.67 (m, 2H), 1.71-1.86 (m, 1H), 1.93-2.08 (m, 1H), 2.33 (s, 3H), 2.45 (s, 3H), 2.50-2.66 (m, 2H), 2.84-2.98 (m, 1H), 3.15 (dd, J=12.3, 4.0 Hz, 1H), 3.86-4.15 (m, 1H), 7.02 (d, J=8.4 Hz, 1H), 7.10-7.26 (m, 2H) ppm; MS (DCI/NH₃) m/z 272 (M+H)⁺.

### Example 16

### N-(azepan-3-yl)-4,6-dichloro-1H-indole-2-carboxamide

The title compound was prepared according to Method A using azepan-3-amine (Astatech) and 4,6-dichloro-1H-indole-2-carboxylic acid (Astatech). ¹H NMR (300 MHz, MeOH-d₄) δ 1.42-1.81 (m, 5. H), 1.82-1.95 (m, 1H), 2.74 (dd, J=13.7, 8.0 Hz, 1H), 2.85 (t, J=5.6 Hz, 2H), 3.05 (dd, J=13.6, 4.4 Hz, 1H), 3.84-4.36 (m, 1H), 7.23 (d, J=1.4 Hz, 1H), 7.33 (d, J=0.7 Hz, 1H), 7.40-7.43 (m, 1H), 8.48 (d, J=8.1 Hz, 1H) ppm; MS (DCI/NH₃) m/z 326 (M+H)⁺, 328 (M+H)⁺.

### Example 17

### N-(azepan-3-yl)-3,4,7-trimethyl-1H-indole-2-carboxamide

The title compound was prepared according to Method A using azepan-3-amine (Astatech) and 3,4,7-trimethyl 1H-indole-2-carboxylic acid (Oakwood). ¹H NMR (300 MHz, DMSO-d₆) δ 1.38-1.80 (m, 5H), 1.81-1.97 (m, 1H), 2.42 (s, 3H), 2.61 (s; 3H), 2.71 (s, 3H), 2.71-2.89 (m, 3H), 3.00 (dd, J=13.7, 4.6 Hz, 1H), 3.94-4.15 (m, 1H), 6.63 (d, J=7.1 Hz, 1H), 6.82 (d, J-7.1 Hz, 1H), 7.86 (d, J=7.9 Hz, 1H), 10.67 (s, 1H) ppm; MS (DCI/NH₃) m/z 300 (M+H)⁺.

### Example 18

### (R)-6-methoxy-N-(piperidin-3-yl)-1H-indole-2-carboxamide

The title compound was prepared according to Method A using (R)-tert-butyl 3-aminopiperidine-1-carboxylate (Fluka) and 6-methoxy-1H-indole-2-carboxylic acid (Enamine). ¹H NMR (300 MHz, MeOH-d₄) δ 1.50-1.68 (m, 2H), 1.69-1.86 (m, 1H), 1.95-2.15 (m, 1H), 2.47-2.65 (m, 2H), 2.85-3.02 (m, 1H), 3.15 (dd, J=12.2, 3.4 Hz, 1H), 3.82 (s, 3H), 3.90-4.15 (m, 1H), 6.72 (dd, J=8.6, 2.2 Hz, 1H), 6.92 (d, J=2.4 Hz, 1H), 7.04 (d, J=1.0 Hz, 1H), 7.45 (d, J=8.8 Hz, 1H) ppm; MS (DCI/N₃) m/z 274 (M+H)⁺.

### Example 19

### (R)-6-methoxy-N-(pyrrolidin-3-yl)-1H-indole-2-carboxamide

The title compound was prepared according to Method A using (R)-tert-1-butyl 3-aminopyrrolidine-1-carboxylate, (Aldrich) and 6-methoxy-1H-indole-2-carboxylic acid (Enamine). ¹H NMR (300 MHz, MeOH-d₄) δ 1.76-1.98 (m, 1H), 2.15-2.31 (m, 1H), 2.87 (dd, J=11.7, 4.9 Hz, 1H), 2.91-3.03 (m, 1H), 3.04-3.16 (m, 1H), 3.20 (dd, J=11.7, 7.0 Hz, 1H), 3.82 (s, 3H), 4.39-4.57 (m, 1H), 6.72 (dd, J=8.6, 2.2 Hz, 1H), 6.92 (d, J=2.4 Hz, 1H), 7.03 (s, 1H), 7.45 (d, J=8.8 Hz, 1H) ppm; MS (DCI/NH₃) m/z 260 (M+H)⁺.

### Example 20

### (R)-6-chloro-7-methyl-N-(pyrrolidin-3-yl)-1H-indole-2-carboxamide

The title compound was prepared according to Method A using (R)-tert-butyl 3-aminopyrrolidine-1-carboxylate (Aldrich) and 6-chloro-7-methyl-1H-indole-2-carboxylic acid (Matrix Scientific). ¹H NMR (300 MHz, MeOH-d₄) δ 1.75-1.94 (m, 1H), 2.12-2.30 (m, 1H), 2.86 (dd, J=11.7, 5.0 Hz, 1H), 2.91-3.02 (m, 1H), 3.04-3.16 (m, 1H), 3.20 (dd, J=11.5, 6.7 Hz, 1H), 4.37-4.62 (m, 1H), 7.07 (d, J=8.7 Hz, 1H), 7.10 (s, 1H), 7.41 (d, J=8.7 Hz, 1H) ppm; MS (DCI/NH₃) m/z 278 (M+H)⁺, 280 (M+H)⁺.

### Example 21

### (R)-6-chloro-7-methyl-N-(piperidin-3-yl)-1H-indole-2-carboxamide

The title compound was prepared according to Method A using (R)-tert-butyl 3-aminopiperidine-1-carboxylate (Fluka) and 6-chloro-7-methyl-1H-indole-2-carboxylic acid (Matrix Scientific). ¹H NMR (300 MHz, MeOH-d₄) δ 1.51-1.68 (m, 2H), 1.72-1.88 (m, 1H), 1.95-2.08 (m, 1H), 2.50-2.67 (m, 5H), 2.87-3.01 (m, 1H), 3.17 (dd, J=12.2, 3.4 Hz, 1H), 3.92-4.08 (m, 1H), 7.06 (d, J=8.5 Hz, 1H), 7.11 (s, 1H), 7.40 (d, J=8.5 Hz, 1H) ppm; MS (DCI/NH₃) m/z 292 (M+H)⁺, 294 (M+H)⁺.

### Example 22

### (S)-6-chloro-7-methyl-N-(pyrrolidin-3-yl)-1H-indole-2-carboxamide

The title compound was prepared according to Method A using (S)-tert-butyl 3-aminopyrrolidine-1-carboxylate (Aldrich) and 6-chloro-7-methyl-1H-indole-2-carboxylic acid (Matrix Scientific). ¹H NMR (300 MHz, MeOH-d₄) δ 1.76-1.94 (m, 1H), 2.10-2.32 (m, 1H), 2.56 (s, 3H), 2.85 (dd, J=11.9, 4.7 Hz, 1H), 2.90-3.02 (m, 1H), 3.03-3.15 (m, 1H), 3.20 (dd, J=11.9, 6.8 Hz, 1H), 4.40-4.58 (m, 1H), 7.07 (d, J=8.8 Hz, 1H), 7.10 (s, 1H), 7.41 (d, J=9.2 Hz, 1H) ppm; MS (DCI/NH₃) m/z 278 (M+H)⁺, 280 (M+H)⁺.

### Example 23

### (S)-6-chloro-7-methyl-N-(piperidin-3-yl)-1H-indole-2-carboxamide

The title compound was prepared according to Method A using (S)-tert-butyl 3-aminopiperidine-1-carboxylate (Fluka) and 6-chloro-7-methyl-1H-indole-2-carboxylic acid (Matrix Scientific). ¹H NMR (300 MHz, MeOH-d₄) δ 1.50-1.69 (m, 2H), 1.70-1.86 (m, 1H), 1.95-2.12 (m, 1H), 2.50-2.73 (m, 5H), 2.87-3.00 (m, 1H), 3.17 (dd. J=12.1, 3.8. Hz, 1H), 3.93-4.21 (m, 1H), 7.07 (d, J=8.3 Hz, 1H), 7.11 (s, 1H), 7.41 (d, J=8.3 Hz, 1H) ppm; MS (DCI/NH₃) m/z 292 (M+H)⁺, 294 (M+H)⁺.

### Example 24

### (R)-5-bromo-N-(pyrrolidin-3-yl)-1H-indole-2-carboxamide

The title compound was prepared according, to Method B using (R)-tert-butyl 3-aminopyrrolidine-1-carboxylate (Aldrich) and 5-bromo-1H-indole-2-carboxylic acid (Aldrich). ¹H NMR (300 MHz, MeOH-d₄) δ 1.88-2.03 (m, 1H), 2.15-2.33 (m, 1H), 2.95-3.47 (m, 4H), 4.48-4.59 (m, 1H), 7.04-7.17 (m, 1H), 7.24-7.41 (m, 2H), 7.75 (d, J=2.0 Hz, 1H) ppm; MS (DCI/NH₃) m/z 308 (M+H)⁺, 310 (M+H)⁺.

### Example 25

### (R)-1-methyl-(pyrrolidin-3-yl)-1H-indole-2-carboxamide

The title compound was prepared according to Method B using (R)-tert-butyl 3-aminopyrrolidine-1-carboxylate (Aldrich) and 1-methyl-1H-indole-2-carboxylic acid (Aldrich). ¹H NMR (300 MHz, MeOH-d₄) δ 1.79-1.92 (m, 1H), 2.14-2.29 (m, 1H), 2.88 (dd, J=11.90, 4.76 Hz, 1H), 2.92-3.02 (m, 1H), 3.05-3.16 (m, 1H), 3.21 (dd, J=11.90, 6.74 Hz, 1H), 3.99 (s, 3H), 4.43-4.53 (m, 1H), 7.02 (s, 1H), 7.09 (ddd, J=8.0, 7.0, 1.2 Hz, 1H), 7.28 (ddd, J=8.3, 7.1, 1.2 Hz, 1H), 7.44 (dd, J=8.3, 0.8 Hz, 1H) ppm; MS (DCI/NH₃) m/z 244 (M+H)⁺.

### Example 26

### (R)-5-bromo-N-(1-methylpyrrolidin-3-yl)-1H-indole-2-carboxamide

The title compound was prepared according to Method C using the product of Example 24. ¹H NMR (300 MHz, MeOH-d₄) δ 1.80-1.94 (m, 1H), 2.30-2.44 (m, 4H), 2.52-2.66 (m, 2H), 2.82 (td, J=9.0, 5.8 Hz, 1H), 2.90 (dd, J=9.9, 7.1 Hz, 1H), 4.53-4.63 (m, 1H), 7.05 (s, 1H), 7.27-7.32 (m, 1H), 7.34-7.39 (m, 1H), 7.75 (d, J=2.0 Hz, 1H) ppm; MS (DCI/NH₃) m/z 322 (M+H)⁺, 324 (M+H)⁺.

### Example 27

### (R)-4,6-dichloro-N-(pyrrolidin-3-yl)-1H-indole-2-carboxamide

The title compound was prepared according to Method B using (R)-tert-butyl 3-aminopyrrolidine-1-carboxylate (Aldrich) and 4,6-dichloro-1H-indole-2-carboxylic acid (Astatech). ¹H NMR (300 MHz, MeOH-d₄) δ 1.85-1.98 (m, 1H), 2.17-2.30 (m, 1H), 2.92-2.99 (m, 1H), 3.00-3.08 (m, 1H), 3.12-3.20 (m, 1H), 3.25 (dd, J=11.9, 6.8 Hz, 1H), 4.46-4.57 (m, 1H), 7.12 (d, J=1.7 Hz, 1H), 7.23 (d, J=1.0 Hz, 1H), 7.42 (dd, J=1.7, 1.0 Hz, 1H) ppm; MS (DCI/NH₃) m/z 298 (M+H)⁺, 300 (M+H)⁺.

### Example 28

### (A)-4-methoxy-N-(pyrrolidin-3-yl)-1H-indole-2-carboxamide

The title compound was prepared according to Method B using (R)-tert-butyl 3-aminopyrrolidine-1-carboxylate (Aldrich) and 4-methoxy-1H-indole-2-carboxylic acid (Oakwood). ¹H NMR (300 MHz, MeOH-d₄) δ 1.87-1.99 (m, 1H), 2.18-2.31 (m, 1H), 2.93-3.10 (m, 2H), 3.14-3.28 (m, 2H), 3.92 (S, 3H), 4.46-4.56 (m, 1H), 6.51 (d, J=7.9 Hz, 1H), 6.99-7.05 (m, 1H), 7.14 (t, J=8.1 Hz, 1H), 7.20 (s, 1H) ppm; MS (DCI/NH₃) m/z 260 (M+H)⁺.

### Example 29

### (R)-5-chloro-N-(pyrrolidin-3-yl)-1H-indole-2-carboxamide

The title compound was prepared according to Method B using (R)-tert-butyl 3-aminopyrrolidine-1-carboxylate (Aldrich) and 5-chloro-1H-indole-2-carboxylic acid (Aldrich). ¹H NMR (300 MHz, MeOH-d₄) δ 1.83-1.95 (m, 1H), 2.15-2.30 (m, 1H), 2.92 (dd, J=11.9, 4.8 Hz, 1H), 3.00 (ddd, J=11.4, 8.1, 6.3 Hz, 1H), 3.15 (ddd, J=11.3, 8.1, 6.4 Hz, 1H), 3.23 (dd, J=11.9, 6.8 Hz, 1H), 4.45-4.56, (m, 1H), 7.06 (d, J=1.0 Hz, 1H), 7.17 (dd, J=8.8, 2.0 Hz, 1H), 7.41 (d, J=8.8 Hz, 1H), 7.59 (d, J=2.0 Hz, 1H) ppm; MS (DCI/NH₃) m/z 264 (M+H)⁺, 266 (M+H)⁺.

### Example 30

### (R)-4,6-dichloro-N-(1-methylpyrrolidin-3-yl)-1H-indole-2-carboxamide

The title compound was prepared according to Method C using the product of Example 27. ¹H NMR (300 MHz, MeOH-d₄) δ 1.81-1.94 (m, 1H), 2.30-2.43 (m, 4H), 2.52-2.66 (m, 2H), 2.82 (td, J=8.9, 5.9 Hz, 1H), 2.90 (dd, J=10.2, 7.1 Hz, 1H), 4.53-4.63 (m, 1H), 7.12 (d, J=1.7 Hz, 1H), 7.22 (d, J=0.7 Hz, 1H), 7.42 (dd, J=1.7, 1.0 Hz, 1H) ppm; MS (DCI/NH₃) m/z 312 (M+H)⁺, 314 (M+H)⁺.

### Example 31

### (R)-5-chloro-N-1-methylpyrrolidin-3-yl)-1H-indole-2-carboxamide

The title compound was prepared according to Method C using the product of Example 29. ¹H NMR (300 MHz, MeOH-d₄) δ 1.80-1.93 (m, 1H), 2.30-2.44 (m, 4H), 2.52-2.66 (m, 2H), 2.81 (td, J=9.0, 5.8 Hz, 1H), 2.89 (dd, J=10.2, 7.1 Hz, 1H), 4.52-4.64 (m, 1H), 7.05 (s, 1H), 7.17 (dd, J=8.8, 2.0 Hz, 1H), 7.40 (d, J=8.8 Hz, 1H), 7.59 (d, J=2.0 Hz, 1H) ppm; MS (DCI/NH₃) m/z 278 (M+H)⁺, 280 (M+H)⁺.

### Example 32

### (R)-4-methoxy-N-(1-methylpyrrolidin-3-yl)-1H-indole-2-carboxamide

The title compound was prepared according to Method C using the product of Example 28. ¹H NMR (300 MHz, MeOH-d₄) δ 1.80-1.93 (m, 1H), 2.30-2.44 (m, 4H), 2.53-2.67 (m, 2H), 2.77-2.85 (m, 1H), 2.89 (dd, J=10.1, 7.3 Hz, 1H), 3.92 (s, 3H), 4.52-4.63 (m, 1H), 6.50 (d, J=7.5 Hz, 1H), 7.02 (d, J=8.3 Hz, 1H), 7.14 (t, J=7.5 Hz, 1H), 7.19 (d, J=0.8 Hz, 1H) ppm; MS (DCI/NH₃) m/Z 274 (M+H)⁺.

### Example 33

### (R)-N,3,4,7-tetramethyl-N-(pyrrolidin-3-yl)-1H-indole-2-carboxamide

### Example 33A

### (R)-N-(1-benzylpyrrolidin-3-yl)-N,3,4,7-tetramethyl-1H-indole-2-carboxamide

The title compound was prepared according to Method B using (R)-1-benzyl-N-methylpyrrolidin-3-amine (VWR) and 3,4,7-trimethyl-1H-indole-2-carboxylic acid (Chembridge). ¹H NMR (300 MHz, MeOH-d₄) δ 1.89-2.07 (m, 1H), 2.07-2.28 (m, 1H), 2.39-2.45 (m, 1H), 2.43 (s, 3H), 2.56-2.63 (m, 1H), 2.63 (s, 3H), 2.73-2.78 (m, 1H), 2.80-2.91 (m, 1H), 3.08 (s, 3H), 3.43-3.84 (m, 3H), 6.65 (d, J=7.1 Hz, 1H), 6.79 (d, J=7.1 Hz, 1H), 7.16-7.45 (m, 5H) ppm; MS (DCI/NH₃) m/z 376 (M+H)⁺.

### Example 33B

### (R)-N,3,4,7-tetramethyl-N-(pyrrolidin-3-yl)-1H-indole-2-carboxamide

A solution of the product of Example 33A (120 mg, 0.32 mmol) in methanol (5 mL) was stirred with palladium on carbon (Aldrich, 10 weight %, 40 mg) under hydrogen at ambient temperature for 10 hours. The catalyst was then removed by a careful filtration under nitrogen. The filtrate was concentrated to give the title compound. ¹H NMR (300 MHz, MeOH-d₄) δ 1.91-2.21 (m, 2H); 2.40 (s, 3H), 2.47 (s, 3H), 2.64 (s, 3H), 2.83-3.22 (m, 7H), 4.60-4.74 (m, 1H), 6.66 (d, J=7.5 Hz, 1H), 6.81 (d, J=7.1 Hz, 1H) ppm; MS (DCI/NH₃) m/z 286 (M−H)⁺.

### Example 34

### (R)-N,3,4,7-tetramethyl-N-(1-methylpyrrolidin-3-yl)-1H-indole-2-carboxamide

The title compound was prepared according to Method C using the product of Example 33B. ¹H NMR (300 MHz, MeOH-d₄) δ 1.89-2.09 (m, 1H), 2.08-2.26 (m, 1H), 2.35 (s, 3H), 2.39 (s, 3H), 2.42-2.55 (m, 4H), 2.64 (s, 3H), 2.67-2.82 (m, 3H), 3.06 (s, 3H), 4.67-4.92 (m, 1H), 6.66 (d, J=7.1 Hz, 1H), 6.80 (d, J=7.1 Hz, 1H) ppm; MS (DCI/NH₃) m/z 300 (M+H)⁺.

### Example 35

### (S)-5-fluoro-N-(pyrrolidin-3-yl)-1H-indole-2-carboxamide

The title compound was prepared according to Method A using (S)-tert-butyl 3-aminopyrrolidine-1-carboxylate (Aldrich) and 5-fluoro-1H-indole-2-carboxylic acid (Aldrich). ¹H NMR (300 MHz, MeOH-d₄) δ 1.78-1.96 (m, 1H), 2.12-2.31 (m, 1H), 2.88 (dd, J=11.9, 4.8 Hz, 1H), 2.92-3.03 (m, 1H), 3.04-3.16 (m, 1H), 3.21 (dd, J=11.9, 6.7 Hz, 1H), 4.34-4.57 (m, 1H), 7.00 (td, J=9.2, 2.6 Hz, 1H), 7.07 (s, 1H), 7.26 (dd, J=9.5, 2.8 Hz, 1H), 7.41 (dd, J=9.1, 4.4 Hz, 1H) ppm; MS (DCI/NH₃) m/z 248 (M+H)⁺.

### Example 36

### (S)-5-methoxy-N-(pyrrolidin-3-yl)-1H-indole-2-carboxamide

The title compound was prepared according to Method A using (S)-tert-butyl 3-aminopyrrolidine-1-carboxylate (Aldrich) and 5-methoxy-1H-indole-2-carboxylic acid (Aldrich). ¹H NMR (300 MHz, MeOH-d₄) δ 1.84-1.96 (m, 1H), 2.17-2.31 (m, 1H), 2.93 (dd, J=11.9, 4.8 Hz, 1H), 2.97-3.08 (m, 1H), 3.11-3.20 (m, 1H), 3.25 (dd, J=11.9, 7.1 Hz, 1H), 3.81 (s, 3H), 4.48-4.53 (m, 1H), 6.88 (dd, J=8.9, 2.6 Hz, 1H), 7.03 (s; 1H), 7.06 (d, J=2.4 Hz, 1H), 7.32 (d, J=9.1 Hz, 1H) ppm; MS (DCI/NH₃) m/z 260 (M+H)⁺.

### Example 37

### (S)-5-fluoro-N-(1-methylpyrrolidin-3-yl)-1H-indole-2-carboxamide

The title compound was prepared according to Method C using the product of Example 35. ¹H NMR (300 MHz, MeOH-d₄) δ 1.79-1.99 (m, 1H), 2.29-2.45 (m, 4H), 2.50-2.68 (m, 2H), 2.76-2.86 (m, 1H), 2.89 (dd, J=10.3, 7.1 Hz, 1H), 4.51-4.69 (m, 1H), 6.99 (td, J=9.2, 2.6 Hz, 1H), 7.07 (d, J=0.8 Hz, 1H), 7.26 (dd, J=9.5, 2.4 Hz, 1H), 7.40 (dd, J=8.9, 4.6 Hz, 1H) ppm; MS (DCI/NH₃) m/z 262 (M+H)⁺.

### Example 38

### (S)-5-Methoxy-N-(1-methylpyrrolidin-3-yl)-1H-indole-2-carboxamide

The title compound was prepared according to Method C using the product of Example 36. ¹H NMR (300 MHz, MeOH-d₄) δ 1.77-1.97 (m, 1H), 2.27-2.39 (m, 1H), 2.41 (s, 3H), 2.51-2.60 (m, 1H), 2.62 (dd, J=10.0, 4.9 Hz, 1H), 2.77-2.86 (m, 1H), 2.88 (dd, J=10.2, 7.1 Hz, 1H), 3.81 (s, 3H), 4.46-4.70 (m, 1H), 6.88 (dd, J=8.8, 2.4 Hz, 1H), 7.02 (d, J=0.7 Hz, 1H), 7.06 (d, J=2.4 Hz, 1H), 7.32 (d, J=8.8 Hz, 1H) ppm; MS (DCI/NH₃) m/z 274 (M+H)⁺.

### Example 39

### (S)-5,6-dimethoxy-N-(pyrrolidin-3-yl)-1H-indole-2-carboxamide hydrochloride

The title compound was prepared according to Method A using (S)-tert-butyl 3-aminopyrrolidine-1-carboxylate (Aldrich) and 5,6-dimethoxy-1H-indole-2-carboxylic acid (VWR). ¹H NMR (3.00 MHz, MeOH-d₄) δ 2.14-2.27 (m, 1H), 2.34-2.49 (m, 1H), 3.32-3.47 (m, 2H), 3.51-3.64 (m, 2H), 3.84 (s, 3H), 3.87 (s, 3H), 4.54-4.64 (m, 1H), 6.97 (s, 1H), 7.04 (s, 1H), 7.08 (s, 1H) ppm; MS (DCI/NH₃) m/z 290 (M+H)⁺.

### Example 40

### (S)-4,6-dichloro-N-(1-methylpyrrolidin-3-yl)-1H-indole-2-carboxamide

The title compound was prepared according to Method C using the product of Example 3. ¹H NMR (300 MHz, MeOH-d₄) δ 2.14-2.33 (m, 1H), 2.46-2.63 (m, 1H), 2.90 (s, 3H), 3.18-3.27 (m, 1H), 3.36-3.44 (m, 1H), 3.47-3.62 (m, 2H), 4.55-4.70 (m, 1H), 7.13 (d, J=1.7 Hz, 1H), 7.23 (d, J=0.7 Hz, 1H), 7.43 (dd, J=1.5, 0.8 Hz, 1H) ppm; MS (DCI/NH₃) m/z 312 (M+H)⁺, 314 (M+H)⁺.

### Example 41

### (R)-N-(pyrrolidin-3-yl)-3H-benzo[e]indole-2-carboxamide trifluoroacetate

The title compound was prepared according to Method A using (R)-tert-butyl 3-aminopyrrolidine-1-carboxylate (Aldrich) and 3H-benzo[e]indole-2-carboxylic acid (Chembridge). ¹H NMR (300 MHz, MeOH-d₄) δ 2.19-2.32 (m, 1H), 2.39-2.53 (m, 1H), 3.37-3.49 (m, 2H), 3.55-3.67 (m, 2H), 4.64 (ddd, J=12.2, 7.0, 5.4 Hz, 1H), 7.37-7.45 (m, 1H), 7.50-7.59 (m, 2H), 7.63-7.68 (m, 1H), 7.71 (s, 1H), 7.88 (d, J=7.9 Hz, 1H), 8.21 (d, J=8.3 Hz, 1H) ppm; MS (DCI/NH₃) m/z 280 (M+H)⁺.

### Example 42

### (S)-N-(pyrrolidin-3-yl)-3H-benzo[e]indole-2-carboxamide trifluoroacetate

The title compound was prepared according to Method A using (S)-tert-butyl 3-aminopyrrolidine-1-carboxylate (Aldrich) and 3H-benzo[e]indole-2-carboxylic acid (Chembridge). ¹H NMR (300 MHz, MeOH-d₄) δ 2.15-2.34 (m, 1H), 2.37-2.54 (m, 1H), 3.36-3.48 (m, 2H), 3.54-3.71 (m, 2H), 4.53-4.73 (m, 1H), 7.41 (t, J=7.5 Hz, 1H), 7.50-7.69 (m, 3H), 7.71 (s, 1H), 7.88 (d, J=7.9 Hz, 1H), 8.21 (d, J=7.9 Hz, 1H) ppm; MS (DCI/NH₃) m/z 280 (M+H)⁺.

### Example 43

### (R)-4-(difluoromethoxy)-N-(pyrrolidin-3-yl)-1H-indole-2-carboxamide trifluoroacetate

The title compound was prepared according to Method A using (R)-tert-butyl 3-aminopyrrolidine-1-carboxylate (Aldrich) and 4-(difluoromethoxy)-1H-indole-2-carboxylic acid (Enamine). ¹H NMR (300 MHz, MeOH-d₄) δ 2.15-2.28 (m, 1H), 2.35-2.48 (m, 1H), 3.35-3.46 (m, 2H), 3.51-3.64 (m, 2H), 4.52-4.65 (m, 1H), 6.81 (dd, J=7.6, 0.8 Hz, 1H), 6.93 (t, J=74.6 Hz, 1H), 7.15-7.26 (m, 2H), 7.32 (dt, J=8.3, 1.0, 0.8 Hz, 1H) ppm; MS (DCI/NH₃) m/z 296 (M+H)⁺.

### Example 44

### (S)-4-(difluoromethoxy)-N-pyrrolidin-3-yl)-1H-indole-2-carboxamide trifluoroacetate

The title compound was prepared according to Method A using (S)-tert-butyl 3-aminopyrrolidine-1-carboxylate (Aldrich) and 4-(difluoromethoxy)-1H-indole-2-carboxylic acid (Enamine). ¹H NMR (300 MHz, MeOH-d₄) δ 2.03-2.30 (m, 1H), 2.33-2.57 (m, 1H), 3.35-3.48 (m, 2H), 3.50-3.67 (m, 2H), 4.45-4.72 (m, 1H), 6.81 (dd, J=7.5, 0.7 Hz, 1H), 6.93 (t, J=74.6 Hz, 1H), 7.15-7.27 (m, 2H), 7.29-7.38 (m, 1H) ppm; MS (DCI/NH₃) m/z 296 (M+H)⁺.

### Example 45

### (S)-7-fluoro-N-(pyrrolidin-3-yl)-1H-indole-2-carboxamide trifluoroacetate

The title compound was prepared according to Method A using (S)-tert-butyl 3-aminopyrrolidine-1-carboxylate (Aldrich) and 7-Fluoro-1H-indole-2-carboxylic acid (Matrix). ¹H NMR (300 MHz, MeOH-d₄) δ 2.10-2.31 (m, 1H), 2.33-2.56 (m, 1H), 3.32-3.48 (m, 2H), 3.50-3.65 (m, 2H), 4.47-4.73 (m, 1H), 6.90-7.09 (m, 2H), 7.17 (d, J=3.4 Hz, 1H), 7.41 (d, J=7.5 Hz, 1H) ppm; MS (DCI/NH₃) m/z 248 (M+H)⁺.

### Example 46

### (S)-6-tert-butyl-N-(pyrrolidin-3-yl)-1H-indole-2-carboxamide trifluoroacetate

The title compound was prepared according to Method A using (S)-tert-butyl 3-aminopyrrolidine-1-carboxylate (Aldrich) and 6-tert-butyl-1H-indole-2-carboxylic acid (Enamine). ¹H NMR (300 MHz, MeOH-d₄) δ 1.37 (s, 9H), 2.12-2.32 (m, 1H), 2.34-2.54 (m, 1H), 3.33-3.45 (m, 2H), 3.50-3.64 (m, 2H), 4.48-4.68 (m, 1H), 7.06 (d, J=1.0 Hz, 1H), 7.20 (dd, J=8.5, 1.7 Hz, 1H), 7.40-7.47 (m, 1H), 7.52 (d, J=8.8 Hz, 1H) ppm; MS (DCI/NH₃) m/z 286 (M+H)⁺.

### Example 47

### (R)-4-fluoro-N-(pyrrolidin-3-yl)-1H-indole-2-carboxamide trifluoroacetate

The title compound was prepared according to Method A using (R)-tert-butyl 3-aminopyrrolidine-1-carboxylate (Aldrich) and 4-fluoro-1H-indole-2-carboxylic acid (Enamine). ¹H NMR (300 MHz, MeOH-d₄) δ 2.10-2.32 (m, 1H), 2.33-2.52 (m, 1H), 3.34-3.48 (m, 2H), 3.50-3.73 (m, 2H), 4.51-4.74 (m, 1H), 6.75 (ddd, J=10.5, 7.5, 0.7 Hz, 1H), 7.09-7.30 (m, 3H) ppm. MS (DCI/NH₃) m/z 248 (M+H)⁺.

### Example 48

### (S)-4-fluoro-N-(pyrrolidin-3-yl)-1H-indole-2-carboxamide trifluoroacetate

The title compound was prepared according to Method A using (S)-tert-butyl 3-aminopyrrolidine-1-carboxylate (Aldrich) and 4-fluoro-1H-indole-2-carboxylic acid (Enamine). ¹H NMR (300 MHz, MeOH-d₄) δ 2.07-2.33 (m, 1H), 2.32-2.55 (m, 1H), 3.33-3.49 (m, 2H), 3.49-3.69 (m, 2-H), 4.49-4.71 (m, 1H), 6.75 (dd, J=10.3, 7.5 Hz, 1H), 7.08-7.31 (m, 3H) ppm; MS (DCI/NH₃) m/z 248 (M+H)⁺.

### Example 49

### (R)-6-(methylthio)-N-(pyrrolidin-3-yl)-1H-indole-2-carboxamide trifluoroacetate

The title compound was prepared according to Method A using (R)-tert-butyl 3-aminopyrrolidine-1-carboxylate (Aldrich) and 6-(methylthio)-1H-indole-2-carboxylic acid (Enamine). ¹H NMR (300 MHz, MeOH-d₄) δ 1.96-2.31 (m, 1H), 2.35-2.48 (m, 1H), 2.51 (s, 3H), 3.34-3.49 (m, 2H), 3.51-3.67 (m, 2H), 4.52-4.66, (m, 1H), 7.03 (dd, J=8.5, 1.7 Hz, 1H), 7.07 (d, J=0.7 Hz, 1H), 7.31-7.40 (m, 1H), 7.52 (dd, J=8.5, 0.7 Hz, 1H) ppm; MS (DCI/NH₃) m/z 276 (M+H)⁺.

### Example 50

### (S)-6-(methylthio)-N-(pyrrolidin-3-yl)-1H-indole-2-carboxamide trifluoroacetate

The title compound was prepared according to Method A using (S)-tert-butyl 3-aminopyrrolidine-1-carboxylate (Aldrich) and 6-(methylthio)-1H-indole-2-carboxylic acid (Enamine). ¹H NMR (300 MHz, MeOH-d₄) δ 2.11-2.29 (m, 1H), 2.33-2.49 (m, 1H), 2.51 (s, 3H), 3.34-3.47 (m, 2H), 3.50-3.67 (m, 2H), 4.49-4.68 (m, 1H), 7.02 (dd, J=8.5, 1.7 Hz, 1H), 7.08 (d, J=1.0 Hz, 1H), 7.28-7.39 (m, 1H), 7.51 (d, J=8.5 Hz, 1H) ppm; MS (DCI/NH₃) m/z 276 (M+H)⁺.

### Example 51

### (R)-3,5-dimethyl-N-(pyrrolidin-3-yl)-1H-indole-2-carboxamide trifluoroacetate

The title compound was prepared according to Method A using (R)-tert-butyl 3-aminopyrrolidine-1-carboxylate (Aldrich) and 3,5-dimethyl-1H-indole-2-carboxylic acid (Chembridge). ¹H NMR (300 MHz, MeOH-d₄) δ 2.07-2.29 (m, 1H), 2.39-2.51 (m, 1H), 2.42 (s, 3H), 2.53 (s, 3H), 3.33-3.46 (m, 2H), 3.48-3.73 (m, 2H), 4.48-4.68 (m, 1H), 7.09 (dd, J=8.5, 1.7 Hz, 1H), 7.25 (d, J=8.5 Hz, 1H), 7.37 (d, J=0.7 Hz, 1H) ppm; MS (DCI/NH₃) m/z 258 (M+H)⁺.

### Example 52

### (S)-3,5-dimethyl-N-(pyrrolidin-3-yl)-1H-indole-2-carboxamide trifluoroacetate

The title compound was prepared according to Method A using (S)-tert-butyl 3-aminopyrrolidine-1-carboxylate (Aldrich) and 3,5-dimethyl-1H-indole-2-carboxylic acid (Chembridge). ¹H NMR (300 MHz, MeOH-d₄) δ 2.12-2.29 (m, 1H), 2.38-2.52 (m, 1H), 2.42 (s, 3H), 2.53 (s, 3H), 3.33-3.47 (m, 2H), 3.51-3.67 (m, 2H), 4.48-4.71 (m, 1H), 7.09 (dd, J=8.5, 1.4 Hz, 1H), 7.26 (d, J=8.1 Hz, 1H), 7.38 (d, J=0.7 Hz, 1H) ppm; MS (DCI/NH₃) m/z 258 (M+H)⁺.

### Example 53

### (R)-4-chloro-N-(pyrrolidin-3-yl)-1H-indole-2-carboxamide trifluoroacetate

The title compound was prepared according to Method A using (R)-tert-butyl 3-aminopyrrolidine-1-carboxylate (Aldrich) and 4-chloro-1H-indole-2-carboxylic acid (Enamine). ¹H NMR (300 MHz, MeOH-d₄) δ 2.14-2.32 (m, 1H), 2.33-2.54 (m, 1H), 3.35-3.49 (m, 2H), 3.50-3.72 (m, 2H), 4.55-4.69 (m, 1H), 7.07-7.11 (dd, J=7.5, 0.7 Hz, 1H), 7.19 (t, J=7.8 Hz, 1H), 7.25 (d, J=0.7 Hz, 1H), 7.36-7.43 (m, 1H) ppm; MS (DCI/NH₃) m/z 264 (M+H)⁺, 266 (M+H)⁺.

### Example 54

### (S)-4-chloro-N-(pyrrolidin-3-yl)-1H-indole-2-carboxamide trifluoroacetate

The title compound was prepared according to Method A using (S)-tert-butyl 3-aminopyrrolidine-1-carboxylate (Aldrich) and 4-chloro-1H-indole-2-carboxylic acid (Enamine). ¹H NMR (300 MHz, MeOH-d₄) δ 2.09-2.31 (m, 1H), 2.34-2.56 (m, 1H), 3.36-3.47 (m, 2H), 3.49-3.67 (m, 2H), 4.48-4.69 (m, 1H), 7.09 (d, J=7.6 Hz 1H), 7.19 (t, J=7.9 Hz, 1H), 7.25 (s, 1H), 7.40 (d, J=8.3 Hz, 1H) ppm; MS (DCI/NH₃) m/z 264 (M+H)⁺, 266 (M+H)⁺.

### Example 55

### (R)-4-methyl-N-pyrrolidin-3-yl)-1H-indole-2-carboxamide

The title compound was prepared according to Method A using (R)-tert-butyl 3-aminopyrrolidine-1-carboxylate (Aldrich) and 4-methyl-1H-indole-2-carboxylic acid (Enamine). ¹H NMR (300 MHz, MeOH-d₄) δ 1.80-2.07 (m, 1H), 2.11-2.35 (m, 1H), 2.53 (s, 3H), 2.98 (dd, J=11.9, 4.7 Hz, 1H), 3.02-3.11 (m, 1H), 3.14-3.28 (m, 2H), 4.42-4.63 (m, 1H), 6.82-6.90 (m, 1H), 7.10 (dd, J=8.1, 7.1 Hz, 1H), 7.19 (d, J=0.7 Hz, 1H), 7.25 (d, J=8.5 Hz, 1H) ppm; MS (DCI/NH₃) m/z 244 (M+H)⁺.

### Example 56

### (S)-4-methyl-N-(pyrrolidin-3-yl)-1H-indole-2-carboxamide

The title compound was prepared according to Method A using (S)-tert-butyl 3-aminopyrrolidine-1-carboxylate (Aldrich) and 4-methyl-1H-indole-2-carboxylic acid (Enamine). ¹H NMR (300 MHz, MeOH-d₄) δ 1.87-2.08 (m, 1H), 2.14-2.40 (m, 1H), 2.53 (s, 3H), 2.98 (dd, J=11.7, 4.6 Hz, 1H), 3.02-3.12 (m, 1H), 3.15-3.29 (m, 2H), 4.42-4.63 (m, 1H), 6.85 (d, J=7.1 Hz, 1H), 7.10 (t, J=7.5 Hz, 1H), 7.19 (s, 1H), 7.25 (d, J=8.3 Hz, 1H) ppm; MS (DCI/NH₃) m/z 244 (M+H)⁺.

### Example 57

### (R)-3-methyl-N-(pyrrolidin-3-yl)-1H-indole-2-carboxamide trifluoroacetate

The title compound was prepared according to Method A using (R)-tert-butyl 3-aminopyrrolidine-1-carboxylate (Aldrich) and 3-methyl-1H-indole-2-carboxylic acid (Matrix). ¹H NMR (300 MHz, MeOH-d₄) δ 2.08-2.30 (m, 1H), 2.35-2.52 (m, 1H), 2.56 (s, 3H), 3.34-3.50 (m, 2H), 3.49-3.71 (m, 2H), 4.49-4.77 (m, 1H), 7.07 (t, J=7.5 Hz, 1H), 7.17-7.31 (m, 1H), 7.37 (d, 1H), 7.60 (d, J=7.9 Hz, 1H) ppm; MS (DCI/NH₃) m/z 244 (M+H)⁺.

### Example 58

### (S)-3-methyl-N-(pyrrolidin-3-yl)-1H-indole-2-carboxamide trifluoroacetate

The title compound was prepared according to Method A using (S)-tert-butyl 3-aminopyrrolidine-1-carboxylate (Aldrich) and 3-methyl-1H-indole-2-carboxylic acid (Matrix). ¹H NMR (300 MHz, MeOH-d₄) δ 2.11-2.30 (m, 1H), 2.36-2.51 (m, 1H), 2.56 (s, 3H), 3.34-3.46 (m, 2H), 3.51-3.72 (m, 2H), 4.51-4.69 (m, 1H), 7.02-7.14 (m, 1H), 7.18-7.29 (m, 1H), 7.37 (dt, J=8.1, 1.0 Hz, 1H), 7.61 (dt, J=8.1, 1.0 Hz, 1H) ppm; MS (DCI/NH₃) m/z 244 (M+H)⁺.

### Example 59

### (R)-N-(1-methylpyrrolidin-3-yl)-3H-benzo[e]indole-2-carboxamide

The title compound was prepared according to Method C using the product of Example 41. ¹H NMR (300 MHz, MeOH-d₄) δ 1.80-2.11 (m, 1H), 2.33-2.47 (m, 1H), 2.49 (s, 3H), 2.60-2.72 (m, 1H), 2.76 (dd, J=10.3, 4.9 Hz, 1H), 2.88-3.05 (m, 2H), 4.44-4.76 (m, 1H), 7.35-7.44 (m, 1H), 7.49-7.66 (m, 3H), 7.70 (s, 1H), 7.87 (d, J=8.1 Hz, 1H), 8.21 (d, J=8.1 Hz, 1H) ppm; MS (DCI/NH₃) m/z 294 (M+H)⁺.

### Example 60

### (R)-6-chloro-N-(pyrrolidin-3-yl)-1H-indole-2-carboxamide trifluoroacetate

The title compound was prepared according to Method A using (R)-tert-butyl 3-aminopyrrolidine-1-carboxylate (Aldrich) and 6-chloro-1H-indole-2-carboxylic acid (Matrix). ¹H NMR (300 MHz, MeOH-d₄) δ 2.11-2.29 (m, 1H), 2.34-2.53 (m, 1H), 3.34-3.46 (m, 2H), 3.51-3.71 (m, 2H), 4.52-4.69 (m, 1H), 7.06 (dd, J=8.6, 1.9 Hz, 1H), 7.11 (d, J=1.0 Hz, 1H), 7.44-7.48 (m, 4H), 7.58 (d, J=8.1 Hz, 1H) ppm; MS (DCI/NH₃) m/z 264 (M+H)⁺, 266 (M+H)⁺.

### Example 61

### (S)-6-chloro-N-(pyrrolidin-3-yl)-1H-indole-2-carboxamide trifluoroacetate

The title compound was prepared according to Method A using (S)-tert-butyl 3-aminopyrrolidine-1-carboxylate (Aldrich) and 6-chloro-1H-indole-2-carboxylic acid (Matrix). ¹H NMR (300 MHz, MeOH-d₄) δ 2.10-2.30 (m, 1H), 2.34-2.57 (m, 1H), 3.34-3.46 (m, 2H), 3.50-3.67 (m, 2H), 4.47-4.69 (m, 1H), 7.05 (dd, J=8.7, 1.6 Hz, 1H), 7.11 (s, 1H), 7.46 (s, 1H), 7.58 (d, J=8.3 Hz, 1H) ppm; MS (DCI/NH₃) m/z 264 (M+H)⁺, 266 (M+H)⁺.

### Example 62

(S)-N-(1-methylpyrrolidin-3-yl)-3H-benzo[e]indole-2-carboxamide

The title compound was prepared according to Method C using the product of Example 42. ¹H NMR (300 MHz, MeOH-d₄) δ 1.74-2.03 (m, 1H), 2.31-2.43: (m, 1H), 2.44 (s, 3H), 2.55-2.73 (m, 2H), 2.79-2.89 (m, 1H), 2.94 (dd, J=10.0, 7.3 Hz, 1H), 4.51-4.73 (m, 1H), 7.34-7.47 (m, 1H), 7.47-7.67 (m, 3H), 7.70 (d, J=1.0 Hz, 1H), 7.87 (d, J=7.8 Hz, 1H), 8.21 (d, J=8.5 Hz, 1H) ppm; MS (DCI/NH₃) m/z 294 (M+H)⁺.

### Example 63

### (R)-6-bromo-N-(pyrrolidin-3-yl)-1H-indole-2-carboxamide trifluoroacetate

The title compound was prepared according to Method A using (R)-tert-butyl 3-aminopyrrolidine-1-carboxylate (Aldrich) and 6-bromo-1H-indole-2-carboxylic acid (Asymchem). ¹H NMR (300 MHz, MeOH-d₄) δ 2.09-2.31 (m, 1H), 2.36-2.55 (m, 1H), 3.33-3.49 (m, 2H), 3.49-3.71 (m, 2H), 4.46-4.70 (m, 1H), 7.11 (d, J=1.0 Hz, 1H), 7.19 (dd, J=8.5, 1.7 Hz, 1H), 7.53 (d, J=8.5 Hz, 1H), 7.61-7.64 (m, 1H) ppm; MS (DCI/NH₃) m/z 308 (M+H)⁺, 310 (M+H)⁺.

### Example 64

(S)-bromo-N-(pyrrolidin-3-yl)-1H-indole-2-carboxamide trifluoroacetate

The title compound was prepared according to Method A using (S)-tert-butyl 3-aminopyrrolidine-1-carboxylate (Aldrich) and 6-bromo-1H-indole-2-carboxylic acid (Asymchem). ¹H NMR (300 MHz, MeOH-d₄) δ 2.07-2.31 (m, 1H), 2.32-2.57 (m, 1H), 3.33-3.49 (m, 2H), 3.49-3.68 (m, 2H), 4.51-4.69 (m, 1H), 7.11 (s, 1H), 7.19 (dd, J=8.5, 1.8 Hz, 1H), 7.53 (d, J=8.3 Hz, 1H), 7.63 (s, 1H) ppm; MS (DCI/NH₃) m/z 308 (M+H)⁺, 310 (M+H)⁺.

### Example 65

### (S)-7-methyl-N-(pyrrolidin-3-yl)-1H-indole-2-carboxamide trifluoroacetate

The title compound was prepared according to Method A using (S)-tert-butyl-3-aminopyrrolidine-1-carboxylate (Aldrich) and 7-methyl-1H-indole-2-carboxylic acid (Aldrich). ¹H NMR (300 MHz, MeOH-d₄) δ 2.07-2.32 (m, 1H), 2.37-2.50 (m, 1H), 2.52 (s, 3H), 3.35-3.49 (m, 2H), 3.51-3.69 (m, 2H), 4.51-4.71 (m, 1H), 6.93-7.07 (m, 2H), 7.13 (s, 1H), 7.44 (d, J=7.8 Hz, 1H) ppm; MS (DCI/NH₃) m/z 244 (M+H)⁺.

### Example 66

### (R)-7-methyl-N-(pyrrolidin-3-yl)-1H-indole-2-carboxamide trifluoroacetate

The title compound was prepared according to Method A using (R)-tert-butyl 3-aminopyrrolidine-1-carboxylate (Aldrich) and 7-methyl-1H-indole-2-carboxylic acid (Aldrich). ¹H NMR (300 MHz, MeOH-d₄) δ 2.12-2.30 (m, 1H), 2.34-2.50 (m, 1H), 2.52 (s, 3H), 3.35-3.48 (m, 2H), 3.53-3.76 (m, 2H), 4.53-4.71 (m, 1H), 6.94-7.08 (m, 2H), 7.13 (s, 1H), 7.44 (d, J=7.1 Hz, 1H) ppm; MS (DCI/NH₃) m/z 244 (M+H)⁺.

### Example 67

### (R)-4-(difluoromethoxy)-N-(1-methylpyrrolidin-3-yl)-1H-indole-2-carboxamide

The title compound was prepared according to Method C using the product of Example 43. ¹H NMR (300 MHz, MeOH-d₄) δ 1.78-1.98 (m, 1H), 2.29-2.39 (m, 1H), 2.41 (s, 3H), 2.50-2.68 (m, 2H), 2.76-2.94 (m, 2H), 4.45-4.69 (m, 1H), 6.80 (d, J=7.5 Hz, 1H), 6.90 (t, J=74.6 Hz, 1H), 7.13-7.25 (m, 2H), 7.31 (d, J=7.5 Hz, 1H) ppm; MS (DCI/NH₃) m/z 310 (M+H)⁺.

### Example 68

### (S)-4-(difluoromethoxy)-N-(1-methylpyrrolidin-3-yl)-1H-indole-2-carboxamide

The title compound was prepared according to Method C using the product of Example 44. ¹H NMR (300 MHz, MeOH-d₄) δ 1.75-1.98 (m, 1H), 2.29-2.39 (m, 1H) 2.41 (s, 3H), 2.51-2.69 (m, 2H), 2.75-2.97 (m, 2H), 4.50-4.68 (m, 1H), 6.80 (dd, J=7.8, 0.7 Hz, 1H), 6.90 (t, J=74.6 Hz, 1H), 7.11-7.25 (m, 2H), 7.31 (dt, J=8.3, 0.7 Hz, 1H) ppm; MS (DCI/NH₃) m/z 310 (M+H)⁺.

### Example 69

### (S)-5-chloro-N-(pyrrolidin-3-yl)-1H-indole-2-carboxamide

The title compound was prepared according to Method A using (S)-tert-butyl 3-aminopyrrolidine-1-carboxylate (Aldrich) and 5-chloro-1H-indole-2-carboxylic acid (Aldrich). ¹H NMR (300 MHz, MeOH-d₄) δ 1.81-2.04 (m, 1H), 2.15-2.36 (m, 1H), 2.86-3.08 (m, 2H), 3.10-3.29 (m, 2H), 4.42-4.59 (m, 1H), 7.06 (d, J=1.0 Hz, 1H), 7.18 (dd, J=8.6, 2.2 Hz, 1H), 7.41 (dd, J=8.8, 0.7 Hz, 1H), 7.59 (d, J=2.0 Hz, 1H) ppm; MS (DCI/NH₃) m/z 264 (M+H)⁺, 266 (M+H)⁺.

### Example 70

### (R)-3-methyl-N-(1-methylpyrrolidin-3-yl)-1H-indole-2-carboxamide

The title compound was prepared according to Method C using the product of Example 57. ¹H NMR (300 MHz, MeOH-d₄) δ 1.76-1.98 (m, 1H), 2.31-2.45 (m, 1H), 2.42 (s, 3H), 2.48-2.56 (m, 1H), 2.55 (s, 3H), 2.68 (dd, J=10.2, 4.4 Hz, 1H), 2.81-2.93 (m, 2H), 4.45-4.70 (m, 1H), 7.06 (ddd, J=8.1, 6.9, 1.0 Hz, 1H), 7.16-7.27 (m, 1H), 7.36 (d, J=8.5 Hz, 1H), 7.59 (d, J=8.1 Hz, 1H) ppm; MS (DCI/NH₃) m/z 258 (M+H)⁺.

### Example 71

### (S)-3-methyl-N-(1-methylpyrrolidin-3-yl)-1H-indole-2-carboxamide

The title compound was prepared according to Method C using the product of Example 58. ¹H NMR (300 MHz, MeOH-d₄) δ 1.71-1.97 (m, 1H), 2.32-2.41 (m, 1H), 2.42 (s, 3H), 2.45-2.54 (m, 1H), 2.55 (s, 3H), 2.68 (dd, J=10.2, 4.4 Hz, 1H), 2.81-2.94 (m, 2H), 4.46-4.68 (m, 1H), 7.06 (ddd, J=8.0, 7.0, 1.0 Hz, 1H), 7.22 (ddd, J=8.2, 7.0, 1.4 Hz, 1H), 7.31-7.38 (m, 1H), 7.59 (dt, J=8.1, 1.0 Hz, 1H) ppm; MS (DCI/NH₃) m/z 258 (M+H)⁺.

### Example 72

### (R)-4-chloro-N-(1-methylpyrrolidin-3-yl)-1H-indole-2-carboxamide

The title compound was prepared according to Method C using the product of Example 53. ¹H NMR (300 MHz, MeOH-d₄) δ 1.63-2.01 (m, 1H), 2.30-2.40 (m, 1H), 2.41 (s, 3H), 2.51-2.70 (m, 2H), 2.76-2.87 (m, 1H), 2.90 (dd, J=10.3, 7.1 Hz, 1H), 4.49-4.66 (m, 1H), 7.05-7.11 (m, 1H), 7.17 (t, J=7.9 Hz, 1H), 7.23 (s, 1H), 7.38 (dd, J=8.3, 0.8 Hz, 1H) ppm; MS (DCI/NH₃) m/z 278 (M+H)⁺, 280 (M+H)⁺.

### Example 73

### (S)-4-chloro-N-(1-methylpyrrolidin-3-yl)-1H-indole-2-carboxamide

The title compound was prepared according to Method C using the product of Example 54. ¹H NMR (300 MHz, MeOH-d₄) δ 1.76-1.99 (m, 1H), 2.28-2.40 (m, 1H), 2.42 (s, 3H), 2.51-2.69 (m, 2H), 2.77-2.87 (m, 1H), 2.90 (dd, J=10.3, 7.1 Hz, 1H), 4.50-4.66 (m, 1H), 7.08 (d, J=7.9 Hz 1H), 7.17 (t, J=7.9 Hz, 1H), 7.23 (s, 1H), 7.38 (d, J=7.9 Hz, 1H) ppm; MS (DCI/NH₃) m/z 278 (M+H)⁺, 280 (M+H)⁺.

### Example 74

### (R)-6-chloro-N-(1-methylpyrrolidin-3-yl)-1H-indole-2-carboxamide

The title compound was prepared according to Method C using the product of Example 60. ¹H NMR (300 MHz, MeOH-d₄) δ 1.76-1.98 (m, 1H), 2.30-2.39 (m, 1H), 2.41 (s, 1H), 2.51-2.67 (m, 2H), 2.76-2.86 (m, 1H), 2.89 (dd, J=10.3, 7.1 Hz, 1H), 4.47-4.67 (m, 1H), 7.04 (dd, J=8.3, 2.0 Hz, 1H), 7.10 (s, 1H), 7.41-7.47 (m, 1H), 7.57 (d, J=8.7 Hz, 1H) ppm; MS (DCI/NH₃) m/z 278 (M+H)⁺, 280 (M+H)⁺.

### Example 75

### (R)-5-methyl-N-(pyrrolidin-3-yl)-1H-indole-2-carboxamide trifluoroacetate

The title compound was prepared according to Method A using (R)-tert-butyl 3-aminopyrrolidine-1-carboxylate (Aldrich) and 5-methyl-1H-indole-2-carboxylic acid (VWR). ¹H NMR (300 MHz, MeOH-d₄) δ 2.11-2.29 (m, 1H), 2.40 (s, 3H), 2.41-2.51 (m, 1H), 3.33-3.51 (m, 2H), 3.50-3.65 (m, 2H), 4.46-4.72 (m, 1H), 7.02 (d, J=0.7 Hz, 1H), 7.07 (dd, J=8.5, 1.7 Hz, 1H), 7.32 (d, J=8.5 Hz, 1H), 7.38, (s, 1H) ppm; MS (DCI/NH₃) m/z 244 (M+H)⁺.

### Example 76

### (S)-5-bromo-N-(pyrrolidin-3-yl)-1H-indole-2-carboxamide trifluoroacetate

The title compound was prepared according to Method A using (S)-tert-butyl 3-aminopyrrolidine-1-carboxylate (Aldrich) and 5-bromo-1H-indole-2-carboxylic acid (Aldrich). ¹H NMR (300 MHz, MeOH-d₄) δ 2.07-2.32 (m, 1H), 2.35-2.53 (m, 1H), 3.33-3.49 (m, 2H), 3.49-3.68 (m, 2H), 4.45-4.71 (m, 1H), 7.07 (s, 1H), 7.27-7.41 (m, 2H), 7.77 (d, J=1.6 Hz, 1H) ppm; MS (DCI/NH₃) m/z 308 (M+H)⁺, 310 (M+H)⁺.

### Example 77

### (R)-4,6,7-trimethyl-N-(pyrrolidin-3-yl)-1H-indole-2-carboxamide trifluoroacetate

The title compound was prepared according to Method A using (R)-tert-butyl 3-aminopyrrolidine-1-carboxylate (Aldrich) and 4,6,7-trimethyl-1H-indole-2-carboxylic acid (Enamine). ¹H NMR (300 MHz, MeOH-d₄) δ 2.14-2.29 (m, 1H), 2.34 (s, 3H), 2.38-2.50 (m, 1H), 2.40 (s, 3H), 2.45 (s, 3H), 3.34-3.49 (m, 2H), 3.50-3.71 (m, 2H), 4.49-4.69 (m, 1H), 6.72 (s, 1H), 7.16 (s, 1H); MS (DCI/NH₃) m/z 272 (M+H)⁺.

### Example 78

### (S)-4,6,7-trimethyl-N-(pyrrolidin-3-yl)-1H-indole-2-carboxamide trifluoroacetate

The title compound was prepared according to Method A using (S)-tert-butyl 3-aminopyrrolidine-1-carboxylate (Aldrich) and 4,6,7-trimethyl-1H-indole-2-carboxylic acid (Enamine). ¹H NMR (300 MHz, MeOH-d₄) δ 2.14-2.28 (m, 1H), 2.34 (s, 3H), 2.40 (s, 3H), 2.41-2.50 (m, 1H), 2.45 (s, 3H), 3.34-3.50 (m, 2H), 3.51-3.70 (m, 2H), 4.49-4.71 (m, 1H), 6.72 (s, 1H), 7.16 (s, 1H) ppm; MS (DCI/NH₃) m/z 272 (M+H)⁺.

### Example 79

### (R)-4,7-dimethoxy-N-(pyrrolidin-3-yl)-1H-indole-2-carboxamide trifluoroacetate

The title compound was prepared according to Method A using (R)-tert-butyl 3-aminopyrrolidine-1-carboxylate (Aldrich) and 4,7-dimethoxy-1H-indole-2-carboxylic acid (Enamine). ¹H NMR (300 MHz, MeOH-d₄) δ 2.06-2.32 (m, 1H), 2.34-2.58 (m, 1H), 3.34-3.49 (m, 2H), 3.50-3.73 (m, 2H), 3.88 (s, 3H), 3.92 (s, 3H), 4.49-4.66 (m, 1H), 6.40 (d, J=8.1 Hz, 1H), 6.63 (d, J=8.5 Hz, 1H), 7.19 (s, 1H) ppm; MS (DCI/NH₃) m/z 290 (M+H)⁺.

### Example 80

### (S)-6-chloro-N-(1-methylpyrrolidin-3-yl)-1H-indole-2-carboxamide

The title compound was prepared according to Method C using the product of Example 61. ¹H NMR (300 MHz, MeOH-d₄) δ 1.72-1.98 (it, 1H), 2.28-2.39 (m, 1H), 2.41 (s, 3H), 2.52-2.66 (m, 2H), 2.76-2.86 (m, 1H), 2.90 (dd, J=9.9, 7.1 Hz, 1H), 4.47-4.64 (m, 1H), 7.04 (dd, J=8.5, 1.8 Hz, 1H), 7.10 (s, 1H), 7.45 (s, 1H), 7.57; (d, J=8.3 Hz, 1H) ppm; MS (DCI/NH₃) m/z 278 (M+H), 280 (M+H)⁺.

### Example 81

### (S)-4,7-dimethoxy-N-(pyrrolidin-3-yl)-1H-indole-2-carboxamide trifluoroacetate

The title compound was prepared according to Method A using (S)-tert-butyl 3-aminopyrrolidine-1-carboxylate (Aldrich) and 4,7-dimethoxy-1H-indole-2-carboxylic acid (Enamine). ¹H NMR-(300 MHz, MeOH-d₄) δ 2.10-2.311 (m, 1H), 2.33-2.57 (m, 1H), 3.33-3.45 (m, 2H), 3.50-3.65 (m, 2H), 3.87 (s, 3H), 3.92 (s, 3H), 4.47-4.6.7 (m, 1. H), 6.39 (d, J=8.3 Hz, 1H), 6.63 (d, J=8.3 Hz, 1H), 7.19 (s, 1H) ppm; MS (DCI/NH₃) m/z 290 (M+H)⁺.

### Example 82

### (R)-6-bromo-N-(1-methylpyrrolidin-3-yl)-1H-indole-2-carboxamide

The title compound was prepared according to Method C using the product of Example 63. ¹H NMR (300 MHz, MeOH-d₄) δ 1.74-1.98 (m, 1H), 2.29-2.39 (m, 1H), 2.4.1 (s, 3H), 2.50-2.68 (m, 2H), 2.75-2.85 (m, 1H), 2.89 (dd, J=1.0.3, 7.1 Hz, 1H), 4.50-4.64 (m, 1H), 7.09 (s, 1H), 7.17 (dd, J=8.5, 1.8 Hz, 1H), 7.52 (d, J=8.3 Hz, 1H), 7.61 (s, 1H) ppm; MS (DCI/NH₃) m/z 322 (M+H)⁺, 324 (M+H)⁺.

### Example 83

### (R)-7-methyl-N-(1-methylpyrrolidin-3-yl)-1H-indole-2-carboxamide

The title compound was prepared according to Method C using the product of Example 66. ¹H NMR (300 MHz, MeOH-d4) δ 1.78-2.05 (m, 1H), 2.30-2.41 (m, 1H), 2.42 (s, 3H), 2.52 (s, 3H), 2.54-2.70 (m, 2H), 2.76-2.86 (m, 1H), 2.90 (dd, J=10.3, 7.1 Hz, 1H), 4.53-4.65 (m, 1H), 6.92-7.07 (m, 2H), 7.11 (s, 1H), 7.43 (d, J=7.5 Hz, 1H) ppm; MS (DCI/NH₃) m/z 258 (M+H)⁺.

### Example 84

### (S)-7-methyl-N-(1-methylpyrrolidin-3-yl)-1H-indole-2-carboxamide

The title compound was prepared according to Method C using the product of Example 65. ¹H NMR (300 MHz, MeOH-d₄) δ ppm 1.77-2.01 (m, 1H), 2.29-2.40 (m, 1H), 2.42 (s, 3H), 2.52 (s, 3H), 2.54-2.69 (m, 2H), 2.77-2.86 (m, 1H), 2.90 (dd, J=9.9, 7.1 Hz, 1H), 4.49-4.67 (m, 1H), 6.92-7.06 (m, 2H): 7.11 (s, 1H), 7.43 (d, J=7.5 Hz, 1H) ppm; MS (DCI/NH₃) m/z 258 (M+H)⁺.

### Example 85

### (R)-5-methyl-N-(1-methylpyrrolidin-3-yl)-1H-indole-2-carboxamide

The title compound was prepared according to Method C using the product of Example 75. ¹H NMR (300 MHz, MeOH-d₄) δ 1.74-1.99 (m, 1H), 2.29-2.38 (m, 1H), 2.40 (s, 3H), 2.41 (s, 3H), 2.52-2.68 (m, 2H), 2.75-2.95 (m, 2H), 4.47-4.66 (m, 1H), 7.00 (d, J=1.0 Hz, 1H), 7.05 (dd, J=8.6, 1.5 Hz, 1H), 7.31 (d, J=8.5 Hz, 1H), 7.36-7.39 (m, 1H) ppm; MS (DCI/NH₃) m/z 258 (M+H)⁺.

### Example 86

### (S)-5-bromo-N-(1-methylpyrrolidin-3-yl)-1H-indole-2-carboxamide

The title compound was prepared according to Method C using the product of Example 76. ¹H NMR (300 MHz, MeOH-d₄) δ 1.73-2.04 (m, 1H), 2.30-2.39 (m, 1H), 2.41 (s, 3H), 2.52-2.60 (m, 1H), 2.62 (dd, J=10.3, 5.3 Hz, 1H), 2.75-2.86 (m, 1H), 2.89 (dd, J=10.2, 7.1 Hz, 1H), 4.46-4.68 (m, 1H), 7.05 (s, 1H), 7.25-7.43 (m, 2 M), 7.75 (d, J=1.4 Hz, 1H) ppm; MS (DCI/NH₃) m/z 322 (M+H)⁺, 324 (M+H)⁺.

### Example 87

### (R)-4,6,7-trimethyl-N-1-methylpyrrolidin-3-yl)-1H-indole-2-carboxamide

The title compound was prepared according to Method C using the product of Example 77. ¹H NMR (300 MHz, MeOH-d₄) δ 1.80-1.97 (m, 1H), 2.35-2.41 (m, 1H), 2.39 (s, 3H), 2.41 (s, 3H), 2.45 (s, 3H), 2.51-2.60 (m, 1H), 2.63 (dd, J=9.7, 4.6 Hz, 1H), 2.77-2.97 (m, 2H), 4.48-4.71 (m, 1H), 6.71 (s, 1H), 7.14 (s, 1H) ppm; MS (DCI/NH₃) m/z 286 (M+H)⁺.

### Example 88

### (S)-4,6,7-trimethyl-N-(1-methylpyrrolidin-3-yl)-1H-indole-2-carboxamide

The title compound was prepared according to Method C using the product of Example 78. ¹H NMR (300 MHz, MeOH-d₄) δ 1.75-2.03 (m, 1H), 2.32-2.38 (m, 1H), 2.33 (s, 3H), 2.39 (s, 3H), 2.41 (s, 3H), 2.45 (s, 3H), 2.51-2.60 (m, 1H), 2.63 (dd, J=10.1, 4.6 Hz, 1H), 2.77-2.86 (m, 1H), 2.89 (dd, J=10.1, 6.9 Hz, 1H), 4.50-4.67 (m, 1H), 6.70 (s, 1H), 7.14 (s, 1H) ppm; MS (DCI/NH₃) m/z 286 (M+H)⁺.

### Example 89

### (R)-4,7-dimethoxy-N-(1-methylpyrrolidin-3-yl)-1H-indole-2-carboxamide

The title compound was prepared according to Method C using the product of Example 79. ¹H NMR (300 MHz, MeOH-d₄) δ 1.74-1.97 (m, 1H), 2.28-2.40 (m, 1H), 2.41 (s, 3H), 2.51-2.61 (m, 1H), 2.63 (dd, J=10.2, 4.7 Hz, 1H), 2.77-2.85 (m, 1H), 2.89 (dd, J=10.2, 7.1 Hz, 1H), 3.88 (s, 3H), 3.92 (s, 3H), 4.47-4.64 (m, 1H), 6.38 (d, J=8.1 Hz, 1H), 6.61 (d, J=8.1 Hz, 1H), 7.17 (s, 1H) ppm; MS (DCI/NH₃) m/z 304 (M+H)⁺.

### Example 90

### (S)-4,7-dimethoxy-N-(1-methylpyrrolidin-3-yl)-1H-indole-2-carboxamide

The title compound was prepared according to Method C using the product of Example 81. ¹H NMR (300 MHz, MeOH-d₄) δ 1.73-1.99 (m, 1H), 2.26-2.39 (m, 1H), 2.41 (s, 3H), 2.51-2.60 (m, 1H), 2.63 (dd, J=10.0, 4.9 Hz, 1H), 2.74-2.85 (m, 1H), 2.89 (dd, J=10.2, 7.1 Hz, 1H), 3.88 (s, 3H), 3.92 (s, 3H), 4.42-4.67 (m, 1H), 6.38 (d, J=8.5 Hz, 1H), 6.61 (d, J=8.5 Hz, 1H), 7.17 (s, 1H) ppm; MS (DCI/NH₃) m/z 304 (M+H)⁺.

### Example 91

### (R)-4,7-dimethyl-N-(pyrrolidin-3-yl)-1H-indole-2-carboxamide

The title compound was prepared according to Method A using (R)-tert-butyl 3-aminopyrrolidine-1-carboxylate (Aldrich) and 4,7-dimethyl-1H-indole-2-carboxylic acid (Enamine). ¹H NMR (300 MHz, MeOH-d₄) δ 1.70-2.07 (m, 1H), 2.12-2.37 (m, 1H), 2.48 (s, 3H), 2.49 (s, 3H), 2.96 (dd, J=11.9, 4.8 Hz, 1H), 3.00-3.09 (m, 1H), 3.11-3.28 (m, 2H), 4.43-4.64 (m, 1H), 6.77 (d, J=7.1 Hz, 1H), 6.91 (d, J=7.1 Hz, 1H), 7.20 (s, 1H) ppm; MS (DCI/NH₃) m/z 258 (M+H)⁺.

### Example 92

### (R)-6-bromo-4-fluoro-N-(pyrrolidin-3-yl)-1H-indole-2-carboxamide

The title compound was prepared according to Method A using (R)-tert-butyl 3-aminopyrrolidine-1-carboxylate (Aldrich) and 6-bromo-4-fluoro-1H-indole-2-carboxylic acid (Enamine). ¹H NMR (300 MHz, MeOH-d₄) δ 1.77-2.01 (m, 1H), 2.11-2.35 (m, 1H), 2.91 (dd, J=11.7, 5.0 Hz, 1H), 2.96-3.06 (m, 1H), 3.09-3.19 (m, 1H), 3.23 (dd, J=11.9, 6.7 Hz, 1H), 4.35-4.64 (m, 1H), 6.94 (dd, J=9.9, 1.6 Hz, 1H), 7.18 (s, 1H), 7.46 (s, 1H) ppm; MS (DCI/NH₃) m/z 326, 328 (M+H)⁺.

### Example 93

### (R)-6-fluoro-7-methyl-N-(pyrrolidin-3-yl)-1H-indole-2-carboxamide

The title compound was prepared according to Method A using (R)-tert-butyl 3-aminopyrrolidine-1-carboxylate (Aldrich) and 6-fluoro-7-methyl-1H-indole-2-carboxylic acid (Matrix). ¹H NMR (300 MHz, MeOH-d₄) δ 1.81-2.01 (m, 1H), 2.14-2.33 (m, 1H), 2.43 (d, J=1.7 Hz, 3H), 2.95 (dd, J=11.9, 4.7 Hz, 1H), 2.99-3.10 (m, 1H), 3.12-3.28 (m, 2H), 4.41-4.63 (m, 1H), 6.84 (dd, J=10.5, 8.8 Hz, 1H), 7.11 (s, 1H), 7.41 (dd, J=8.8, 5.1 Hz, 1H) ppm; MS (DCI/NH₃) m/z 262 (M+H)⁺.

### Example 94

### (R)-4,7-dimethyl-N-(1-methylpyrrolidin-3-yl)-1H-indole-2-carboxamide trifluoroacetate

The title compound was prepared according to Method C using the product of Example 91. ¹H NMR (300 MHz, MeOH-d₄) δ 2.16-2.38 (m, 1H), 2.48 (s, 3H), 2.49 (s, 3H), 2.56-2.81 (m, 1H), 3.01 (s, 3H), 3.13-3.42 (m, 2H), 3.67-4.15 (m, 2H)-4.50-4.72 (m, 1H), 6.78 (d, J=7.1 Hz, 1H), 6.93 (d, J=7.8 Hz, 1H), 7.19 (s, 1H) ppm; MS (DCI/NH₃) m/z 272 (M+H)⁺.

### Example 95

### (R)-6-bromo-4-fluoro-N-(1-methylpyrrolidin-3-yl)-1H-indole-2-carboxamide

The title compound was prepared according to Method C the product of Example 92. ¹H NMR (300 MHz, MeOH-d₄) δ 1.78-1.99 (m, 1H), 2.27-2.39 (m, 1H), 2.41 (s, 3H), 2.52-2.68 (m, 2H), 2.81 (td, J=8.9, 5.9 Hz, 1H), 2.91 (dd, J=10.2, 7.1 Hz, 1H), 4.46-4.67 (m, 1H), 6.94 (dd, J=9.8, 1.4 Hz, 1H), 7.18 (d, J=1.0 Hz, 1H), 7.42-7.48 (m, 1H) ppm; MS (DCI/NH₃) m/z 340 (M−H)⁺, 342 (M+H)⁺.

### Example 96

### (R)-5-trifluoromethoxy-N-(pyrrolidin-3-yl)-1H-indole-2-carboxamide trifluoroacetate

The title compound was prepared according to Method A using (R)-tert-butyl 3-aminopyrrolidine-1-carboxylate (Aldrich) and 5-trifluoromethoxy-1H-indole-2-carboxylic acid (Maybridge). ¹H NMR (300 MHz, MeOH-d₄) δ 2.12-2.33 (m, 1H), 2.33-2.56 (m, 1H), 3.34-3.49 (m, 2H), 3.50-3.68 (m, 2H), 4.44-4.72 (m, 1H), 7.09-7.23 (m, 2H), 7.46-7.54 (m, 2H) ppm; MS (DCI/NH₃) m/z 314 (M+H)⁺.

### Example 97

### (R)-5,7-dimethoxy-N-(pyrrolidin-3-yl)-1H-indole-2-carboxamide trifluoroacetate

The title compound was prepared according to Method A using (R)-tert-butyl 3-aminopyrrolidine-1-carboxylate (Aldrich) and 5,7-dimethoxy-1H-indole-2-carboxylic acid (Enamine). ¹H NMR (300 MHz, MeOH-d₄) δ 2.07-2.30 (m, 1H), 2.35-2.54 (m, 1H), 3.34-3.46 (m, 2H), 3.49-3.66 (m, 2H), 3.80 (s, 3H), 3.94 (s, 3H), 4.47-4.66 (m, J=12.0, 7.0, 5.2 Hz, 1H); 6.42 (d, J=2.0 Hz, 1H), 6.64 (d, J=2.0 Hz, 1H), 7.02 (s, 1H) ppm; MS (DCI/NH₃) m/z 290 (M+H)⁺.

### Example 98

### (R)-6-(dimethylamino)-N-(pyrrolidin-3-yl)-1H-indole-2-carboxamide

The title compound was prepared according to Method A using (R)-tert-butyl 3-aminopyrrolidine-1-carboxylate (Aldrich) and 6-(dimethylamino)-1H-indole-2-carboxylic acid (Matrix). ¹H NMR (300 MHz, MeOH-d₄) δ 1.78-2.00 (m, 1H), 2.13-2.37 (m, 1H), 2.94 (s, 6H), 2.96-3.11 (m, 2H), 3.15-3.28 (m, 2H), 4.28-4.64 (m, 1H), 6.75 (d, J=2.4 Hz, 1H), 6.79 (dd, J=8.8, 2.4 Hz, 1H), 7.00 (s, 1H), 7.43 (d, J=8.8 Hz, 1H) ppm; MS (DCI/NH₃) m/z 273 (M+H)⁺.

### Example 99

### (R)-5,6-dimethoxy-N-(pyrrolidin-3-yl)-1H-indole-2-carboxamide

The title compound was prepared according to Method A using (R)-tert-butyl 3-aminopyrrolidine-1-carboxylate (Aldrich) and 5,6-dimethoxy-1H-indole-2-carboxylic acid (VWR). ¹H NMR (300 MHz, MeOH-d₄) δ 1.79-2.01 (m, 1H), 2.11-2.35 (m, 1H), 2.95 (dd, J=11.9, 4.7 Hz, 1H), 3.00-3.11 (m, 1H), 3.12-3.27 (m, 2H), 3.84 (s, 3H), 3.87 (s, 3H), 4.39-4.64 (m, 1H), 6.98 (s, 1H), 7.01 (d, J=0.7 Hz, 1H), 7.08 (s, 1H) ppm; MS (DCI/NH₃) m/z 290 (M+H)⁺.

### Example 100

### (R)-N-(pyrrolidin-3-yl)-1H-indole-2-carboxamide Trifluoroacetate

The title compound was prepared according to Method A using (R)-tert-butyl 3-aminopyrrolidine-1-carboxylate (Aldrich) and 1H-indole-2-carboxylic acid (Aldrich). ¹H NMR (300 MHz, MeOH-d₄) δ 2.11-2.32 (m, 1H), 2.34-2.53 (m, 1H), 3.33-3.49 (m, 2H), 3.52-3.75 (m, 2H), 4.50-4.73 (m, 1H), 7.07 (ddd, J=8.0, 7.0, 1.0 Hz, 1H), 7.11 (d, J=1.0 Hz, 1H), 7.23 (ddd, J=8.3, 7.1, 1.2 Hz, 1H), 7.44 (dd, J=8.3, 0.8 Hz, 1H), 7.56-7.64 (m, 1H) ppm; MS (DCI/NH₃) m/z 230 (M+H)⁺.

### Example 101

### (R)-5-chloro-3-methyl-N-(pyrrolidin-3-yl)-1H-indole-2-carboxamide trifluoroacetate

The title compound was prepared according to Method A using (R)-tert-butyl 3-aminopyrrolidine-1-carboxylate (Aldrich) and 5-chloro-3-methyl-1H-indole-2-carboxylic acid (ASDI). ¹H-NMR (300 MHz, MeOH-d₄) δ 2.06-2.33 (m, 1H), 2.38-2.50 (m, 1H), 2.52 (s, 3H), 3.34-3.48 (m, 2H), 3.52-3.76 (m, 2H), 4.52-4.70 (m, 1H), 7.21 (dd, J=8.8, 2.0 Hz, 1H), 7.35 (d, J=8.7 Hz, 1H), 7.60 (d, J=2.0 Hz, 1H) ppm; MS (DCI/NH₃) m/z 278, 280 (M+H)⁺.

### Example 102

### (R)-6-(dimethylamino)-N-(1-methylpyrrolidin-3-1H-indole-2-carboxamide

The title compound was prepared according to Method C using the product of Example 98. ¹H NMR (300 MHz, MeOH-d₄) δ 1.88-2.11 (m, 1H), 2.26-2.51 (m, 1H), 2.57 (s, 3H), 2.69-2.91 (m, 2H), 2.94 (s, 6H), 3.00-3.19 (m, 2H), 4.47-4.65 (m, 1H), 6.74 (d, J=2.4 Hz, 1H), 6.80 (dd, J=8.8, 2.0 Hz, 1H), 6.99 (d, J=0.7 Hz, 1H), 7.43 (d, J=8.8 Hz, 1H) ppm; MS (DCI/NH₃) m/z 287 (M+14)⁺.

### Example 103

### (R)-5,6-dimethoxy-N-(1-methylpyrrolidin-3-yl)-1H-indole-2-carboxamide

The title compound was prepared according to Method C using the product of Example 99. ¹H NMR (300 MHz, MeOH-d₄) δ 1.71-1.97 (m, 1H), 2.28-2.38 (m, 1H), 2.40 (s, 3H), 2.49-2.70 (m, 2H), 2.74-2.96 (m, 2H), 3.85 (s, 3H), 3.87 (s, 3H), 4.49-4.66 (m, 1H), 6.98 (s, 1H), 7.00 (s, 1H), 7.09 (s, 1H) ppm; MS (DCI/NH₃) m/z 304 (M+H)⁺.

### Example 104

### (R)-7-nitro-N-(piperidin-3-yl)-1H-indole-2-carboxamide

The title compound was prepared according to Method A using (R)-tert-butyl 3-aminopiperidine-1-carboxylate (Aldrich) and 7-nitro-1H-indole-2-carboxylic acid (Maybridge). ¹H NMR (300 MHz, MeOH-d₄) δ 1.59-1.64 (m, 2H), 1.74-1.92 (m, 1H), 1.99-2.17 (ml. H), 2.48-2.74 (m, 2H), 2.86-3.05 (m, 1H), 3.22 (dd, J=12.5, 3.4 Hz, 1 H), 3.94-4.23 (m, 1H), 7.30 (t, J=7.9 Hz, 1H), 7.38 (s, 1H), 8.10 (d, J=7.9 Hz, 1H), 8.27 (d, J=7.9 Hz, 1H) ppm; MS (DCI/NH₃) m/z 289 (M+H)⁺.

### Example 105

### (R)-6-(dim ethylamino)-N-(piperidin-3-yl)-1H-indole-2-carboxamide

The title compound was prepared according to Method A using (R)-tert-butyl 3-aminopiperidine-1-carboxylate (Aldrich) and 6-(dimethylamino)-1H-indole-2-carboxylic acid (Matrix). ¹H NMR (300 MHz, MeOH-d₄) δ 1.50-1.71 (m, 2H), 1.74-1.88 (m, 1H), 1.96-2.12 (m, 1H), 2.51-2.75 (m, 2H), 2.94 (s, 3H), 2.96-3.02 (m, 1H), 3.19 (dd, J=12.5, 4.4 Hz, 1H), 3.93-4.13 (m, 1H), 6.75 (d, J=2.4 Hz, 1H), 6.79 (dd, J=8.8, 2.4 Hz, 1H), 7.00 (d, J=0.7 Hz, 1H), 7.43 (d, J=8.8 Hz, 1H) ppm; MS (DCI/NH₃) m/z 287 (M+H)⁺.

### Example 106

### (R)-4,6-dimethoxy-N-(pyrrolidin-3-yl)-1H-indole-2-carboxamide

The title compound was prepared according to Method A using (R)-tert-butyl 3-aminopyrrolidine-1-carboxylate: (Aldrich) and 4,6-dimethoxy-1H-indole-2-carboxylic acid (Matrix). ¹H NMR (300 MHz, MeOH-d₄) δ 1.75-2.11 (m, 1H), 2.15-2.43 (m, 1H), 2.95 (dd, J=11.9, 4.7 Hz, 1H), 3.00-3.10 (m, 1H), 3.12-3.28 (m, 2H), 3.81 (s, 3H), 3.88 (s, 3H), 4.39-4.63 (m, 1H), 6.17 (d, J=1.7 Hz, 1H), 6.53 (d, J=1.4 Hz, 1H), 7.12 (d, J=0.7 Hz, 1H) ppm; MS (DCI/NH₃) m/z 290 (M+H)⁺.

### Example 107

### (R)-4,6-dimethoxy-N-(piperidin-3-yl)-1H-indole-2-carboxamide

The title compound was prepared according to Method A using (R)-tert-butyl 3-aminopiperidine-1-carboxylate (Aldrich) and 4,6-dimethoxy-1H-indole-2-carboxylic acid (Matrix). ¹H NMR (300 MHz, MeOH-d₄) δ 1.46-1.70 (m, 2H), 1.72-1.90 (m, 1H), 1.93-2.13 (m, 1H), 2.48-2.75 (m, 2H), 2.87-3.00 (m, 1H), 3.16 (dd, J=12.1, 3.8 Hz, 1H), 3.81 (s, 3H), 3.88 (s, 3H), 3.95-4.11 (m, 1H), 6.17 (d, J=1.6 Hz, 1H), 6.52 (d, J=1.6 Hz, 1H), 7.12 (s, 1H) ppm; MS (DCI/NH₃) m/z 304 (M+H)⁺.

### Example 108

### (R)-3,4,7-trimethyl-N-(piperidin-3-yl)-1H-indole-2-carboxamide

The title compound was prepared according to Method A using (R)-tert-butyl 3-aminopiperidine-1-carboxylate (Fluka) and 3,4,7-trimethyl-1H-indole-2-carboxylic acid (Chembridge). ¹H NMR (300 MHz, DMSO-d₆) δ 1.53-1.77 (m, 2H), 1.75-1.93 (m, 1H), 2.07 (dd, J=9.7, 3.4 Hz, 1H), 2.44 (s, 3H), 2.58-2.73 (m, 5H), 2.76 (s, 3H), 2.90-3.06 (m, 1H), 3.25 (dd, J=12.5, 4.2 Hz, 1H), 3.90-4.14 (m, 1H), 6.66 (d, J=7.1 Hz, 1H), 6.85 (d, J=7.1 Hz, 1H) ppm; MS (DCI/NH₃) m/z 286 (M+H)⁺.

### Example 109

### (R)-3,4,7-trimethyl-N-(1-methylpiperidin-3-yl)-1H-indole-2-carboxamide tosylate

The title compound was prepared according to Method C using the product of Example 108. ¹H NMR (300 MHz, DMSO-d₆) δ 1.39-1.65 (m, 1H), 1.63-1.89 (m, 1H), 1.90-2.12 (m, 2H), 2.28 (s, 3H), 2.43 (s, 3H), 2.61 (s, 3H), 2.68-2.76 (m, 4H), 2.81-2.93 (m, 4H), 3.37-3.70 (m, 2H), 4.04-4.35 (m, 1H), 6.65 (d, J=7.5 Hz, 1H), 6.86 (d, J=7.1 Hz, 1H), 7.11 (d, J=7.9 Hz, 2H), 7.48 (d, J=7.9 Hz, 2H), 8.17 (d, J=7.5 Hz, 1H), 10.64 (s, 1H) ppm; MS (DCI/NH₃) m/z 300 (M+H)⁺.

### Example 110

### (S)-3,4,7-trimethyl-N-(1-methylpyrrolidin-3-yl)-1H-indole-2-carboxamide

The title compound was prepared according to Method C using the product of Example 2. ¹H NMR (300 MHz, MeOH-d₄) δ 1.75-1.98 (m, 1H), 2.31-2.41 (m, 1H), 2.43 (s, 3H), 2.44 (s, 3H), 2.48-2.61 (m, 1H), 2.66 (s, 3H), 2.67-2.74 (m, 1H), 2.75 (s, 3H), 2.80-2.94 (m, 2H), 4.43-4.65 (m, 1H), 6.66 (dd, J=7.1, 0.7 Hz, 1H), 6.86 (d, J=7.5 Hz, 1H) ppm; MS (DCI/NH₃) m/z 286 (M+H)⁺.

### Example 111

### (R)-3,4,7-trimethyl-N-(1-methylpyrrolidin-3-yl)-1H-indole-2-carboxamide

The title compound was prepared according to Method C using the product of Example 1. ¹H NMR (300 MHz, MeOH-d₄) δ 1.76-2.03 (m, 1H), 2.30-2.41 (m, 1H), 2.42 (s, 3H), 2.44 (s, 3H), 2.47-2.59 (m, 1H), 2.66 (s, 3H), 2.66-2.72 (m, 1H), 2.75 (s, 3H), 2.79-2.93 (m, 2H), 4.43-4.65 (m, 1H), 6.66 (dd, J=7.1, 0.7 Hz, 1H), 6.85 (dd, J=7.1, 0.7 Hz, 1H) ppm; MS (DCI/NH₃) m/z 286 (M+H)⁺.

### Example 112

### (R)-N-(piperidin-3-yl)-1H-indole-2-carboxamide

The title compound was prepared according to Method A using (R)-tert-butyl 3-aminopiperidine-1-carboxylate (Fluka) and 1H-indole-2-carboxylicacid (Aldrich). ¹H NMR (300 MHz, DMSO-d₆) δ 1.48-1.71 (m, 2H), 1.71-1.87 (m, 2H), 2.72-3.01 (m, 3H), 3.09 (dd, J=11.5, 3.1 Hz, 1H), 3.96-4.92 (m, 1H), 6.90 (d, J=0.7 Hz, 1H), 7.08-7.19 (m, 1H), 7.27-7.32 (m, 1H), 7.40-7.48 (m, 1H), 7.62-7.67 (m, 1H) ppm; MS (DCI/NH₃) m/z 244 (M+H)⁺.

### Example 113

### (R)-N-(1-methylpiperidin-3-yl)-1H-indole-2-carboxamide

The title compound was prepared according to Method C using the product of Example 112. ¹H NMR (300 MHz, CDCl₃) δ 1.54-1.70 (m, 2H), 1.70-1.88 (m, 2H), 2.11-2.27 (m, 1H), 2.30 (s, 3H), 2.41-2.52 (m, 1H), 2.54-2.74 (m, 2H), 4.27-4.43 (m, 1H), 6.94 (d, J=1.4 Hz, 1H), 7.13 (ddd, J=8.0, 7.0, 1.0 Hz, 1H), 7.21-7.33 (m, 1H), 7.43 (dd, J=8.3, 0.8 Hz, 1H), 7.65 (dd, J=8.1, 1.0 Hz, 1H), 9.29 (s, 1H) ppm; MS (DCI/NH₃) m/z 258 (M+H)⁺.

### Example 114

### (R)-4,6-dichloro-N-(piperidin-3-yl)-1H-indole-2-carboxamide

The title compound was prepared according to Method A using (R)-tert-butyl 3-aminopiperidine-1-carboxylate (Fluka) and 4,6-dichloro-1H-indole-2-carboxylic acid (Astatech). ¹H NMR (300 MHz, DMSO-d₆) δ 1.29-1.54 (m, 2H), 1.57-1.71 (m, 1H), 1.80-1.93 (m, 1H), 2.29-2.47 (m, 2H), 2.80 (d, J=12.3 Hz, 1H), 2.99 (dd, J=1.9, 3.2 Hz, 1 H), 3.66-3.94 (m, 1H), 7.22 (d, J=11.6 Hz, 1H), 7.32 (s, 1H), 7.41 (s, 1H), 8.37 (d, J=7.9 Hz, 1H) ppm; MS (DCI/NH₃) m/z 312 (M+H)⁺, 314 (M+H)⁺.

### Example 115

### (R)-4,6-dichloro-N-(1-methylpiperidin-3-yl)-1H-indole-2-carboxamide

The title compound was prepared according to Method C using the product of Example 114. ¹H NMR (300 MHz, DMSO-d₆) [symbol] 1.20-1.42 (m, 1H), 1.43-1.61 (m, 1H), 1.64-1.97 (m, 4H), 2.20 (s, 3H), 2.66 (d, J=11.1 Hz, 1H), 2.82 (dd, J=9.5, 2.4 Hz, 1H), 3.87-4.05 (m, 1H), 7.22 (d, J=1.6 Hz, 1H), 7.33 (s, 1H), 7.41 (s, 1H), 8.42 (d, J=7.9 Hz, 1H), 12.06 (s, 1H) ppm; MS (DCI/NH₃) m/z 326 (M+H)⁺, 328 (M+H)⁺.

### Example 116

### (R)-4,6-dichloro-N-(piperidin-3-yl)-1H-indole-2-carboxamide

The title compound was prepared according to Method A using (R)-tert-butyl 3-aminopiperidine-1-carboxylate (CNH Technology) and 7-methyl-1H-indole-2-carboxylic acid (Aldrich). ¹H NMR (300 MHz, DMSO-d₆) [symbol] 1.29-1.57 (m, 2H), 1.57-1.82 (m, 1H), 1.80-1.98 (m, 1H), 2.31-2.47 (m, 2H), 2.50 (s, 3H), 2.72-2.89 (m, 1H), 3.00 (dd, J=11.9, 3.2 Hz, 1H), 3.71-3.95 (m, 1H), 6.89-7.00 (m, 2H), 7.13 (d, J=2.0 Hz, 1H), 7.42 (dd, J=7.1, 2.0 Hz, 1H), 8.08 (d, J=7.9 Hz, 1H), 11.26 (s, 1H) ppm; MS (DCI/NH₃) m/z 258 (M+H)⁺.

### Example 117

### (R)-N-(piperidin-3-yl)-5-(trifluoromethoxy)-1H-indole-2-carboxamide

The title compound was prepared according to Method A using (R)-tert-butyl 3-aminopiperidine-1-carboxylate (CNH Technology) and 5-(trifluoromethoxy)-1H-indole-2-carboxylic acid (Maybridge). ¹H NMR (300 MHz, DMSO-d₆) [symbol] 1.28-1.57 (m, 2H), 1.59-1.73 (m, 1H), 1.80-2.01 (m, 1H), 2.35-2.48 (m, 2H), 2.82 (d, J=12.5 Hz, 1H), 3.00 (dd, J=11.5, 3.4 Hz, 1H), 3.72-3.99 (m, 1H), 7.15 (ddd, J=9.0, 2.4, 0.8 Hz, 1H), 7.23 (s, 1H), 7.49 (d, J=9.2 Hz, 1H), 7.63 (d, J=1.0 Hz, 1H), 8.25 (d, J=7.8 Hz-1H), 11.82 (s, 1H) ppm; MS (DCI/NH₃) m/z 328 (M+H)⁺.

### Example 118

### (R)-5-(benzyloxy)-N-(piperidin-3-yl)-1H-indole-2-carboxamide

The title compound was prepared according to Method A using (R)-tert-butyl 3-aminopiperidine-1-carboxylate (CNH Technology) and 5-(benzyloxy)-1H-indole-2-carboxylic acid (Maybridge). ¹H NMR (300 MHz, DMSO-d₆) [symbol] 1.23-1.52 (m, 2H), 1.57-1.70 (m, 1H), 1.76-1.97 (m, 1H), 2.30-2.48 (m, 2H), 2.79 (d, J=111.9 Hz, 1H), 2.97 (dd, J=11.7, 3.2 Hz, 1H), 3.66-3.90 (m, 1H), 5.09 (s, 2H), 6.90 (dd, J=9.0, 2.5 Hz, 1H), 7.05 (d, J=1.4 Hz, 1H), 7.16 (d, J=2.4 Hz, 1H), 7.28-7.35 (m, 2H), 7.35-7.43 (m, 2H), 7.44-7.53 (m, 2H), 8.05 (d, J=8.1 Hz, 1H), 11.39 (s, 1H) ppm; MS (DCI/NH₃) m/z 350 (M+H)⁺.

### Example 119

### (S)-4,6-dichloro-N-(piperidin-3-yl)-1H-indole-2-carboxamide

The title compound was prepared according to Method A using (S)-tert-butyl 3-aminopiperidine-1-carboxylate (CNH Technology) and 4,6-dichloro-1H-indole-2-carboxylic acid (Astatech). ¹H NMR (300 MHz, DMSO-d₆) [symbol] 1.30-1.55 (m, 2H), 1.56-1.70 (m, 1H), 1.83-2.03 (m, 1H), 2.27-2.47 (m, 2H), 2.81 (d, J=12.2 Hz, 1H), 2.99 (dd, J=11.7, 3.2 Hz, 1H), 3.72-3.90 (m, 1H), 7.23 (d, J=1.7 Hz, 1H), 7.32 (d, J=0.7 Hz, 1H), 7.41 (dd, J=1.5, 0.8 Hz, 1H), 8.40 (d, J=7.8 Hz, 1H) ppm; MS (DCI/NH₃) m/z 312 (M+H)⁺, 314 (M+H)⁺.

### Example 120

### (S)-4,6-dichloro-N-(1-methylpiperidin-3-yl)-1H-indole-2-carboxamide

The title compound was prepared according to Method C using the product of Example 119. ¹H NMR (300 MHz, DMSO-d₆) [symbol] 1.20-1.40 (m, 1H), 1.43-1.62 (m, 1H), 1.62-1.96 (m, 4H), 2.19 (s, 3H), 2.66 (d, J=10.8 Hz, 1H), 2.82 (dd, J=10.5, 2.7 Hz, 1H), 3.87-4.03 (m, 1H), 7.23 (d, J=1.7 Hz, 1H), 7.33 (d, J=1.4 Hz, 1H), 7.41 (dd, J=1.7, 1.0 Hz, 1H), 8.45 (d, J=8.1 Hz, 1H), 12.10 (s, 1H) ppm; MS (DCI/NH₃) m/z 326 (M+H)⁺, 328 (M+H)⁺.

### Example 121

### (R)-3-methyl-N-(piperidin-3-yl)-1H-indole-2-carboxamide

The title compound was prepared according to Method A using (R)-tert-butyl 3-aminopiperidine-1-carboxylate (CNH Technology) and 3-methyl-1H-indole-2-carboxylic acid (Matrix Scientific). ¹H NMR (300 MHz, DMSO-d₆) [symbol] 1.31-1.55 (m, 2H), 1.56-1.73 (m, 1H), 1.79-1.99 (m, 1H), 2.17-2.35 (m, 1H), 2.38-2.48 (m, 1H), 2.68-2.83 (m, 1H), 2.99 (dd, J=11.5, 3.4 Hz, 1H), 3.34 (s, 3H), 3.70-3.97 (m, 1H), 7.03 (ddd, J=7.9, 7.0, 1.0 Hz, 1H), 7.19 (ddd, J=8.2, 7.0, 1.0 Hz, 1H), 7.37 (d, J=8.1 Hz, 1H), 7.59 (t, J=8.0 Hz, 2H), 11.18 (s, 1H) ppm; MS (DCI/NH₃) m/z 258 (M+H)⁺.

### Example 122

### (R)-3,5-dimethyl-N-(piperidin-3-yl)-1H-indole-2-carboxamide

The title compound was prepared according to Method A using (R)-tert-butyl 3-aminopiperidine-1-carboxylate (CNH Technology) and 3,5-dimethyl-1H-indole-2-carboxylic acid (ChemBridge). ¹H NMR (300 MHz, DMSO-d₆) δ 1.30-1.54 (m, 2H), 1.55-1.73 (m, 1H), 1.77-1.97 (m, 1H), 2.16-2.33 (m, 1H), 2.38 (s, 3H), 2.39-2.44 (m, 1H), 2.46 (s, 3H), 2.70-2.86 (m, 1H), 2.98 (dd, J=11.5, 3.4 Hz, 1H), 3.69-4.00 (m, 1H), 7.02 (dd, J=8.5, 1.4 Hz, 1H), 7.26 (d, J=8.5 Hz, 1H), 7.35 (s, 1H), 7.57 (d, J=7.8 Hz, 1H), 11.04 (s, 1H) ppm; MS (DCI/NH₃) m/z 272 (M+H)⁺.

### Example 123

### (S)-3-methyl-N-(piperidin-3-yl)-1H-indole-2-carboxamide

The title compound was prepared according to Method A using (S)-tert-butyl 3-aminopiperidine-1-carboxylate (CNH Technology) and 3-methyl-1H-indole-2-carboxylic acid (Matrix). ¹H NMR (300 MHz, DMSO-d₆) [symbol] 1.29-1.58 (m, 2H), 1.57-1.74 (m, 1H), 1.80-2.02 (m, 1H), 2.39-2.55 (m, 2H), 2.49 (s, 3H), 2.71-2.85 (m, 1H), 2.99 (dd, all 1.7, 3.4 Hz, 1H), 3.67-4.13 (m, 1H), 7.03 (t, J=7.3 Hz, 1H), 7.19 (td, J=7.5, 1.2 Hz, 1H), 7.37 (d, J=8.3 Hz, 1H), 7.58 (d, J=7.9 Hz, 1H), 11.16 (s, 1H) ppm; MS (DCI/NH₃) m/z 258 (M+H)⁺.

### Example 124

### (S)-3,5-dim ethyl-N-(piperidin-3-yl)-1H-indole-2-carboxamide

The title compound was prepared according to Method A using (S)-tert-butyl 3-aminopiperidine-1-carboxylate (CNH Technology) and 3,5-dimethyl-1H-indole-2-carboxylic acid (Chembridge). ¹H NMR (300 MHz, DMSO-d₆) δ 1.40-1.63 (m, 2H), 1.66-1.81 (m, 1H), 1.83-1.97 (m, 1H), 2.38 (s, 3H), 2.46 (s, 3H), 2.53-2.71 (m, 2H), 2.93 (d, J=12.2 Hz, 1H), 3.13 (dd, J=11.9, 3.4 Hz, 1H), 3.83-4.02 (m, 1H), 7.03 (dd, J=8.3, 1.2 Hz, 1H), 7.27 (d, J=8.5 Hz, 1H), 7.35 (s, 1H), 7.65 (d, J=7.5 Hz, 1H), 11.02 (s, 1H) ppm; MS (DCI/NH₃) m/z 272 (M+H)⁺.

### Example 125

### (R)-3-methyl-N-(1-methylpiperidin-3-yl)-1H-indole-2-carboxamide

The title compound was prepared according to Method C using the product of Example 121. ¹H NMR (300 MHz, DMSO-d₆) [symbol] 1.10-1.32 (m, 1H), 1.30-1.51 (m, 1H), 1.51-1.67 (m, 1H), 1.70-1.89 (m, 2H), 2.21-2.44 (m, 4H), 2.70-3.15 (m, 2H), 3.30 (s, 3H), 3.82-4.24 (m, 1H), 7.04 (td, J=7.5, 1.0 Hz, 1H), 7.21 (td, J=7.6, 1.0 Hz, 1H), 7.38 (d, J=8.1 Hz, 1H), 7.59 (d, J=8.1 Hz, 1H), 7.75 (d, J=7.5 Hz, 1H), 11.21 (s, 1H)-ppm; MS (DCI/NH₃) m/z 272 (M+H)⁺.

### Example 126

### (R)-3,5-dimethyl-N-(1-methylpiperidin-3-yl)-1H-indole-2-carboxamide

The title compound was prepared according to Method C using the product of Example 122. ¹H NMR (300 MHz, DMSO-d₆) [symbol] 1.26-1.43 (m, 1H), 1.43-1.61 (m, 1H), 1.64-1.86 (m, 2H), 1.94-2.14 (m, 2H), 2.21 (s, 3H), 2.38 (s, 3H), 2.46 (s, 3H), 2.53-2.64 (m, 1H), 2.70-2.87 (m, 1H), 3.84-4.16 (m, 1H), 7.03 (dd, J=8.5, 1.7 Hz, 1H), 7.26 (d, J=8.5 Hz, 1H), 7.35 (s, 1H), 7.61 (d, J=8.1 Hz, 1H), 11.07 (s, 1H) ppm; MS (DCI/NH₃) m/z 286 (M+H)⁺.

### Example 127

### (S)-3-methyl-N-(1-methylpiperidin-3-yl)-1H-indole-2-carboxamide

The title compound was prepared according to Method C using the product of Example 123. ¹H NMR (300 MHz, DMSO-d₆) [symbol] 1.25-1.43 (m, 1H), 1.46-1.64 (m, 1H), 1.62-1.85 (m, 2H), 1.94-2.13 (m, 2H), 2.21 (s, 3H), 2.52-2.64 (m, 1H), 2.70-2.87 (m, 1H), 3.34 (s, 3H), 3.81-4.59 (m, 1H), 7.03 (td, J=7.5, 1.0 Hz, 1H), 7.15-7.26 (m, 1H), 7.38 (d, J=8.1 Hz, 1H), 7.58 (d, J=8.1 Hz, 1H), 7.66 (d, J=7.8 Hz, 1H), 11.21 (s, 1H) ppm; MS (DCI/NH₃) m/z 272 (M+H)⁺.

### Example 128

### (S)-3,5-dimethyl-N-(1-methylpiperidin-3-yl)-1H-indole-2-carboxamide

The title compound was prepared according to Method C using the product of Example 124. ¹H NMR (300 MHz, DMSO-d₆) δ 1.23-1.43 (m, 1H), 1.45-1.58 (m, 1H), 1.61-1.85 (m, 2H), 1.93-2.11 (m, 2H), 2.19 (s, 3H), 2.38 (s, 3H), 2.46 (s, 3-H), 2.52-2.59 (m, 1H), 2.68-2.84 (m, 1H), 3.81-4.21 (m, J=3.7 Hz, 1H), 7.02 (dd, J=8.3, 1.5 Hz, 1 H), 7.26 (d, J=8.5 Hz, 1H), 7.35 (s, 1H), 7.60 (d, J=7.8 Hz, 1H), 11.07 (s, 1H) ppm; MS (DCI/NH₃) m/z 286 (M+H)⁺.

### Example 129

### (S)-6-tert-butyl-N-(piperidin-3-yl)-1H-indole-2-carboxamide

The title compound was prepared according to Method A using (S)-tert-butyl 3-aminopiperidine-1-carboxylate (CNH Technology) and 6-tert-butyl-1H-indole-2-carboxylic acid (Enamine). ¹H NMR (300 MHz, DMSO-d₆) δ 1.32 (s, 9-H), 1.37-1.57 (m, 2H), 1.81-1.89 (m, 1H), 1.63-1.68 (m, 1H), 2.35-2.43 (m, 2H), 2.77-2.89 (m, 1H), 3.01 (dd, J=11.9, 3.4 Hz, 1H), 3.69-3.94 (m, 1H), 7.08 (d, J=1.4 Hz, 1H), 7.13 (dd, J=8.5, 1.7 Hz, 1 H), 7.34-7.39 (m, 1H), 7.51 (d, J=8.8 Hz, 1H), 8.05 (d, J=8.1 Hz, 1H), 1.36 (s, 1H) ppm; MS (DCI/NH₃) m/z 300 (M+H)⁺.

### Example 130

### (R)-4,6,7-trimethyl-N-(piperidin-3-yl)-1H-indole-2)-carboxamide

The title compound was prepared according to Method A using (R)-tert-butyl 3-aminopiperidine-1-carboxylate (CNH Technology) and 4,6,7-trimethyl-1H-indole-2-carboxylic acid (Chembridge). ¹H NMR (300 MHz, DMSO-d₆) δ 1.54-1.83 (m, 2H), 1.83-2.06 (m, 2H), 2.27 (s, 3H), 2.29 (s, 3H), 2.38 (s, 3H), 2.41 (s, 3H), 2.69-3.02 (m, 2H), 3.23 (d, J=11.9 Hz, 1H), 3.37 (dd, J=12.1, 3.8 Hz, 1H), 4.09-4.37 (m, 1H), 6.69 (s, 1H), 7.11 (d, J=7.9 Hz, 2H), 7.14 (d, J=2.0 Hz, 1H), 7.48 (4, J=7.9 Hz, 2H), 8.34 (d, J=7.5 Hz, 1H), 11.09 (s, 1H) ppm; MS (DCI/NH₃) m/z 286 (M+H)⁺.

### Example 131

### (R)-5-chloro-3-methyl-N-(piperidin-3-yl)-1H-indole-2-carboxamide

The title compound was prepared according to Method A using (R)-tert-butyl 3-aminopiperidine-1-carboxylate (CNH Technology) and 5-chloro-3-methyl-1H-indole-2-carboxylic acid (ASDI). ¹H NMR (300 MHz, DMSO-d₆) δ 1.29-1.58 (m, 2H), 1.57-1.72 (m, 1H), 1.79-1.94 (m, 1H), 2.08-2.33 (m, 1H), 2.38-2.46 (m, 1H), 2.46 (s, 3H), 2.67-2.86 (m, 1H), 2.98 (dd, J=11.5, 3.6 Hz, 1H), 3.66-3.95 (m, 1H), 7.18 (dd, J=8.7, 2.0 Hz, 1H), 7.39 (d, J=8.7 Hz, 1H), 7.59-7.71 (m, 2H), 11.39 (s, 1H) ppm; MS (DCI/NH₃) m/z 292 (M+H)⁺, 294 (M+H)⁺.

### Example 132

### (R)-4,6,7-trimethyl-N-(1-methylpiperidin-3-yl)-1H-indole-2-carboxamide

The title compound was prepared according to Method C using the product of Example 130. ¹H NMR (300 MHz, DMSO-d₆) δ 1.24-1.40 (m, 1H), 1.44-1.63 (m, 1H), 1.64-2.02 (m, 4H), 2.18 (s, 3H), 2.27 (s, 3H), 2.37 (s, 3H), 2.40 (s, 3H), 2.57-2.67 (m, J=10.7 Hz, 1H), 2.80 (dd, J=0.5, 3.4 Hz, 1H), 3.83-4.15 (m, 1H), 6.67 (s, 1H), 7.12 (d, J=2.4 Hz, 1H), 8.08 (d, J=7.9 Hz, 1H), 11.00 (s, 1H) ppm; MS (DCI/NH₃) m/z 300 (M+H)⁺.

### Example 133

### (R)-4,7-dimethyl-N-(piperidin-3-yl)-1H-indole-2-carboxamide

The title compound was prepared according to Method A using (R)-tert-butyl 3-aminopiperidine-1-carboxylate (CNH Technology) and 4,7-dimethyl-1H-indole-2-carboxylic acid (Enamine). ¹H NMR (300 MHz, DMSO-d₆) δ 1.32-1.58 (m, 2H), 1.57-1.71 (m, 1H), 1.80-1.96 (m, 1H), 2.22-2.41 (m, 2H), 2.44 (s, 3H), 2.46 (s, 3H), 2.75-2.87 (m, 1H), 3.00 (dd, J=11.5, 3.4 Hz, 1H), 3.70-3.92 (m, 1H), 6.73 (d, J=7.5 Hz, 1H), 6.85 (d, J=7.8 Hz, 1H), 7.18 (d, J=2.4 Hz, 1H), 8.08 (d, J=7.8 Hz, 1H), 11.22 (s, 1H) ppm; MS (DCI/NH₃) m/z 272 (M+H)⁺.

### Example 134

### (R)-7-fluoro-N-(piperidin-3-yl)-1H-indole-2-carboxamide

The title compound was prepared according to Method A using (R)-tert-butyl 3-aminopiperidine-1-carboxylate (CNH Technology) and 7-fluoro-1H-indole-2-carboxylic acid (Matrix). ¹H NMR (300 MHz, DMSO-d₆) δ 1.33-1.57 (m, 2H), 1.58-1.72 (m, 1H), 1.81-1.93 (m, 1H), 2.27-2.48 (m, 2H), 2.76-2.87 (m, 1H), 3.01 (dd, J=11.7, 3.4 Hz, 1H), 3.73-3.94 (m, 1H), 6.94-7.07 (m, 2H), 7.20 (d, J=3.6 Hz, 1H), 7.37-7.53 (m, 1H), 8.15 (d, J=7.9 Hz, 1H), 11.97 (s, 1H) ppm; MS (DCI/NH₃) m/z 262 (M+H)⁺.

### Example 135

### (R)-5-chloro-3-phenyl-N-(piperidin-3-yl)-1H-indole-2-carboxamide

The title compound was prepared according to Method A using (R)-tert-butyl 3-aminopiperidine-1-carboxylate (CNH Technology) and 5-chloro-3-phenyl-1H-indole-2-carboxylic acid (Chembridge). ¹H NMR (300 MHz, DMSO-d₆) δ 1.17-1.53 (m, 3H), 1.62-1.73 (m, 1H), 2.22-2.32 (m, 1H), 2.34-2.47 (m, 1H), 2.62-2.72 (m, 1H), 2.85 (dd, J=11.9, 3.2 Hz, 1H), 3.70-3.91 (m, 1H), 7.17 (d, J=7.9 Hz, 1H), 7.24 (dd, J=8.7, 2.0 Hz, 1 H), 7.35-7.45 (m, 2H), 7.45-7.56 (m, 4H), 11.96 (s, 1H) ppm; MS (DCI/NH₃) m/z 354 (M+H)⁺, 356 (M+H)⁺.

### Example 136

### (R)-5,7-dimethoxy-N-(piperidin-3-yl)-1H-indole-2-carboxamide

The title compound was prepared according to Method A using (R)-tert-butyl 3-aminopiperidine-1-carboxylate (CNH Technology) and 5,7-dimethoxy-1H-indole-2-carboxylic acid (Enamine). ¹H NMR (300 MHz, DMSO-d₆) δ 1.32-1.53 (m, 2H), 1.57-1.78 (m, 1H), 1.81-1.99 (m, 1H), 2.25-2.46 (m, 2H), 2.74-2.85 (m, 1H), 2.99 (dd, J=11.7, 3.2 Hz, 1H), 3.75 (s, 3H), 3.76-3.85 (m, 1H), 3.90 (s, 3H), 6.41 (d, J=2.0 Hz, 1 H), 6.64 (d, J=2.0 Hz, 1H), 6.94 (d, J=1.4 Hz, 1H), 8.00 (d, J=7.8 Hz, 1H), 11.22 (s, 1H) ppm; MS (DCI/NH₃) m/z 304 (M+H)⁺.

### Example 137

### (R)-4-(difluoromethoxy)-N-(piperidin-3-yl)-1H-indole-2-carboxamide

The title compound was prepared according to Method A using (R)-tert-butyl 3-aminopiperidine-1-carboxylate (CNH Technology) and 4-(difluoromethoxy)-1H-indole-2-carboxylic acid (Enamine). ¹H NMR (300 MHz, DMSO-d₆) δ 1.40-1.66 (m, 2H), 1.66-1.84 (m, 1H), 1.84-2.02 (m, 1H), 2.52-2.75 (m, 2H), 2.99 (d, J=11.9 Hz, 1H), 3.15 (dd, J=11.9, 3.2 Hz, 1H), 3.81-4.17 (m, 1H), 6.81 (d, J=7.5 Hz, 1H), 7.03-7.24 (m, 2H), 7.25-7.51 (m, 3H), 8.39 (d, J=7.9 Hz, 1H), 11.88 (s, 1H) ppm; MS (DCI/NH₃) m/z 310 (M+H)⁺.

### Example 138

### (R)-4-fluoro-N-(piperidin-3-yl)-1H-indole-2-carboxamide

The title compound was prepared according to Method A using (R)-tert-butyl 3-aminopiperidine-1-carboxylate (CNH Technology) and 4-fluoro-1H-indole-2-carboxylic acid (Enamine). ¹H NMR (300 MHz, DMSO-d₆) δ 1.35-1.65 (m, 2H), 1.61-11.74 (m, 1H), 1.80-1.94 (m, 1H), 2.36-2.52 (m, 2H), 2.88 (d, J=12.3 Hz, 1H), 3.06 (dd, J=12.1, 3.8 Hz, 1 H), 3.75-3.98 (m, 1H), 6.81 (dd, J=10.7, 7.9 Hz, 1H), 7.15 (td, J=8.0, 5.4 Hz, 1H), 7.22-7.33 (m, 2H), 8.30 (d, J=7.9 Hz, 1H), 11.91 (s, 1H) ppm; MS (DCI/NH₃) m/z 262 (M+H)⁺.

### Example 139

### (R)-7-methyl-3-phenyl-N-(piperidin-3-yl)-1H-indole-2-carboxamide

The title compound was prepared according to Method A using (R)-tert-butyl 3-aminopiperidine-1-carboxylate (CNH Technology) and 7-methyl-3-phenyl-1H-indole-2-carboxylic acid (Ryan Scientific). ¹H NMR (300 MHz, DMSO-d₆) δ 0.93-1.53 (m, 3H), 1.64-1.80 (m, 1H), 2.24 (dd, J=11.9, 8.5 Hz, 1H), 2.30-2.40 (m, 1H), 2.54 (s, 3H), 2.60-2.73 (m, 1H), 2.85 (dd, J=11.7, 3.2 Hz, 1H), 3.63-3.96 (m, 1H), 6.90-7.13 (m, 2H), 7.22-7.40 (m, 3H), 7.42-7.54 (m, 4H), 11.47 (s, 1H) ppm; MS (DCI/NH₃) m/z 334 (M+H)⁺.

### Example 140

### (R)-7-fluoro-N-(1-methylpiperidin-3-yl)-1H-indole-2-carboxamide

The title compound was prepared according to Method C using the product of Example 134. ¹H NMR (300 MHz, DMSO-d₆) δ 1.15-1.42 (m, 1H), 1.43-1.63 (m, 1H), 1.65-1.83 (m, 2H), 1.84-2.02 (m, 2H), 2.18 (s, 3H), 2.55-2.65 (m, 1H), 2.78 (dd, J=10.5, 3.7 Hz, 1H), 3.87-4.11 (m, 1H), 6.91-7.09 (m, 2H), 7.20 (d, J=3.4 Hz, 1H), 7.37-7.55 (m, 1H), 8.19 (d, J=7.8, Hz, 1H), 11.92 (s, 1H) ppm; MS (DCI/NH₃) m/z 276 (M+H)⁺.

### Example 141

### (R)-6-bromo-4-fluoro-N-(piperidin-3-yl)-1H-indole-2-carboxamide

The title compound was prepared according to Method A using (R)-tert-butyl 3-aminopiperidine-1-carboxylate (CNH Technology) and 6-bromo-4-fluoro-1H-indole-2-carboxylic acid (Enamine). ¹H NMR (300 MHz, DMSO-d₆) δ 1.33-1.55 (m, 2H), 1.58-1.75 (m, 1H), 1.80-1.97 (m, 1H), 2.32-2.44 (m, 2H), 2.71-2.86 (m, 1H), 2.98 (dd, J=12.0, 3.2 Hz, 1H), 3.70-3.94 (m, 1H), 7.07 (dd, J=9.8, 1.7Hz, 1H), 7.30 (d, J=0.7 Hz, 1H), 7.43 (dd, J=1.4, 0.7 Hz, 1H), 8.27 (d, J=8.1 Hz, 1H) ppm; MS (DCI/NH₃) m/z 340 (M+H)⁺.

### Example 142

### (R)-4-methyl-N-(piperidin-3-yl)-1H-indole-2-carboxamide

The title compound was prepared according to Method A using (R)-tert-butyl 3-aminopiperidine-1-carboxylate (CNH Technology) and 4-methyl-1H-indole-2-carboxylic acid (Enamine). ¹H NMR (300 MHz, DMSO-d₆) δ 1.47-1.77 (m, 2H), 1.80-2.03 (m, 2H), 2.49 (s, 3H), 2.65-2.93 (m, 2H), 3.11-3.38 (m, 2H), 3.86-4.52 (m, 1H), 6.83 (d, J=7.1 Hz, 1H), 6.99-7.15 (m, 1H), 7.17-7.36 (m, 2H), 8.39 (d, J=7.8 Hz, 1H), 11.56 (s, 1H) ppm; MS (DCI/NH₃) m/z 258 (M+H)⁺.

### Example 143

### (R)-4-chloro-N-piperidin-3-yl)-1H-indole-2-carboxamide

The title compound was prepared according to Method A using (R)-tert-butyl 3-aminopiperidine-1-carboxylate (CNH Technology) and 4-chloro-1H-indole-2-carboxylic acid (Enamine). ¹H NMR (300 MHz, DMSO-d₆) δ 1.32-1.56 (m, 2H), 1.58-1.72 (m, 1H), 1.80-1.96 (m, 1H), 2.22-2.46 (m, 2H), 2.80 (d, J=12.2 Hz, 1H), 2.99 (dd, J=11.9, 3.4 Hz, 1H), 3.71-3.97 (m, 1H), 7.08-7.24 (m, 2H), 7.28 (s, 1H), 7.39 (d, J=7.8 Hz, 1H), 8.30 (d, J=8.1 Hz, 1H), 11.91 (s, 1H) ppm; MS (DCI/NH₃) m/z 278 (M+H)⁺, 280 (M+H)⁺.

### Example 144

### (R)-6-bromo-N-(piperidin-3-yl)-1H-indole-2-carboxamide

The title compound was prepared according to Method A using (R)-tert-butyl 3-aminopiperidine-1-carboxylate (CNH Technology) and 6-bromo-1H-indole-2-carboxylic acid (Asymchem). ¹H NMR (300 MHz, DMSO-d₆) δ 1.26-1.56 (m, 2H), 1.59-1.73 (m, 1H), 1.80-1.95 (m, 1H), 2.32-2.45 (m, 2H), 2.82 (d, J=12.2 Hz, 1H), 3.00 (dd, J=11.7, 3.6 Hz, 1H), 3.65-4.03 (m, 1H), 7.16 (dd, J=8.8, 1.7 Hz, 1H), 7.18 (s, 1H), 7.54-7.62 (m, 2H), 8.21 (d, J=7.8 Hz, 1H), 11.68 (s, 1H) ppm; MS (DCI/NH₃) m/z 322 (M+H)⁺.

### Example 145

### (R)-6-chloro-N-(piperidin-3-yl)-1H-indole-2-carboxamide

The title compound was prepared according to Method A using (R)-4-tert-butyl 3-aminopiperidine-1-carboxylate (CNH Technology) and 6-chloro-1H-indole-2-carboxylic acid (Tyger). ¹H NMR (300 MHz, DMSO-d₆) δ 1.31-1.55 (m, 2H), 1.56-1.72 (m, 1H), 1.77-1.93 (m, 1H), 2.26-2.46 (m, 2H), 2.80 (d, J=12.3 Hz, 1H), 2.98 (dd, J=11.9, 3.6 Hz, 1H), 3.69-4.02 (m, 1H), 7.04 (dd, J=8.3, 2.0 Hz, 1H), 7.19 (s, 1H), 7.43 (d, J=1.6 Hz, 1H), 7.63 (d, J=8.3 Hz, 1H), 8.19 (d, J=7.9 Hz, 1H), 11.68 (s, 1H) ppm; MS (DCI/NH₃) m/z 278 (M+H)⁺, 280 (M+H)⁺.

### Example 146

### (R)-6-ethyl-N-(piperidin-3-yl)-1H-indole-2-carboxamide

The title compound was prepared according to Method A using (R)-tert-butyl 3-aminopiperidine-1-carboxylate (CNH Technology) and 6-ethyl-1H-indole-2-carboxylic acid (ACBblocks). ¹H NMR (300 MHz, DMSO-d₆) δ 1.21 (t, J=7.5 Hz, 3H), 1.31-1.54 (m, 2H), 1.56-1.70 (m, 1H), 1.78-1.98 (m, 1H), 2.31-2.46 (m, 2H), 2.66 (t, J=7.5 Hz, 2H), 2.80 (d, J=12.2 Hz, 1H), 2.98 (dd, J=11.9, 3.4 Hz, 1H), 3.64-4.15 (m, 1H), 6.90 (dd, J=8.1, 1.4 Hz, 1H), 7.09 (d, J=1.0 Hz, 1H), 7.21 (s, 1H), 7.49 (d, J=8.1 Hz, 1H), 8.03 (d, J=8.1 Hz, 1H), 11.37 (s, 1H) ppm; MS (DCI/NH₃) m/z 272 (M+H)⁺.

### Example 147

### (R)-5,7-dimethyl-N-(piperidin-3-yl)-1H-indole-2-carboxamide

The title compound was prepared according to Method A using (R)-tert-butyl 3-aminopiperidine-1-carboxylate (CNH Technology) and 5,7-dimethyl-1H-indole-2-carboxylic acid (Enamine). ¹H NMR (300 MHz, DMSO-d₆) δ 1.28-1.54 (m, 2H), 1.56-1.72 (m, 1H), 1.76-1.95 (m, 1H), 2.32 (s, 3H), 2.34-2.45 (m, 2H), 2.46 (s, 3H), 2.80 (d, J=12.3 Hz, 1 H), 3.00 (dd, J=11.9, 3.2 Hz, 1H), 3.72-3.96 (m, 1H), 6.80 (s, 1H), 7.03 (d, J=2.0 Hz, 1 H), 7.18 (s, 1H), 8.03 (d, J=7.9Hz, 1H), 11.14 (s, 1H) ppm; MS (DCI/NH₃) m/z 272 (M+H)⁺.

### Example 148

### (R)-5-fluoro-7-(methylsulfonyl)-N-(piperidin-3-yl)-1H-indole-2-carboxamide

The title compound was prepared according to Method A using (R)-tert-butyl 3-aminopiperidine-1-carboxylate (CNH Technology) and 5-fluoro-7-(methylsulfonyl)-1H-indole-2-carboxylic acid (Apollo). ¹H NMR (300 MHz, DMSO-d₆) δ 1.35-1.56 (m, 2H), 1.58-1.80 (m, 1H), 1.84-1.96 (m, 1H), 2.32-2.48 (m, 2H), 2.82 (d, J=12.2 Hz, 1H), 3.01 (dd, J=11.7, 3.2 Hz, 1H), 3.43 (s, 3H), 3.73-3.92 (m, 1H), 7.36 (s, 1H), 7.61 (dd, J=8.8.

2.4 Hz, 1H), 7.93 (dd, J=9.2, 2.7 Hz, 1H), 8.55 (d, J=8.1 Hz, 1H) ppm; MS (DCI/NH₃) m/z 340 (M+H)⁺.

### Example 149

### (R)-4,7-dimethoxy-N-(piperidin-3-yl)-1H-indole-2-carboxamide

The title compound was prepared according to Method A using (R)-tert-butyl 3-aminopiperidine-1-carboxylate (CNH Technology) and 4,7-dimethoxy-1H-indole-2-carboxylic acid (Enamine). ¹H NMR (300 MHz, DMSO-d₆) δ 1.27-1.55 (m, 2H), 1.56-1.71 (m, 1H), 1.77-1.95 (m, 1H), 2.25-2.48 (m, 2H), 2.78 (d, J=11.9 Hz, 1H), 2.99 (dd, J=11.9, 3.6 Hz, 1H), 3.68-3.81 (m, 1H), 3.82 (s, 3H), 3.86 (s, 3H), 6.37 (d, J=8.3 Hz, 1 H), 6.61 (d, J=8.3 Hz, 1H), 7.08 (s, 1H), 8.06 (d, J=7.9 Hz, 1H), 11.38 (s, 1H) ppm; MS (DCI/NH₃) m/z 304 (M+H)⁺.

### Example 150

### (R)-6-(methylthio)-N-(piperidin-3-yl)-1H-indole-2-carboxamide

The title compound was prepared according to Method A using (R)-tert-butyl 3-aminopiperidine-1-carboxylate (CNH Technology) and 6-(methylthio)-1H-indole-2-carboxylic acid (Enamine). ¹H NMR (300 MHz, DMSO-d₆) δ 1.25-1.56 (m, 2H), 1.55-1.70 (m, 1H), 1.78-1.97 (m, 1H), 2.24-2.44 (m, 2H), 2.80 (d, J=12.3 Hz, 1H), 2.98 (dd, J=11.7, 3.4 Hz, 1H), 3.31 (s, 3H), 3.59-3.97 (m, 1H), 6.97 (dd, J=8.3, 1.6 Hz, 1H), 7.12 (s, 1H), 7.29 (s, 1H), 7.54 (d, J=8.3 Hz, 1H), 8.09 (d, J=7.9 Hz, 1H), 11.48 (s, 1H) ppm; MS (DCI/NH₃) m/z 290 (M+H)⁺.

### Example 151

### (S)-4,6,7-trimethyl-N-(piperidin-3-yl)-1H-indole-2-carboxamide

The title compound was prepared according to Method A using (S)-tert-butyl 3-aminopiperidine-1-carboxylate (Fluka) and 4,6,7-trimethyl-1H-indole-2-carboxylic acid (Chembridge). ¹H NMR (300 MHz, DMSO-d₆) δ 1.38-1.63 (m, 2H), 1.67-1.86 (m, 1H), 1.84-1.98 (m, 1H), 2.27 (s, 3H), 2.38 (s, 3H), 2.40 (s, 3H), 2.54-2.73 (m, 2H), 2.90-3.04 (m, 1H), 3.09-3.27 (m, 1H), 3.84-4.20 (m, 1H), 6.67 (s, 1H), 7.13 (d, J=2.0 Hz, 1H), 8.19 (d, J=7.8 Hz, 1H), 11.04 (s, 1H) ppm; MS (DCI/NH₁₃) m/z 286 (M+H)⁺.

### Example 152

### (S)-N-(piperidin-3-yl)-1H-indole-2-carboxamide

The title compound was prepared according to Method A using (S)-tert-butyl 3-aminopiperidine-1-carboxylate (Fluka) and 1H-indole-2-carboxylic acid (Aldrich). ¹H NMR (300 MHz, DMSO-d₆) δ 1.31-1.56 (m, 2H), 1.60-1.75 (m, 1H), 1.79-2.06 (m, 1H), 2.30-2.46 (m, 2H), 2.74-2.87 (m, 1H), 2.99 (dd, J=11.5, 3.4 Hz, 1H), 3.71-3.94 (m, 1H), 7.02 (ddd, J=8.0, 7.0, 1.0 Hz, 1H), 7.11-7.22 (m, 2H), 7.41 (dd, J=8.3, 0.8 Hz, 1H), 7.59 (d, J=7.8 Hz, 1H), 8.09 (d, J=8.1 Hz, 1H), 11.52 (s, 1H) ppm; MS (DCI/NH₃) m/z 244 (M+H)⁺.

### Example 153

### (S)-7-methyl-N-(piperidin-3-yl)-1H-indole-2-carboxamide

The title compound was prepared according to Method A using (S)-tert-butyl 3-aminopiperidine-1-carboxylate (Fluka) and 7-methyl-1H-indole-2-carboxylic acid (Aldrich). ¹H NMR (300 MHz, DMSO-d₆) [symbol] 1.31-1.58 (m, 2H), 1.60-1.76 (m, 1H), 1.83-2.10 (m, 1H), 2.31-2.45 (m, 2H), 2.51 (s, 3H), 2.73-2.87 (m, 1H), 3.00 (dd, J=11.9, 3.6 Hz, 1H), 3.60-3.98 (m, 1H), 6.88-7.00 (m, 2H), 7.13 (d, J=2.0 Hz, 1H), 7.32-7.50 (m, 1H), 8.08 (d, J=7.9 Hz, 1H), 11.26 (s, 1H) ppm; MS (DCI/NH₃) m/z 258_(M+H)⁺.

### Example 154

### 4,6-dichloro-N-(1-methylazetidin-3-yl)-1H-indole-2-carboxamide

The title compound was prepared according to Method A using 1-methylazetidin-3-amine dihydrochloride (Oakwood) and 4,6-dichloro-1H-indole-2-carboxylic acid (Astatech). ¹H NMR (300 MHz, MeOH-d₄) δ 2.41 (s, 3H), 3.20 (td, J=7.0, 1.9 Hz, 2H), 3.77 (td, J=7.0, 2.0 Hz, 2H), 4.53-4.69 (m, 1H), 7.12 (d, J=1.7 Hz, 1H), 7.25 (d, J=1.0 Hz, 1H), 7.41 (dd, J=1.5, 0.8 Hz, 1H) ppm; MS (DCI/NH₃) m/z 298 (M+H)⁺, 300 (M+H)⁺.

### Example 155

### 4,6-dichloro-N-(piperidin-4-yl)-1H-indole-2-carboxamide p-toluenesulfonate

The title compound was prepared according to Method A using tert-butyl 4-aminopiperidine-1-carboxylate (Aldrich) and 4,6-dichloro-1H-indole-2-carboxylic acid (Astatech). ¹H NMR (300 MHz, MeOH-d₄) δ 1.80-1.94 (m, 2H), 2.18-2.24 (m, 2H), 3.04-3.26 (m, 2H), 3.37-3.59 (m, 2H), 4.04-4.29 (m, 1H), 7.13 (d, J=1.7 Hz, 1H), 7.22 (d, J=7.8 Hz, 2H), 7.25 (d, J=0.7 Hz, 1H), 7.42 (dd, J=1.5, 0.8 Hz, 1H), 7.71 (d, J=8.1 Hz, 2H) ppm; MS (DCI/NH₃) m/z 312 (M+H)⁺, 314 (M+H)⁺.

### Example 156

### (R)-N-(1-methylpiperidin-3-yl)-7-nitro-1H-indole-2-carboxamide

The title compound was prepared according to Method C using the product of Example 104. ¹H NMR (300 MHz, MeOH-d₄) δ 1.38-1.57 (m, 1H), 1.62-2.02 (m, 3H), 2.06-2.25 (m, 2H), 2.33 (s, 3H), 2.64-2.78 (m, 1H), 2.88-3.04 (m, 1H), 4.07-4.31 (m, 1H), 7.30 (t, J=8.0 Hz, 1H), 7.37 (s, 1H), 8.10 (dd, J=8.0, 0.8 Hz, 1H), 8.27 (dd, J=8.0, 0.8 Hz, 1H) ppm; MS (DCI/NH₃) m/z 303 (M+H)⁺.

### Example 157

### (R)-4,6-dimethoxy-N-(1-methylpiperidin-3-yl)-1H-indole-2-carboxamide

The title compound was prepared according to Method C using the product of Example 107. ¹H NMR (300 MHz, MeOH-d₄) δ 1.29-1.58 (m, 1H), 1.58-1.97 (m, 3H): 2.02-2.25 (m, 2H), 2.31 (s, 3H), 2.60-2.75 (m, 1H), 2.81-3.02 (m, 1H), 3.81 (s, 3H), 3.88 (s, 3H), 4.05-4.27 (m, 1H), 6.17 (d, J=1.6 Hz, 1H), 6.52 (d, J=0.8 Hz, 1H), 7.11 (s, 1H) ppm; MS (DCI/NH₃) m/z 318 (M+H)⁺.

### Example 158

### (R)-4,6-dimethoxy-N-(1-methylpyrrolidin-3-yl)-1H-indole-2-carboxamide

The title compound was prepared according to Method C using the product of Example 106. ¹H NMR (300 MHz, MeOH-d₄) δ 1.74-1.96 (m, 1H), 2.27-2.38 (m, 1H), 2.40 (s, 3H), 2.48-2.67 (m, 2H), 2.73-2.84 (m, 1. H), 2.88 (dd, J=10.3, 7.1 Hz, 1H), 3.81 (s, 3H), 3.88 (s, 3H), 4.50-4.66 (m, 1H), 6.17 (d, J=2.0 Hz, 1H), 6.52 (d, J=1.2 Hz, 1H), 7.11 (s, 1H) ppm; MS (DCI/NH₃) m/z 304 (M+H)⁺.

### Example 159

### (R)-6-fluoro-7-methyl-N-(1-methylpyrrolidin-3-yl)-1H-indole-2-carboxamide

The title compound was prepared according to Method C the product of Example 93. ¹H NMR (300 MHz, MeOH-d₄) δ 1.74-1.96 (m, 1H), 2.31-2.40 (m, 1H), 2.41 (s, 3H), 2.43 (d, J=1.6 Hz, 3-H), 2.52-2.68 (m, 2H), 2.76-2.85 (m, 1H), 2.89 (dd, J=9.9, 7.1 Hz, 1H), 4.47-4.69 (m, 1H), 6.84 (dd, J=10.3, 8.7 Hz, 1H), 7.10 (s, 1H), 7.41 (dd, J=8.7, 5.2 Hz, 1H) ppm; MS (DCI/NH₃) m/z 276 (M+H)⁺.

### Example 160

### (R)-6-(dimethylamino)-N-(1-methylpiperidin-3-yl)-1H-indole-2-carboxamide

The title compound was prepared according to Method C using the product of Example 105. ¹H NMR (300 MHz, MeOH-d₄) δ 1.37-1.55 (m, 1H), 1.58-1.97 (m, 3H), 2.03-2.25 (m, 2H), 2.31 (s, 3H), 2.58-2.76 (m, 1H), 2.84-2.96 (m, 1H), 2.94 (s, 6H), 3.99-4.30 (m, 1H), 6.72-6.83 (m, 2H), 6.99 (s, 1H), 7.43 (d, J=8.7 Hz, 1H) ppm; MS (DCI/NH₃) m/z 301 (M+H)⁺.

### COMPOSITIONS OF THE INVENTION

Another embodiment of the invention provides pharmaceutical compositions comprising a therapeutically effective amount of a compound of formula (I) in combination with a pharmaceutically acceptable carrier. The compositions comprise compounds of the invention formulated together with one or more non-toxic pharmaceutically acceptable carriers.

Another embodiment of the invention provides pharmaceutical compositions, comprising:

(i) a nicotinic receptor ligand,

(ii) an α4β2 PAM, and

(iii) at least one pharmaceutically acceptable carrier or excipient.

Another embodiment of the invention provides pharmaceutical compositions, comprising:

(i) a nicotinic receptor ligand,

(ii) the compound of formula (I), and

(iii) at least one pharmaceutically acceptable carrier or excipient.

The pharmaceutical compositions can be formulated for oral administration in solid or liquid form, for parenteral injection or for rectal administration.

The pharmaceutical compositions of this embodiment of the invention can be administered to humans and other mammals orally, rectally, parenterally, intracistemally, intravaginally, intraperitoneally, topically (as by powders, ointments or drops), buccally or as an oral or nasal spray.

Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like, and suitable mixtures thereof), vegetable oils (such as olive oil) and injectable organic esters such as ethyl oleate, or suitable mixtures thereof. Suitable fluidity of the composition may be maintained, for example, by the use of a coating such as lecithin, by the maintenance of the required particle size in the case of dispersions, and by the use of surfactants.

These compositions can also contain adjuvants such as preservative agents, wetting agents, emulsifying agents, and dispersing agents. Prevention of the action of microorganisms can be ensured by various antibacterial and antifungal agents, for example, parabens, chlorobutanol, phenol, sorbic acid, and the like. It also can be desirable to include isotonic agents, for example, sugars, sodium chloride and the like. Prolonged absorption of the injectable pharmaceutical form can be brought about by the use of agents delaying absorption, for example, aluminum monostearate and gelatin.

In some cases, in order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug can depend upon its rate of dissolution, which, in turn, may depend upon crystal size and crystalline form. Alternatively, a parenterally administered drug form can be administered by dissolving or suspending the drug in an oil vehicle.

Suspensions, in addition to the active compounds, can contain suspending agents, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, the compounds of the invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations also are prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions can be formulated according to the known art using suitable dispersing or wetting agents and suspending agents. The sterile injectable preparation also can be a sterile injectable solution, suspension or emulsion in a nontoxic, parenterally acceptable diluent or solvent such as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms, one or more compounds of the invention is mixed with at least one inert pharmaceutically acceptable carrier such as sodium citrate or dicalcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using lactose or milk sugar as well as high molecular weight polyethylene glycols.

The solid dosage forms of tablets, dragees, capsules, pills, and granules can be prepared with coatings and shells such as enteric coatings and other coatings well-known in the pharmaceutical formulating art. They can optionally contain opacifying agents and can also be of a composition that they release the active ingredient(s) only, or preferentially, in a certain part of the intestinal tract in a delayed manner. Examples of materials useful for delaying release of the active agent can include polymeric substances and waxes.

Compositions for rectal or vaginal administration are preferably suppositories which can be prepared by mixing the compounds of this invention with suitable non-irritating carriers such as cocoa butter, polyethylene glycol or a suppository wax which are solid at ambient temperature but liquid at body temperature and therefore melt in the rectum or vagina and release the active compound.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. A desired compound of the invention is admixed under sterile conditions with a pharmaceutically acceptable carrier and any needed preservatives or buffers as may be required. Ophthalmic formulation, eardrops, eye ointments, powders and solutions are also contemplated as being within the scope of this invention.

The ointments, pastes, creams and gels may contain, in addition to an active compound of this invention, animal and vegetable fats, oils, waxes, paraffins, starch, tragacanth, cellulose derivatives polyethylene glycols, silicones, bentonites, silicic acid, talc and zinc oxide, or mixtures thereof.

Powders and sprays can contain, in addition to the compounds of this invention, lactose, talc, silicic acid, aluminum hydroxide, calcium silicates and polyamide powder, or mixtures of these substances. Sprays can additionally contain customary propellants such as chlorofluorohydrocarbons.

Compounds of the invention also can be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of the invention, stabilizers, preservatives, and the like. The preferred lipids are the natural and synthetic phospholipids and phosphatidylcholines (lecithins) used separately or together.

Methods to form liposomes are known in the art. See, for example, Prescott, Ed., Methods in Cell Biology, Volume XIV, Academic Press, New York, N.Y., (1976), p 33 et seq.

Dosage forms for topical administration of a compound of this invention include powders, sprays, ointments and inhalants. The active compound is mixed under sterile conditions with a pharmaceutically acceptable carrier and any needed preservatives, buffers or propellants. Ophthalmic formulations, eye ointments, powders and solutions are also contemplated as being within the scope of this invention. Aqueous liquid compositions of the invention also are particularly useful.

The compounds of the invention can be used in the form of pharmaceutically acceptable salts derived from inorganic or organic acids.

### Methods of Use

The biological effects of the compounds of the invention result from positive allosteric modulation of an α4β2 subtype of nicotinic acetylcholine receptor. Representative compounds of the invention, represented by Examples 1-160, demonstrate α4β2 NNR PAM activity. As such, compounds and compositions of the invention are useful for the treatment of conditions and disorders related to cholinergic dysfunction and for conditions and disorders responsive to the action of NNR modulators. The method is useful for treating, preventing or both treating and preventing conditions and disorders related to α4β2 NNR PAM activity, particularly in mammals.

More particularly, the method is useful for conditions and disorders related to attention deficit disorder, attention deficit hyperactivity disorder (ADHD), Alzheimer's disease (AD), schizophrenia, mild cognitive impairment, age associated memory impairment (AAMI), senile dementia, AIDS dementia, Pick's disease, dementia associated with Lewy bodies, dementia associated with Down's syndrome, schizophrenia, smoking cessation, substance abuse including alcohol abuse, amyotrophic lateral sclerosis, Huntington's disease, diminished CNS function associated with traumatic brain injury, acute pain, post-surgical pain, chronic pain, inflammatory pain, and neuropathic pain. The method is useful for conditions and disorders characterized by neuropsychological and cognitive dysfunction, for example in Alzheimer's disease, bipolar disorder, schizophrenia. schizoaffective disorder, and other related disorders characterized by neuropsychological and cognitive dysfunction, in particular.

Compounds of the invention also are useful for treating, preventing or both treating and preventing pain, particularly in mammals. Administration of compounds of the invention is useful for treating nociceptive and neuropathic forms of pain, for example, chronic pain, analgesic pain, post-surgical pain, neuropathic pain, and diabetic neuropathy. Such compounds are particularly beneficial for reducing adverse ganglionic effects such as at gastrointestinal systems (e.g. emesis) and for enhancing the effects of NNR ligands in such treatment.

A further aspect of the invention relates to a method of selectively modulating NNR activity, for example α4β2 NNR PAM activity, in combination with a nicotinic agonist or partial agonist to improve the tolerability of therapy using such nicotinic agonist or partial agonist, which is further described herein below. When dosed in combination with NNR agonists, such compounds could enhance efficacy in various disease states including pain and cognitive deficits by preferentially modulating α4β2 activity, and enabling improved separation from potential adverse emesis, cardiovascular and other effects.

Actual dosage levels of active ingredients in the pharmaceutical compositions of this invention can be varied so as to obtain an amount of the active compound(s) that is effective to achieve the desired therapeutic response for a particular patient, compositions and mode of administration. The selected dosage level will depend upon the activity of the particular compound, the route of administration, the severity of the condition being treated and the condition and prior medical history of the patient being treated. However, it. is within the skill of the art to start doses of the compound at levels lower than required to achieve the desired therapeutic effect and to gradually increase the dosage until the desired effect is achieved.

When used in the above or other treatments, a therapeutically effective amount of one of the compounds of the invention can be employed in pure form or, where such forms exist, in a pharmaceutically acceptable salt. Alternatively, the compound can be administered as a pharmaceutical composition containing the compound of interest in combination with one or more pharmaceutically acceptable carriers. The phrase “therapeutically effective amount” of the compound of the invention means a sufficient amount of the compound to treat disorders, at a reasonable benefit/risk ratio applicable to any medical treatment. It will be understood, however, that the total daily usage of the compounds and compositions of the invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination. or coincidental with the specific compound employed; and like factors well-known in the medical arts. For example, it is well within the skill of the art to start doses of the compound at levels lower than required to achieve the desired therapeutic effect and to gradually increase the dosage until the desired effect is achieved.

The total daily dose of the compounds of this invention administered to a human or animal ranges from about 0.010 mg/kg body weight to about 500 mg/kg body weight. More preferable doses can be in the range of from about 0.010 mg/kg body weight to about 50 mg/kg body weight. If desired, the effective daily dose can be divided into multiple doses for purposes of administration. Consequently, single dose compositions may contain such amounts or submultiples thereof to make up the daily, dose. When co-administered with other nicotinic ligands (agonist, partial agonists). the dose ranges of the compounds of this invention may be adjusted to achieve desirable efficacy and tolerability profiles.

Use with Neuronal Nicotinic Acetylcholine Receptor Ligands

It has been found that the efficacy of nicotinic receptor ligands known in the art can be improved by combining the nicotinic receptor ligand, particularly an α4β2 receptor ligand (agonist, partial agonist), with compounds of the invention. i.e. a nicotinic acetylcholine receptor α4β2 subtype selective PAM. Such. combinations are highly efficient for improving the efficacy of α4β2 ligand for treatment of pain and other disease indications such as cognitive deficits when compared to administration of an α4β2 receptor ligand alone.

Nicotinic acetylcholine ligands modulate the function by altering the activity of the receptor. Suitable compounds also can be partial agonists that partially block. or partially activate the α4β2 receptor or agonists that activate the receptor. PAMs are compounds that potentiate receptor responses to acetylcholine without themselves. triggering receptor activation or desensitization, or either, of the receptor. Nicotinic acetylcholine receptor α4β2 receptor ligands suitable for the invention can include full agonists or partial agonists, and can exhibit varying degrees of selectivity towards the α4β2 receptor.

One manner for characterizing interactions with α4β2 receptor is by assessing Kᵢ values for the displacement of [³H]-cytisine binding. Typical ligands can have Kᵢ values ranging from 1 pM to 10 μM. The [3H]-cytisine binding assays have been well reported; however, further details for carrying out the assays can be obtained in International Publication No. WO 99/32480; U.S. Pat. Nos. 5,948,793 and 5,914,328; WO 2004/018607; U.S. Pat. No. 6,809,105; WO 00/71534; and U.S. Pat. No. 6,833,370.

Accordingly α4β2 receptor ligands suitable for the invention can be compounds of various chemical classes. Particularly, some examples of α4β2 receptor ligands suitable for the invention include, but are not limited to, heterocyclic ethers, N-substituted diazabicycles, and heterocyclic substituted amino azacycles (see International Publication No. WO 99/32480, published Jul. 1, 1999; U.S. Pat. No. 5,948,793, issued Sep. 7, 1999; U.S. Pat. No. 5,914,328, issued Jun. 22, 1999; International Publication No. WO 2004/0186107, published Sep. 23, 2004; U.S. Pat. No. 6,809,105, issued Oct. 26, 2004; International Publication No. WO 00/71534, published Nov. 30, 2000; U.S. Pat. No. 6,833,370, issued Dec. 21, 2004; all of which are hereby incorporated by reference in their entirety). Further description and methods for preparing the compounds have been reported in patents, patent publications, and international patent publications cited.

Various forms of pain, psychiatric and neurological disorders can be treated by concurrently administering to a patient (i.e. a human) in need thereof, an α4β2 PAM and an α4β2 receptor ligand. Such combination may be especially useful in expanding the dosage range for obtaining therapeutically beneficial effects.

Establishing such a proper dosing schedule will be readily apparent to one skilled in the art, such as a physician treating various pain. states.

The dosage range at which the α4β2 PAM and an α4β2 receptor ligand will be administered concurrently can vary widely. The specific dosage will be chosen by the patient's physician taking into account the particular compounds chosen, the severity of the patient's illness, any other medical conditions or diseases the patient is suffering from, other drugs the patient is taking and their potential to cause an interaction or adverse event, the patient's previous response to medication, and other factors.

The α4β2 PAM and an α4β2 receptor ligand should be administered concurrently in amounts that are effective to treat the patient's pain, cognitive disorder, or related condition. In more general terms, one would create a combination of the present invention by choosing a dosage of an α4β2 PAM and an α4β2 receptor ligand according to the spirit of the guidelines presented above.

In another embodiment of the invention, the method is carried out by administering an α402 PAM together with an α4β2 receptor ligand in any manner which provides effective levels of the compounds in the body at the same time.

In another embodiment of the invention, the method is carried out by administering an α4β2 PAM selected from Examples 1-160 described herein, together with an α4β2 receptor ligand in any manner which provides effective levels of the compounds in the body at the same time.

Various embodiments of the invention can be administered to humans and other mammals orally, rectally, parenterally, intracistemally, intravaginally, intraperitoneally, topically (as by powders, ointments or drops), bucally or as an oral or nasal spray. Various embodiments of the invention should be construed to cover any route of administration that is appropriate for the medications involved and for the patient. For example, transdermal administration may be very desirable for patients who are forgetful or petulant about taking oral medicine. Injections may be appropriate for patients refusing their medication. One of the drugs may be administered by one route, such as oral, and the others may be administered by the transdermal, percutaneous, intravenous, intramuscular, intranasal, intrarectal or intravaginal route, in particular circumstances. The route of administration may be varied in any way, limited by the physical properties of the drugs and the convenience of the patient and the caregiver.

### Combination Use in Pain Therapy

Based on the diversity of the mechanisms underlying chronic pain (e.g. nociceptive or neuropathic, degrees of pain intensity, various etiologies etc), currently available pain medications are not efficacious in all patients or in all pain conditions. Analgesics can be broadly categorized as non-opioid analgesics (acetaminophen and non-steroidal anti-inflammatory drugs (NSAIDs)), opioid analgesics (morphine) and adjuvant analgesics or co-analgesics (antiepileptic drugs and antidepressants). In a simplified classification, non-opioid analgesics are mostly used to relieve mild to moderate nociceptive pain, adjuvant analgesics (gabapentin, pregabalin) are used to relieve neuropathic pain, and opioid analgesics are used to treat severe pain of all origins, depending on the dose prescribed.

NNR ligands act at multiple locations throughout the pain pathway to relieve pain. NNRs are found on primary sensory neurons (periphery) where nociceptive information is initiated, in the cell body regions of these neurons (i.e. the dorsal root ganglion or DRG), the dorsal spinal cord where the first pain synapse is located, in the brainstem cell body regions that control descending innervation, as well as in the higher brain regions that integrate and perceive sensory information such as the thalamus and the cortex. The current theory supported by evidence from multiple sources (reviewed in Decker et al., Curr. Topics Med. Chem., 4: 369, 2004) is that anti-nociceptive effects of NNR ligands are mediated by activation of brain-stem nuclei with descending inhibitory inputs to the spinal cord. Additional pathways may also mediate analgesic effects of NNR agonists in persistent or neuropathic pain.

One other aspect of the invention is the potential to enhance efficacy of other medications used for treating pain. As noted above, examples of currently used drugs include opioids, gabapentin, pregabalin, duloxetine and others. Novel mechanisms such as cannabinoids, vanilloid receptor antagonists and sodium channel blockers are also being developed for the treatment of pain. For many of these mechanisms, it is emerging that a component of efficacy may be driven by activation of descending, inhibitory inputs. For example, opioid analgesics can block pain transmission, in part by increasing descending inhibitory pathways to modulate pain transmission at the spinal level (Pasternack, G. W., Clin. Neuropharmacol. 16:1, 1993; Lauretti, G. T., Expert Reviews in Neurotherapeutics, 6: 613-622, 2006). Since these drugs exert their effect via activating descending inhibitory inputs, and these pathways can be shared or commonly activated by α4β2 NNR ligands, it is anticipated that co-administration of compounds of the invention, as α4β2 selective PAMs, can lead to enhanced efficacy of other analgesic agents by amplifying the descending inhibitory control of spinal cord activation. Thus, combining compounds of the invention with such therapeutic agents for pain affords the opportunity to create analgesic medications with either a broader or superior spectrum of efficacy that would improve the treatment of chronic pain.

Accordingly, another embodiment of the invention is a method for use in treating or preventing pain, including neuropathic pain and cognitive disorders in a patient in need thereof. comprising:

(i) administering an amount of neuronal nicotinic receptor ligand to the patient. and

(ii) administering an amount of the compound of formula (1) to the patient, wherein the amounts of (i) and (ii) together are more effective in treating pain or cognitive disorders.

Another embodiment of the invention is a method for use in treating or preventing pain in a patient in need thereof, comprising:

(i) administering an amount of the compound of formula (1) to the patient; and

(ii) administering a pain medication comprising a compound selected from an opioid, gabapentin, pregabalin, duloxetine, a cannabinoid ligand, a vanilloid receptor antagonist, and a sodium channel blocker wherein a descending modulatory pathway that is shared or commonly activated via the α4β2 nicotinic receptor mechanism is activated.

### Determination of Biological Activity

One manner to characterize α4β2 PAM activity is by characterization in clonal cell lines (for example, human embryonic kidney 293 cells) expressing the human neuronal nicotinic acetylcholine receptor subtype α4β2, particularly by use of Fluorescent Image Plate Reader technology. Effects on calcium flux or membrane potential changes can be assessed. Such assays have been reported and further details for carrying out, the assays can be obtained in International Publication No. WO 2006/114400. Another method to identify and characterize allosteric modulator activity is by expressing the α4β2 subunits in Xenopus oocytes, and by measuring electrophysiological effects on ligand-evoked current responses as previously described in Curtis, L., et al., Molecular Pharmacology, 61: 127-135, 2002.

To determine the effectiveness of representative compounds of this invention as ligands for α4β2 PAM activity, the compounds of the invention can be evaluated according to the Calcium Flux Assay described below.

### Calcium Flux Assays Using Cells Expressing NNR Subtypes

Human embryonic kidney (HEK) 293 cells stably expressing human α4β2 or (3β4 combinations are grown to confluency in 162 cm² tissue culture flasks in DMEM media supplemented with 10% FBS and 25 μg/mL zeocin and 200 μg/mL hygromycin B. Cells expressing rat or ferret subunits may also be used. For assessing α3* or α7* selectivity, IMR-32 cells may also be used. IMR-32 neuroblastoma cells (ATCC) are grown to confluency in 162 cm² tissue culture flasks in minimum essential media supplemented with 10% FBS and 1 mM sodium pyruvate, 1% non-essential amino acids and 1% antibiotic-antimycotic. For the calcium flux assay, c cells are then dissociated using cell dissociation buffer and 100-150 μL per well of 3.5×10⁵ cells/mL cell suspension (˜50,000-100,000 cells/well) was plated into 96-well black plates (poly-D-lysine precoated) with clear bottom and maintained for 24-48 hours in a tissue culture incubator at 37° C. under an atmosphere of 5% CO₂: 95% air. Other clonal cell lines or primary cell cultures that express endogenous α4* nicotinic receptors may also be used in this assay. Calcium flux was measured using calcium-3 assay kit (Molecular Devices, Sunnyvale, Calif.) or fluo-4 (Invitrogen). A stock solution of the dye was prepared by dissolving each vial supplied by the vendor in Hank's balanced-salt solution buffer (HBSS) or 150 mM NMDG, 20 mM calcium chloride containing 10 mM HEPES. The stock-solution was diluted 1:20 using a the same buffer before use. The growth media was removed from the cells. The cells were loaded with 100 μL of the dye per well and incubated at room temperature for up to one hour for HEK 293 clonal stable cell lines or 30 minutes-45 minutes at 37° C. for IMR-32 cells. Fluorescence measurements were read simultaneously from all the wells by a Fluorometic Imaging Plate Reader (FLIPR) at an excitation wavelength of 480 nm and an emission wavelength of 520 nm. Baseline fluorescence was measured for the first 6 seconds at which 3× concentrations of modulator/test compounds were added to the cell plate at 50 μL and incubated for five minutes. The fluorescence intensity was captured every second for the first 1 minute followed by every 5 seconds for an additional 4 minutes. This procedure was followed by 50 μL of 4× concentration of agonist and readings were taken for a period of 3-5 minutes as described above.

The ability of test compounds to positively modulate the response (i.e., increase the response) induced by a submaximal concentration of agonist (EC_{20-30%}) such as nicotine is measured. Potentiation is measured based on peak fluorescence responses. by screening compounds at fixed concentrations or in a concentration-response manner to derive, EC₅₀ values. The concentration dependence of changes in fluorescence responses is fitted by nonlinear regression analysis (GraphPad Prism, San Diego, Calif.) to obtain EC₅₀ values. The degree of potentiation and EC₅₀ values of the test compounds are typically calculated. To enable rank ordering of potency and efficacy, data may be normalized toga reference PAM. In general, compounds of the invention selectively potentiate α4β2 NNRs, but not others including ganglionic receptors expressed in IMR-32 cells. At α4β2 receptors, compounds of the invention typically increase fluorescence responses to submaximal nicotine (considered as 100%) to values ranging from 120 to 500%. The EC₅₀ values of active compounds were determined by concentration response analysis (EC₅₀) range from about 10 nM to about 100 μM. The data demonstrate the compounds of the invention are α4β2 PAMs that potentiate receptor responses to acetylcholine without themselves triggering receptor activation or desensitization, or either, of the receptor.

Table 1 lists the results for representative compounds of the present invention. The activity (allostertc effects—potentiation of fluorescence responses) ranges are defined as follows; “a” denotes active compounds (>100%).

TABLE 1
Examples of Selected α4β2 PAMs

| Example | |
|---|---|
| No. | Activity |
| 1 | a |
| 10 | a |
| 13 | a |
| 31 | a |
| 40 | a |
| 108 | a |
| 109 | a |
| 114 | a |

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof.

What is claimed is:

1. A compound of formula (1) 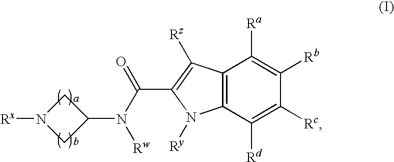
wherein
a and b are independently 1, 2, 3, or 4;
R^{a}, R^{b}, R^{c} and R^{d} are independently hydrogen, alkyl, aryl, cyano, halogen, haloalkyl, heteroaryl, NR¹R², nitro, OR³, SR¹, or SO₂R¹; or R^{a}, R^{b}, and the carbon atoms to which they rare attached taken together form a monocyclic aryl or monocyclic heteroaryl;
R¹ and R² are independently hydrogen, alkyl, arylalkyl, or cycloalkyl;
R³ is hydrogen, alkyl, arylalkyl, cycloalkyl, or haloalkyl;
R^{w} is hydrogen or alkyl;
R^{x} and R^{y} are independently hydrogen, alkyl, or cycloalkyl;
R^{z} is hydrogen, alkyl, aryl, or heteroaryl;
or a pharmaceutically acceptable salt, amide, ester or prodrug thereof.

2. The compound of claim 1, wherein b is 1.

3. The compound of claim 2, wherein a is 1.

4. The compound of claim 2, wherein a is 2.

5. The compound of claim 2, wherein a is 3.

6. The compound of claim 2, wherein a is 4.

7. The compound of claim 1, wherein a and b are each 2.

8. The compound of claim 1, wherein R^{a}, R^{b}, R^{c} and R^{d} are independently hydrogen, alkyl, halogen, or OR³, wherein R³ is alkyl or haloalkyl.

9. The compound of claim 1, wherein R^{w} is hydrogen.

10. The compound of claim 1, wherein R^{x} is hydrogen or alkyl, and R^{y} is hydrogen.

11. The compound of claim 1, wherein R^{x} is hydrogen or alkyl.

12. The compound of claim 1, selected from the group consisting of:
(R)-3,4,7-trimethyl-N-(pyrrolidin-3-yl)-1H-indole-2-carboxamide;
(S)-3,4,7-trimethyl-N-(pyrrolidin-3-yl)-1-indole-2-carboxamide;
(S)-4,6-dichloro-N-(pyrrolidin-3-yl)-1H-indole-2-carboxamide;
(R)-4,6-dimethyl-N-(pyrrolidin-3-yl)-1H-indole-2-carboxamide;
(R)-4,6-dimethyl-N-(piperidin-3-yl)-1H-indole-2-carboxamide;
(s)-3,4,7-trimethyl-N-(piperidin-3-yl)-1H-indole-2-carboxamide;
(R)-4,6-dimethyl-N-(1-methylpyrrolidin-3-yl)-1H-indole-2-carboxamide;
(R)-4,6-dimethyl-N-(1-methylpiperidin-3-yl)-1H-indole-2-carboxamide;
(S)-3,4,7-trimethyl-N-(1-methylpiperidin-3-yl)-1H-indole-2-carboxamide;
(R)-6-tert-butyl-N-(pyrrolidin-3-yl)-1H-indole-2-carboxamide;
(R)-6-tert-butyl-N-(piperidin-3-yl)-1H-indole-2-carboxamide;
(R)-6-phenyl-N-(piperidin-3-yl)-1H-indole-2-carboxamide;
(R)-6-phenyl-N-(pyrrolidin-3-yl)-1H-indole-2-carboxamide;
(R)-4,5-dimethyl-N-(pyrrolidin-3-yl)-1H-indole-2-carboxamide;
(R)-4,5-dimethyl-N-(piperidin-3-yl)-1H-indole-2-carboxamide;
N-(azepan-3-yl)-4,6-dichloro-1H-indole-2-carboxamide;
N-(azepan-3-yl)-3,4,7-trimethyl-1H-indole-2-carboxamide;
(R)-6-methoxy-N-(piperidin-3-yl)-1H-indole-2-carboxamide;
(R)-6-methoxy-N-(pyrrolidin-3-yl)-1H-indole-2-carboxamide;
(R)-6-chloro-7-methyl-N-(pyrrolidin-3-yl)-1H-indole-2-carboxamide;
(R)-6-chloro-7-methyl-N-(piperidin-3-yl)-1H-indole-2-carboxamide;
(S)-6-chloro-7-methyl-N-(pyrrolidin-3-yl)-1H-indole-2-carboxamide;
(S)-6-chloro-7-methyl-N-(piperidin-3-yl)-1H-indole-2-carboxamide;
(R)-5-bromo-N-(pyrrolidin-3-yl)-1H-indole-2-carboxamide;
(R)-1-methyl-N-(pyrrolidin-3-yl)-1H-indole-2-carboxamide;
(R)-5-bromo-N-(1-methylpyrrolidin-3-yl)-1H-indole-2-carboxamide;
(R)-4,6-dichloro-N-(pyrrolidin-3-yl)-1H-indole-2-carboxamide;
(R)-4-methoxy-N-(pyrrolidin-3-yl)-1H-indole-2-carboxamide;
(R)-5-chloro-N-(pyrrolidin-3-yl)-1H-indole-2-carboxamide;
(R)-4,6-dichloro-N-(1-methylpyrrolidin-3-yl)-1H-indole-2-carboxamide;
(R)-5-chloro-N-(1-methylpyrrolidin-3-yl)-1H-indole-2-carboxamide;
(R)-4-methoxy-N-(1-methylpyrrolidin-3-yl)-1H-indole-2-carboxamide;
(R)-N,3,4,7-tetramethyl-N-(pyrrolidin-3-yl) 1H-indole-2-carboxamide;
(R)-N,3,4,7-tetramethyl-N-(1-methylpyrrolidin-3-yl)-1H-indole-2-carboxamide;
(S)-5-fluoro-N-(pyrrolidin-3-yl)-1H-indole-2-carboxamide;
(S)-5-methoxy-N-(pyrrolidin-3-yl)-1H-indole-2-carboxamide;
(S)-5-fluoro-N-(1-methylpyrrolidin-3-yl)-1H-indole-2-carboxamide;
(S)-5-methoxy-N-(1-methylpyrrolidin-3-yl)-1H-indole-2-carboxamide;
(S)-5,6-dimethoxy-N-(pyrrolidin-3-yl)-1H-indole-2-carboxamide;
(S)-4,6-dichloro-N-(1-methylpyrrolidin-3-yl)-1H-indole-2-carboxamide;
(R)-N-(pyrrolidin-3-yl)-3H-benzo[e]indole-2-carboxamide;
(S)-N-(pyrrolidin-3-yl)-3H-benzo[e]indole-2-carboxamide;
(R)-4-(difluoromethoxy)-N-(pyrrolidin-3-yl)-1H-indole-2-carboxamide;
(S)-4-(difluoromethoxy)-N-(pyrrolidin-3-yl)-1H-indole-2-carboxamide;
(S)-7-fluoro-N-(pyrrolidin-3-yl)-1H-indole-2-carboxamide;
(S)-6-tert-butyl-N-(pyrrolidin-3-yl)-1H-indole-2-carboxamide;
(R)-4-fluoro-N-(pyrrolidin-3-yl)-1H-indole-2-carboxamide;
(S)-4-fluoro-N-(pyrrolidin-3-yl)-1H-indole-2-carboxamide;
(R)-6-(methylthio)-N-(pyrrolidin-3-yl)-1H-indole-2-carboxamide;
(S)-6-(methylthio)-N-(pyrrolidin-3-yl)-1H-indole-2-carboxamide;
(R)-3,5-dimethyl-N-(pyrrolidin-3-yl)-1H-indole-2-carboxamide;
(S)-3,5-dimethyl-1-(pyrrolidin-3-yl)-1H-indole-2-carboxamide;
(R)-4-chloro-N-(pyrrolidin-3-yl)-1H-indole-2-carboxamide;
(S)-4-chloro-N-(pyrrolidin-3-yl)-1H-indole-2-carboxamide;
(R)-4-methyl-N-(pyrrolidin-3-yl)-1H-indole-2-carboxamide;
(S)-4-methyl-N-(pyrrolidin-3-yl)-1H-indole-2-carboxamide;
(R)-3-methyl-N-(pyrrolidin-3-yl)-1H-indole-2-carboxamide;
(S)-3-methyl-N-(pyrrolidin-3-yl)-1H-indole-2-carboxamide;
(R)-N-(1-methylpyrrolidin-3-yl)-3H-benzo[e]indole-2-carboxamide;
(R)-6-chloro-N-(pyrrolidin-3-yl)-1H-indole-2-carboxamide;
(S)-6-chloro-N-(pyrrolidin-3-yl)-1H-indole-2-carboxamide;
(S)-N-(1-methylpyrrolidin-3-yl)-3H-benzo[e]indole-2-carboxamide;
(R)-6-bromo-N-(pyrrolidin-3-yl)-1H-indole-2-carboxamide;
(S)-6-bromo-N-(pyrrolidin-3-yl)-1H-indole-2-carboxamide;
(S)-7-methyl-N-(pyrrolidin-3-yl)-1H-indole-2-carboxamide;
(R)-7-methyl-N-(pyrrolidin-3-yl)-1H-indole-2-carboxamide;
(R)-4-(difluoromethoxy)-N-(1-methylpyrrolidin-3-yl)-1H-indole-2-carboxamide;
(S)-4-(difluoromethoxy)-N-(1-methylpyrrolidin-3-yl)-1H-indole-2-carboxamide;
(S)-5-chloro-N-(pyrrolidin-3-yl)-1H-indole-2-carboxamide;
(R)-3-methyl-N-(1-methylpyrrolidin-3-yl)-1H-indole-2-carboxamide;
(S)-3-methyl-N-(1-methylpyrrolidin-3-yl)-1H-indole-2-carboxamide,
(R)-4-chloro-N-(1-methylpyrrolidin-3-yl)-1H-indole-2-carboxamide;
(S)-4-chloro-N-(1-methylpyrrolidin-3-yl)-1H-indole-2-carboxamide;
(R)-6-chloro-N-(1-methylpyrrolidin-3-yl)-1H-indole-2-carboxamide;
(R)-5-methyl-N-(pyrrolidin-3-yl)-1H-indole-2-carboxamide;
(S)-5-bromo-N-(pyrrolidin-3-yl)-1H-indole-2-carboxamide;
(R)-4,6,7-trimethyl-N-(pyrrolidin-3-yl)-1H-indole-2-carboxamide;
(S)-4,6,7-trimethyl-N-(pyrrolidin-3-yl)-1H-indole-2-carboxamide;
(R)-4,7-dimethoxy-N-(pyrrolidin-3-yl)-1H-indole-2-carboxamide;
(S)-6-chloro-N-(1-methylpyrrolidin-3-yl)-1H-indole-2-carboxamide;
(S)-4,7-dimethoxy-N-(pyrrolidin-3-yl)-1H-indole-2-carboxamide;
(R)-6-bromo-N-(1-methylpyrrolidin-3-yl)-1H-indole-2-carboxamide;
(R)-7-methyl-N-(1-methylpyrrolidin-3-yl)-1H-indole-2-carboxamide;
(S)-7-methyl-N-(1-methylpyrrolidin-3-yl)-1H-indole-2-carboxamide;
(R)-5-methyl-N-(1-methylpyrrolidin-3-yl)-1H-indole-2-carboxamide;
(S)-5-bromo-N-(1-methylpyrrolidin-3-yl)-1H-indole-2-carboxamide;
(R)-4,6,7-trimethyl-N-(1-methylpyrrolidin-3-yl)-1H-indole-2-carboxamide;
(S)-4,6,7-trimethyl-N-(1-methylpyrrolidin-3-yl)-1H-indole-2-carboxamide;
(R)-4,7-dimethoxy-N-(1-methylpyrrolidin-3-yl)-1H-indole-2-carboxamide;
(S)-4,7-dimethoxy-N-(1-methylpyrrolidin-3-yl)-1H-indole-2-carboxamide.
(R)-4,7-dimethyl-N-(pyrrolidin-3-yl)-1H-indole-2-carboxamide;
(R)-6-bromo-4-fluoro-N-(pyrrolidin-3-yl)-1H-indole-2-carboxamide;
(R)-6-fluoro-7-methyl-N-(pyrrolidin-3-yl)-1H-indole-2-carboxamide;
(R)-4,7-dimethyl-N-(1-methylpyrrolidin-3-yl)-1H-indole-2-carboxamide;
(R)-6-bromo-4-fluoro-N-(1-methylpyrrolidin-3-yl)-1H-indole-2-carboxamide;
(R)-5-trifluoromethoxy-N-(pyrrolidin-3-yl)-1H-indole-2-carboxamide;
(R)-5,7-dimethoxy-N-(pyrrolidin-3-yl)-1H-indole-2-carboxamide;
(R)-6-(dimethylamino)-N-(pyrrolidin-3-yl)-1H-indole-2-carboxamide;
(R)-5,6-dimethoxy-N-(pyrrolidin-3-yl)-1H-indole-2-carboxamide;
(R)-N-(pyrrolidin-3-yl)-1H-indole-2-carboxamide;
(R)-5-chloro-3-methyl-N-(pyrrolidin-3-yl)-1H-indole-2-carboxamide;
(R)-6-(dimethylamino)-N-(1-methylpyrrolidin-3-yl)-1H-indole-2-carboxamide;
(R)-5,6-dimethoxy-N-(1-methylpyrrolidin-3-yl)-1H-indole-2-carboxamide;
(R)-7-nitro-N-(piperidin-3-yl)-1H-indole-2-carboxamide;
(R)-6-(dimethylamino)-N-(piperidin-3-yl)-1H-indole-2-carboxamide;
(R)-4,6-dimethoxy-N-(pyrrolidin-3-yl)-1H-indole-2-carboxamide;
(R)-4,6-dimethoxy-N-(piperidin-3-yl)-1H-indole-2-carboxamide;
(R)-3,4,7-trimethyl-N-(piperidin-3-yl)-1H-indole-2-carboxamide;
(R)-3,4,7-trimethyl-N-(1-methylpiperidin-3-yl)-1H-indole-2-carboxamide;
(S)-3,4,7-trimethyl-N-(1-methylpyrrolidin-3-yl)-1H-indole-2-carboxamide;
(R)-3,4,7-trimethyl-N-(1-methylpyrrolidin-3-yl)-1H-indole-2-carboxamide;
(R)-N-(piperidin-3-yl)-1H-indole-2-carboxamide;
(R)-N-(1-methylpiperidin-3-yl)-1H-indole-2-carboxamide;
(R)-4,6-dichloro-N-(piperidin-3-yl)-1H-indole-2-carboxamide;
(R)-4,6-dichloro-N-(1-methylpiperidin-3-yl)-1H-indole-2-carboxamide;
(R)-4,6-dichloro-N-(piperidin-3-yl)-1H-indole-2-carboxamide;
(R)-N-(piperidin-3-yl)-5-(trifluoromethoxy)-1H-indole-2-carboxamide;
(R)-5-(benzyloxy)-N-(piperidin-3-yl)-1H-indole-2-carboxamide;
(S)-4,6-dichloro-N-(piperidin-3-yl)-1H-indole-2-carboxamide;
(S)-4,6-dichloro-N-(1-methylpiperidin-3-yl)-1H-indole-2-carboxamide;
(R)-3-methyl-N-(piperidin-3-yl)-1H-indole-2-carboxamide;
(R)-3,5-dimethyl-N-(piperidin-3-yl)-1H-indole-2-carboxamide;
(S)-3-methyl-N-(piperidin-3-yl)-1H-indole-2-carboxamide;
(S)-3,5-dimethyl-N-(piperidin-3-yl)-1H-indole-2-carboxamide;
(R)-3-methyl-N-(1-methylpiperidin-3-yl)-1H-indole-2-carboxamide;
(R)-3,5-dimethyl-N-(1-methylpiperidin-3-yl)-1H-indole-2-carboxamide;
(S)-3-methyl-N-(1-methylpiperidin-3-yl)-1H-indole-2-carboxamide;
(S)-3,5-dimethyl-N-(1-methyl piperidin-3-yl)-1H-indole-2-carboxamide;
(S)-6-tert-butyl-N-(piperidin-3-yl)-1H-indole-2-carboxamide;
(R)-4,6,7-trimethyl-N-(piperidin-3-yl)-1H-indole-2-carboxamide;
(R)-5-chloro-3-methyl-N-(piperidin-3-yl)-1H-indole-2-carboxamide;
(R)-4,6,7-trimethyl-N-(1-methylpiperidin-3-yl)-1H-indole-2-carboxamide;
(R)-4,7-dimethyl-N-(piperidin-3-yl)-1H-indole-2-carboxamide;
(R)-7-fluoro-N-(piperidin-3-yl)-1H-indole-2-carboxamide;
(R)-5-chloro-3-phenyl-N-(piperidin-3-yl)-1H-indole-2-carboxamide;
(R)-5,7-dimethoxy-N-(piperidin-3-yl)-1H-indole-2-carboxamide;
(R)-4-(difluoromethoxy)-N-(piperidin-3-yl)-1H-indole-2-carboxamide;
(R)-4-fluoro-N-(piperidin-3-yl)-1H-indole-2-carboxamide;
(R)-7-methyl-3-phenyl-N-(piperidin-3-yl)-1H-indole-2-carboxamide;
(R)-7-fluoro-N-(1-methylpiperidin-3-yl)-1H-indole-2-carboxamide;
(R)-6-bromo-4-fluoro-N-(piperidin-3-yl)-1H-indole-2-carboxamide,
(R)-4-methyl-N-(piperidin-3-yl)-1H-indole-2-carboxamide;
(R)-4-chloro-N-(piperidin-3-yl)-1H-indole-2-carboxamide;
(R)-6-bromo-N-(piperidin-3-yl)-1H-indole-2-carboxamide;
(R)-6-chloro-N-(piperidin-3-yl)-1H-indole-2-carboxamide;
(R)-6-ethyl-N-(piperidin-3-yl)-1H-indole-2-carboxamide;
(R)-5,7-dimethyl-N-(piperidin-3-yl)-1H-indole-2-carboxamide;
(R)-5-fluoro-7-(methylsulfonyl)-N-(piperidin-3-yl)-1H-indole-2-carboxamide;
(R)-4,7-dimethoxy-N-(piperidin-3-yl)-1H-indole-2-carboxamide;
(R)-6-(methylthio)-N-(piperidin-3-yl)-1H-indole-2-carboxamide;
(S)-4,6,7-trimethyl-N-(piperidin-3-yl)-1H-indole-2-carboxamide;
(S)-N-(piperidin-3-yl)-1H-indole-2-carboxamide;
(S)-7-methyl-N-(piperidin-3-yl)-1H-indole-2-carboxamide:
4,6-dichloro-N-(1-methylazetidin-3-yl)-1H-indole-2-carboxamide;
4,6-dichloro-N-(piperidin-4-yl)-1H-indole-2-carboxamide;
(R)-N-(1-methylpiperidin-3-yl)-7-nitro-1H-indole-2-carboxamide;
(R)-4,6-dimethoxy-N-(1-methylpiperidin-3-yl)-1H-indole-2-carboxamide;
(R)-4,6-dimethoxy-N-(1-methylpyrrolidin-3-yl)-1H-indole-2-carboxamide;
(R)-6-fluoro-7-methyl-N-(1-methylpyrrolidin-3-yl)-1H-indole-2-carboxamide; and
(R)-6-(dimethylamino)-N-(1-methylpiperidin-3-yl)-1H-indole-2-carboxamide; or a pharmaceutically acceptable salt, amide, ester or prodrug thereof.

13. A pharmaceutical composition comprising a-therapeutically effective amount of the compound of claim 1, or a salt thereof, in a pharmaceutically acceptable carrier.

14. A pharmaceutical composition, comprising:
(i) a nicotinic receptor ligand. and
(ii) an α4β2 PAM consisting of the compound of claim 1 in admixture with at least one pharmaceutically acceptable recipient.

15. AT method. for treating. or preventing a condition or disorder selected from attention deficit disorder, attention deficit hyperactivity disorder (ADHD), Alzheimer's disease (AD), bipolar disorder, mild cognitive impairment, age-associated memory impairment (AAMI), senile dementia, AIDS dementia, Pick's disease, dementia associated with Lewy bodies, dementia associated with Down's syndrome, schizophrenia, schizoaffective disorder, smoking cessation, substance abuse, alcohol abuse, Huntington's disease, diminished CNS function associated with traumatic brain injury, comprising administering a therapeutically effective amount of the compound, of claim 1, or a salt thereof, or to a subject in need thereof.

16. A method for treating or preventing a condition or disorder characterized by neuropsychological and cognitive dysfunction, comprising administering a therapeutically effective amount of the compound of claim 1, or a salt thereof, to a subject in need thereof.

17. A method for treating or preventing a condition or disorder selected from acute pain, analgesic pain, post-surgical pain, chronic pain, and inflammatory pain, comprising administering a therapeutically effective amount of the compound of claim 1, or a salt thereof, to a subject in need thereof.

18. A method for use in treating or preventing pain, including neuropathic pain and cognitive disorders in a patient in need thereof, comprising:
(i) administering an amount of neuronal nicotinic receptor ligand to the patient; and
(ii) administering an amount of the compound of claim 2 to the patient;
wherein the amounts of (i) and (ii) together are more effective in treating pain or cognitive disorders.

19. A method for use in treating or preventing pain in a patient in need thereof, comprising:
(i) administering an amount of the compound of claim 2 to the patient; and
(ii) administering a pain medication comprising a compound selected from an opioid, gabapentin, pregabalin, duloxetine, a cannabinoid ligand, a vanilloid receptor antagonist, and a sodium channel blocker wherein a descending modulatory pathway that is shared or commonly activated via the α4β2 nicotinic receptor mechanism is activated.
